SEQUENCE LISTING (22 sequences)

SEQ ID NO: 1 - DNA, 20 bp, synthetic construct
  source 1..20: mol_type = other DNA; note = B_Frag_upaY_F
cgctcggacaaagaaggacc

SEQ ID NO: 2 - DNA, 21 bp, synthetic construct
  source 1..21: mol_type = other DNA; note = B_Frag_upaY_R
acttctaccctacgacgacga

SEQ ID NO: 3 - DNA, 21 bp, synthetic construct
  source 1..21: mol_type = other DNA; note = B_Frag_PSA_MR
ggtgttccaaaagacgaacgt

SEQ ID NO: 4 - DNA, 24 bp, synthetic construct
  source 1..24: mol_type = other DNA; note = B_Frag_PSA_F
tgtgtaaatgataggaggctaggg

SEQ ID NO: 5 - DNA, 20 bp, synthetic construct
  source 1..20: mol_type = other DNA; note = rpsL_F
ccgaactctgcaatgcgtaa

SEQ ID NO: 6 - DNA, 20 bp, synthetic construct
  source 1..20: mol_type = other DNA; note = rpsL_R
cgcgaaccagtacgattgag

SEQ ID NO: 7 - DNA, 22 bp, synthetic construct
  source 1..22: mol_type = other DNA; note = Type1RM_hsdS_F
gacaatcgagatgaagaacaac

SEQ ID NO: 8 - DNA, 21 bp, synthetic construct
  source 1..21: mol_type = other DNA; note = Type1RM_hsdS_R
ccataggcgtatgatttcctg

SEQ ID NO: 9 - DNA, 305 bp, Bacteroides fragilis
  source 1..305: mol_type = genomic DNA; note = Bacteroides fragilis NCTC 9343; note = PSA
tacgtaatataaagtagatggaagaaaaggggaggggtgtttcaaaaaacgttcgtcttttggaacaccttttttcgaccttttctaaaaatcatgttcttatagaaaaaacacatatataaagattctatttcatgtatataatactcttataatcagtacactaaaatagataaagaaagaaaaactcaatttatttataaaaatattttgttgttataaaaatgtgccttacctttgtgtttataaacgaacgttttttgaaacaatttcattcgttttaccactaaaaatagtttcggaaa

SEQ ID NO: 10 - DNA, 285 bp, Bacteroides fragilis
  source 1..285: mol_type = genomic DNA; note = PSH; note = Bacteroides fragilis NCTC 9343
gaacgaagatattagatacggtttatgtttcaaaaaacgttcgtcttttgaaacatcatttttgatgaaattcagaaccggataaaaagaaaaattcaaaaagaaaatctatattttttagattccaataattcattgatttacagcatcatgaagaagataaaaatagaataaatgaatttatttataaaaatattttgctatagtaaaaatactgtttacctttgttccattgaacgaacgttttttgaaacacgccttcttttctttacaattcgattttcc

SEQ ID NO: 11 - DNA, 380 bp, Bacteroides fragilis
  source 1..380: mol_type = genomic DNA; note = Bacteroides fragilis NCTC 9343; note = IR119(SusC)
aaatccctttggaatggatatataaacagattaaccaagttctcttagaacttagttattcttgaaaaatgtaaattatatacaatattaaacactttattacgctgtaattttacatttgtcaatcagatttagttgtatattttaaaataaggagttgccaatcttatttcgcggagtaaagatagatataaagtatcaaaatacaataaatatactataatatttttaaatgctaatttctacttttttttcttcgctattcaccacctctcagaatccctttaaaagtgcttgtaatgcgttttagaactgagttctaagagaactatagccattattataacctaaattaacacatattgtttaactaaaaaaga

SEQ ID NO: 12 - DNA, 517 bp, Bacteroides fragilis
  source 1..517: mol_type = genomic DNA; note = IR124(FecR); note = Bacteroides fragilis NCTC 9343
agcagcaatataccatagttctaaaggaactaatgctatatcacataaaaaataaagaagttattattaccaattaatttattagatattatactaaatatcagaagtttaatagatagaaaaataaacaacatgtatattatagataccagtcaagaaacttttctaaaaaaaagttgctcttgtcatgtggtggtagatggggctgttgtgtatttagttttagataacacaaaataaatctaaatataattattaaaatgaataaaagtaacaaagaaactaatctgatcaagaagctattggcagatcaattggacaagaaggaacgcaagaaactctattattcaaatttaatagaaaaacaaatgagaaaacaatggaaagaaaataagaatactccggtagaaaatgagattggaaataagatatgggacaagatcgagaaccaatgcataaaagttcacaaaagaatagttcctttagaacttatccaataaatatatgttgctattat

SEQ ID NO: 13 - DNA, 358 bp, Bacteroides thetaiotaomicron
  source 1..358: mol_type = genomic DNA; note = CPS1; note = Bacteroides thetaiotaomicron VPI-5482
aataagttgatagtcagtatggatctgtttctgttacttcttaggtaacggaagtaaatttgatgcaaagatagacaaaaacacaaataagaaacaaattaaagcccactttttttagttttatctaaaatatagggtaaaaacatctttagacatgaaaaatacttattttcatgccccactccttatatacctaatttccattggtatatacccttctacttagggtgtaactctcttgacaacatatttgctgctgcttttcaaccttttttcattgttcgtttatgctttatacacttccgttacctaagaagtaacggaaacatatggggtaaaaaaacatgattctaacaaa

SEQ ID NO: 14 - DNA, 337 bp, Bacteroides thetaiotaomicron
  source 1..337: mol_type = genomic DNA; note = CPS3; note = Bacteroides thetaiotaomicron VPI-5482:
tcagcactgatgctggtgtgttacttcttaggtaacggatttaaatttgacgcaaagatagacaaaaacacatgtattaaacaaataaaaacctactttttttcgatttaaccctgaatttgaaaaaaaatgtatctgaacatgaaaaaggcctattttcatgcttaggaatttatatgcccaattttcctaagaatctctactttctctaaagccaagatgctctcaaccctcattagtcggtacttttcgactttttcctcccgatttattatgaagtgtacttttccgttacctaagaagtaacggaaaaacatggggtaaaaaagtatgattt

SEQ ID NO: 15 - DNA, 361 bp, Bacteroides thetaiotaomicron
  source 1..361: mol_type = genomic DNA; note = Bacteroides thetaiotaomicron VPI-5482:; note = CPS5
ttttcttaaaatcatatttttttaccccatgtttatccgttacttcttaggtaacgaacaaacatagggaaagctaaacagggaacttttttcaacgaaacgatgaaaagatgactataaacttggaaatagagcagatgtgaaggtgtttttcaaaaattagaataaaaaaaagacataacacgaatattgcaatattcatcagctatgcctgcatttgattcattttccgaaaataatttcatttttgtttgcgtattttcataaatatgtccatctttgcgccatgaatcattccgttacctaagaagtaacgcacaccgataaacactgtatatcaagcttctaaatcagatgcact

SEQ ID NO: 16 - DNA, 345 bp, Bacteroides thetaiotaomicron
  source 1..345: mol_type = genomic DNA; note = CPS6; note = Bacteroides thetaiotaomicron VPI-5482:
atcatatttttttaccccatgtttatccgttacttcttaggtaacgaacaagcatagggcaagcagaaaagggaacttttttcaacgaaaacatcagaagaggactggtattcaggaaataaagcagaagtgaaggtgtttttcatgaattggaataaaaaaaagacataacacggatattgtcattttccgcctgttatgcctgcatttgattcattttccgaaattaatttcatttttgtttgcgtattttcataaatatgtccatctttgcgccatgaatcattccgttacctaagaagtaacgcacaccgataaacactgtatatcaagcttctaaatcag

SEQ ID NO: 17 - DNA, 277 bp, Phocaeicola sp.
  source 1..277: mol_type = genomic DNA; note = IR299(TonB); note = Phocaeicola dorei MGYG-HGUT-02478:
tctctttttcataaaaaaactttttaatttaacgttcataaatgctaagttctcttagaacttagataaaaacgttcaaaaagagttgttttgctgagtttggcaagcaaaaagtaaatcctgttagaataaaattatttattatccaacattttagctgcgatatctttggtatttaatatattttcatatctttgcagcaatatttaatataaagttctaagagaacttaggtcatgttaaacattgaactataaggtattgacaatactcaaaa

SEQ ID NO: 18 - DNA, 345 bp, Phocaeicola sp.
  source 1..345: mol_type = genomic DNA; note = Phocaeicola dorei MGYG-HGUT-02478; note = IR300(TonB)
agttttagaaaagcaaaaactcttaaagtaccaagtaccattagaacttcgtcattcactattacttattgaaaataagtgcttgaggtctagtataaagactattactaccaatttagagtgtatttttgaaaatcatatatataaaatattttccatatacccagcatcttataacaggtaattttatatatttcattaagattataataaaaataagtatttcaattgtcgtctacaacattttttatatatttgcagcgttaatctaaattttatcgaaaagcataagttctaatggaactttgcatatatctcaataatctctaattatcaagatattac

SEQ ID NO: 19 - DNA, 325 bp, Phocaeicola sp.
  source 1..325: mol_type = genomic DNA; note = Phocaeicola dorei MGYG-HGUT-02478; note = IR301(UpxY)
atgtaagaatgttcagggatgataaaatgttaagcaaaaattatgatctctttataagagatgaactatgcgacatatatgaataaaacagtctgataataaatactttgccttcttctaaaaagcagttcgctcaacaatccgttttgcggttttaaggatcggctcgtgtcccattgttatttttttactcattttgtggttattctcaatatttttcgtaattttgcatccggttattcatctcttataaagagaagtgcaacagaagcaggtgcaaacgcacctgaaaatccgccagatataatggcaaacagacgcagat

SEQ ID NO: 20 - DNA, 357 bp, Phocaeicola sp.
  source 1..357: mol_type = genomic DNA; note = IR302(AraC); note = Phocaeicola dorei MGYG-HGUT-02478
atttacttttaatcttcacttcaacatcttcacaactatacattattatatagcatgtcatcagttatacgatagatctcttattaagagatatgttacgcacacataatacactataaatcaacgaactatattaatataagaatacttcagaagactaaatcttgtattcaaaaaacgttttttttgaatcaattattaaatttcctcgctaaattgttctaattctcaaaactttgcttaaatttgcgacaaaatatatatctcttaataagagatatacaatattttaacttggtgttttttgtataaagtacttgtaataagaggattatctccaaaatcaaagaataaaat

SEQ ID NO: 21 - DNA, 946 bp, Bacteroides ovatus
  source 1..946: mol_type = genomic DNA; note = Upxy b. ovatus
tcatacttttgattaataatttgattattgcctctcacctatcggctctacatacccgaccggcatatccgcacaagcacaccccagcatattcaaccggacagcaatcttacttctaccatctactgtaacaagctctcctacaagccccgtcaaaggacctttaataacacgtactttctcaccacgtgccaaaggagcactattcatacagattgcctcttcggaatagtcgagcataaaacggaaacgggccatttgttcatcaggaatgatagtcggactgctctcaccgcgcattaccatataccgactaactgtagaaaaagaaagaacctctttacgttctttaggatcagcatgtacaaagaccatcatgggaagcagcacggattcaaccactttacggcggtcgctccattgatgtacttcttgctggaccggtacaaaattttcaatccccattttgtctaaacgttcggctactttcttctcatgatgcatacgaaccagagcaacataccagcgtttagagtgtgctacgccttcccctgtcccaattttaggcccagcacttaaagtttctttcgttagaatcatacttttttaccccttatgtgtccgttacttcttaggtaacggataaggtatttattcacaggaaaacagatgaaaacagaaaaacatggacaagaaccgcctacagcaatgtttttctaacctacgcgaagggaaaagcatctttgaaaacagaatataggaatatcgggcatgatcaaacagaatcatcatgtcctgatatatttttgttgcatatttcaacaaatcagaaaaaaaagaagattttacttgtttactatttgtatttttgcatatctttgcaccgaataattatccgttacctaagaagtaacagaaacatttcgcccttgatttctaacacttt

SEQ ID NO: 22 - DNA, 2420 bp, Bacteroides stercorirosoris
  source 1..2420: mol_type = genomic DNA; note = Bacteroides stercorirosoris helix-turn-helix transcriptional regulator or DUF6198 family protein
gtagctcgtcactcattcacttcatcgccctgtaggccgtgggcgacatccccgtatggtgcttgaaatacctgcccagatacgactggtcggggaaattgaactggttggctatttcctgtatggaaagtgctgtggagtgaagcgtcactttgagttcaagtatcacataatcatctatccactctttggccgtgcagcccaccgtctgctgaatcgtcttactcagatgccgggtagtgacacagagtttatccgcatagtaatccaccgagcgctcctccctgaacgactctatgaccaatgctataaacctgtccttcaaccgccagcgcagatggttcgacttcgtctgccggtaaagccgttccgaccggttacaaatctcatagaaaagcatccggcaacaatagataaccagttcgcgctgatacacatttccgtcatccgcagacttctgttgcagaagccacaggtaatgattgcgtatcccatcccattcttctccctgaaacgagctataattatgttcgcgcaagaaagacactttgtccgaaggcagcgaatagaaaatctcatgccggaactgactggaaaaagcgaagaccgccaattccaattcgtccgcagcaccggcataaaacacaatctgcccggggaaaacaagaatcacgctcccggcagtcagtgcatacttctgctggttgatatagagttgcacttcaccttgcaggcagaacagaaactctccgttttccatccggcagggataggaaggcaggtccgctttccgacgagtatacaacaggtattctccttgcataaagttccgtttttgaagcacaaatatactctttaggaaattgcaacacaaaaaaatcccactttttgaacaatctgcttcgaatattgaaccgtgagaaggcagatggaagccgtaactttgcagcgactaaggtaaatgataatttaccagcgtagcgctggagcaaggttctccttcggcgtgcaagtaatctttcagacacggatgacacggatgacgcggattcttttttccttcggtgtgatatgccggtatgtatagcgcagctttaaaaatccgcgtcatccgtgtcatccgcgtctaaaagattaaagtccgcgttcgtccgccttatccgcgtagtccgcgtacccattccctcgccgataaattatcatttatcgactcgtcattatgtgaactaattgtgaacatttaggtaaaaagatttataatatggcaaaaagagaacttacagcccgttatctgtttttcatcgcgggcttatttgtgaatgcattcggcattagtttgattataaaagcgaacctgggatcatcgcccatatcaagcctcccctatacattgagtttaaaattccccgtcaccctgggacaattcaccctcctgctgaacgccctgctgatagccggacaagtatgcctgctgaaaaaagacttcaagaaagaacaattcctgcaaattcccgtagccctgcttttcagcttcttcatcgactgctccatggcgttgctggcgggttacactcccgcaggatacgccgcacagatactctcattggtggcaggctgcgccatactcggtttaggagtgagcatggaagtaatagctaacgtagtgatgctatccggcgaagcttttgtgaaagccatcaccctgcgcaccggaaaagaattcggaatcaccaaaatcggtttcgacacaacgctggcactcctcgcctgtgccgcatccctgttgctggcaggaaccatagagggagtgcgtgaaggcacgataatcgccgcactgattgtagggcttttcgcccgcttcttcaaccgccgtctgggctttatcaataaaatgattacctttgcaccggataaaacacccattcagccttatgaagacaatcgcaatcaccgaaccggcggaaattgaagcaatcatccgcaaatgcccttactgcatggtgggcatcaccgaccaggagggtaacccctacgtagttcccatgaacttcgcctaccgggatggagtaatctatctgcactccggaccggagggcagcaaagtggaaatggtggaacgctatccgcaagtctgcatcaccttctgcgaagggcacgaactggtgtatatgcaccggcaggtggcatgcagctacagcatgaagtcgcgcagcgtgatgtgccggggcaaggtccgtttcatcgaagacatggatgaaaaacgaaacatactcgacatagtcatgcaacaatacgtcgagtatgaattcaaatacagcgaacctgccgtacgcaacgtgaaggtatgggaagtcaaagtcgaaaagatgacttgcaggtccttcggcttgcgcccgagcgaagtatagcctcccgcccct

(19) United States
(12) Patent Application Publication
(10) Pub. No.: US 2026/0209867 A1
(43) Pub. Date: Jul. 23, 2026

(54) REVERSIBLE BACTERIAL PHSE-VARIATIONS AND USES THEREOF IN DIAGNOSTIC AND THERAPEUTIC APPLICATIONS

(71) Applicants: TECHNION RESEARCH & DEVELOPMENT FOUNDATION LIMITED, Haifa (IL); MIGAL GALILEE RESEARCH INSTITUTE LTD., Kiryat Shmona (IL)

(72) Inventors: Naama GEVA-ZATORSKY, Haifa (IL); Shaqed CARASSO, Haifa (IL); Tal GEFEN, Haifa (IL); Roni KESHET, Haifa (IL); Haitham HAJJO, Haifa (IL); Rawan ZAATRY, Haifa (IL); Itai SHARON, Kiryat Shmona (IL)

(21) Appl. No.: 19/157,833

(22) PCT Filed: Feb. 21, 2024
(86) PCT No.: PCT/IL2024/050199

§ 371 Date: Aug. 19, 2025

Related U.S. Application Data
(60) Provisional application No. 63/486,196, filed on Feb. 21, 2023.

(51) Int. Cl. C12Q 1/689

(52) U.S. Cl. CPC C12Q 1/689; C12Q 2600/106; C12Q 2600/118; C12Q 2600/156

(57) ABSTRACT

The present disclosure relates to reversible phase variations in bacteria e.g., residing in a microbiome or any environment, and uses thereof in determining a physiological and/or environmental condition or state of a subject or a media and/or or habitat. The present disclosure thus provides methods and personalized therapeutic methods and kits.

### TECHNOLOGICAL FIELD

The present disclosure relates to the microbiome field. More specifically, the present disclosure relates to reversible phase variation in bacteria and uses thereof in methods and personalized medicine.

### BACKGROUND ART

References considered to be relevant as background to the presently disclosed subject matter are listed below:
[1] Moxon R, Bayliss C, Hood D. Bacterial contingency loci: The role of simple sequence DNA repeats in bacterial adaptation. Rev Genet. 2006; 40:307-333. doi: 10.1146/annurev.genet.40.110405.090442
[2] van der Woude M W, Bäumler A J. Phase and Antigenic Variation in Bacteria. Clin Microbiol Rev. 2004; 17 (3): 581. doi: 10.1128/CMR.17.3.581-611.2004
[3] Ikeda J S, Schmitt C K, Darnell S C, et al. Flagellar Phase Variation of Salmonella enterica Serovar Typhimurium Contributes to Virulence in the Murine Typhoid Infection Model but Does Not Influence Salmonella-Induced Enteropathogenesis. Infect Immun. 2001; 69 (5): 3021. doi: 10.1128/IAI.69.5.3021-3030.2001
[4] Mazmanian S K, Round J L, Kasper D L. A microbial symbiosis factor prevents intestinal inflammatory disease. Nature. 2008; 453 (7195): 620-625. doi: 10.1038/nature07008
[5] Krinos C M, Coyne M J, Weinacht K G, Tzianabos A O, Kasper D L, Comstock L E. Extensive surface diversity of a commensal microorganism by multiple DNA inversions. Nature. 2001; 414 (6863): 555-558. doi: 10.1038/35107092
[6] Surana N K, Kasper D L. The yin yang of bacterial polysaccharides: lessons learned from B. fragilis PSA. Immunol Rev. 2012; 245 (1): 13-26. doi: 10.1111/j.1600-065X.2011.01075.x
[7] Troy E B, Kasper D L. Beneficial effects of Bacteroides fragilis polysaccharides on the immune system. Front Biosci. 2010; 15 (1): 25. doi: 10.2741/3603
[8] Ramos G P, Papadakis K A. Mechanisms of Disease: Inflammatory Bowel Diseases. Mayo Clin Proc. 2019; 94 (1): 155-165. doi: 10.1016/J.MAYOCP.2018.09.013
[9] Guan Q. A Comprehensive Review and Update on the Pathogenesis of Inflammatory Bowel Disease. J Immunol Res. 2019; 2019. doi: 10.1155/2019/7247238
[10] Corridoni D, Arseneau K O, Cominelli F. Inflammatory bowel disease. Immunol Lett. 2014; 161 (2): 231-235. doi: 10.1016/J.IMLET.2014.04.004
[11] Ha C W Y, Martin A, Sepich-Poore G D, et al. Translocation of Viable Gut Microbiota to Mesenteric Adipose Drives Formation of Creeping Fat in Humans. Cell. 2020; 183 (3): 666-683.e17. doi: 10.1016/J.CELL.2020.09.009
[12] Spencer C N, McQuade J L, Gopalakrishnan V, et al. Dietary fiber and probiotics influence the gut microbiome and melanoma immunotherapy response. Science (1979). 2021; 374 (6575): 1632-1640. doi: 10.1126/science.aaz7015
[13] Lee K A, Thomas A M, Bolte L A, et al. Cross-cohort gut microbiome associations with immune checkpoint inhibitor response in advanced melanoma. Nat Med. 2022; 28 (3): 535-544. doi: 10.1038/s41591-022-01695-5
[14] McCulloch J A, Davar D, Rodrigues R R, et al. Intestinal microbiota signatures of clinical response and immune-related adverse events in melanoma patients treated with anti-PD-1. Nat Med. 2022; 28 (3): 545-556. doi: 10.1038/s41591-022-01698-2

Acknowledgement of the above references herein is not to be inferred as meaning that these are in any way relevant to the patentability of the presently disclosed subject matter.

### BACKGROUND

Phase variation is the process by which bacteria undergo frequent and reversible genomic alterations in specific loci of their genomes [1]. These genomic phase-variable alterations can be manifested by genomic sequences flanked by inverted repeats which induce ‘ON’\‘OFF’ switches of gene expression, or alterations in the transcribed sequence and thus the expressed protein [2]. Phase variation often influences the production of extracellular surface components presented on the bacterial outersurface and hence exposed to the host. Therefore, such components can confer different bacterial functional phenotypes affecting the host, including immune evasion [3] immune modulation [4], and more. The prevalence of inversion mediated phase-variable regions is high in host associated bacteria, mostly in bacteria from the Bacteroidetes phylum, which is a prevalent phylum in the human gut. Bacteroides fragilis, a common resident of the gut, can modulate its surface by expressing different polysaccharides (PS, denoted PSA-PSH). Seven out of eight distinct capsular polysaccharides of B. fragilis are regulated by phase variation in an ‘ON’/‘OFF’ manner [5]. Studies have shown that the B. fragilis polysaccharide A (PSA) can modulate the host immune system by different manners [4,6] for example by inducing regulatory T cells (Tregs) and secretion of the IL-10 anti-inflammatory cytokine. Moreover, PSA was shown to confer protection against experimental colitis [7], and thus is regarded as an anti-inflammatory polysaccharide. Inflammatory bowel diseases (IBD), ulcerative colitis (UC) and Crohn's disease (CD), are characterized by a compromised mucosal barrier, inappropriate immune activation and mislocalization of the gut microbiota [8-11]. The cause of IBD is still unclear, but genetic, immunological, and environmental factors contribute to the risk of the disease. Since the gut microbiota is considered a major environmental factor, IBD has emerged as one of the most studied diseases linked to it. So far, however, most studies have focused mainly on bacterial composition, often lacking mechanistic understanding on bacterial functions and potential functional alterations. Inflammation in the gut triggers unique conditions that include physical alterations such as abnormal pH concentrations, osmotic and oxidative stress, and altered immunological mechanisms as well as changes in the microbiome and metabolome profiles. Since the gut microbiota resides at the site of IBD inflammation, analysis of bacterial phase variations can provide new insights on microbiota-host interactions, with potential clinical implications.

### SUMMARY

A first aspect relates to a method for determining and/or diagnosing a physiological and/or environmental condition or state of a subject or a media and/or habitat (and/or environmental habitat). More specifically, in some embodiments, the method comprising the following steps: In one step (a), determining phase variation in at least one locus of at least one microorganism in at least one sample of the diagnosed subject or media and/or habitat, to obtain at least one phase variation value of the sample. The next step (b) involves classifying the subject and/or the media or environmental habitat as displaying the examined physiological and/or environmental condition or state, if the at least one phase variation value obtained for the sample in step (a), is positive with respect to a reference phase variation value pre-determined for the physiological and/or environmental condition or state, or with respect to a phase variation value determined for at least one control sample, thereby determining the physiological or environmental state for a subject or a media and/or environmental habitat. In some embodiments, the methods disclosed herein may further comprise the step of identifying at least one locus displaying phase variation, that characterizes at least one physiological and/or environmental condition or state of a subject or a media and/or habitat.

A further aspect of the preset disclosure relates to a prognostic method for predicting and assessing responsiveness of a subject suffering from a pathologic disorder, to at least one therapeutic agent or a treatment regimen comprising the at least one therapeutic agent, and optionally for monitoring disease progression. In more specific embodiments, the disclosed methods comprise the following steps. In one step (a), determining phase variation in at least one locus of at least one microorganism in at least one sample of the subject, to obtain at least one phase variation value of the sample for at least one of the loci in the at least one microorganism. Another step (b) involves classifying the subject as: (i) a responder subject to the at least one therapeutic agent or treatment regimen, if the at least one phase variation value obtained in step (a) for at least one sample obtained after the initiation of the treatment regimen and/or a sample of the subject contacted with the therapeutic agent, is negative with respect to a reference phase variation value (or cutoff value) pre-determined for the pathologic disorder, or with respect to a phase variation value determined for at least one control sample; or (ii) a non-responder subject to the at least one therapeutic agent or treatment regimen, if the at least one phase variation value obtained in step (a) for at least one sample obtained after the initiation of the treatment regimen and/or a sample of the subject contacted with the therapeutic agent, is positive with respect to a reference phase variation value (or cutoff value) pre-determined for the pathologic disorder, or with respect to a phase variation value determined for at least one control sample. Thereby, predicting and assessing responsiveness of the subject to the treatment regimen.

A further aspect of the present disclosure relates to a method for determining a personalized treatment regimen for a subject suffering from a pathologic disorder. In some embodiments, the personalized therapeutic methods disclosed herein may comprise the following steps. In one step (I), assessing responsiveness of a subject suffering from a pathologic disorder to at least one therapeutic agent or a treatment regimen comprising the at least one therapeutic agent. In some embodiments, such assessment step may be performed by (a), determining phase variation in at least one locus of at least one microorganism in at least one sample of the subject; and (b), classifying the subject as a responder or a non-responder, as defined herein above for the prognostic method. In step (II), after classification of the subject, a treatment regimen is selected based on the determined responsiveness.

A further aspect of the present disclosure relates to a method for modulating a physiological and/or environmental state and/or condition in a subject in need thereof, and/or in a media and/or habitat. More specifically, the method comprising the following steps. In step (I), determining a physiological and/or environmental condition or state of a subject or a media and/or habitat, by the steps of: (a) determining phase variation in at least one locus of at least one microorganism in at least one sample of the subject or media and/or habitat, to obtain at least one phase variation value of the sample; and (b), classifying the subject and/or media and/or habitat as displaying the physiological and/or environmental condition or state; as defined in connection with the aspect of diagnostic aspect, herein above. The next step (II), of the modulatory methods of the present disclosure concerns subjecting the subject and/or media and/or habitat classified as displaying the physiological and/or environmental condition or state to at least one physiological condition and/or compound that modulates the physiological condition and/or state, thereby modulating the physiological and/or environmental state and/or condition.

A further aspect of the present disclosure relates to a method for treating, preventing, inhibiting, reducing, eliminating, protecting or delaying the onset of at least one pathologic disorder in a subject. More specifically, in some embodiments the method comprising the steps of: Step (I) involves detecting a pathological condition in a subject by the steps of: Step (a) involves determining phase variation in at least one locus of at least one microorganism in at least one sample of the subject, to obtain at least one phase variation value of the sample for at least one of the loci in the at least one microorganism (e.g., bacteria). Step (b) for detecting the pathological disorder in the subject involves classifying the subject as affected by, and/or suffering from the pathologic disorder, if the at least one phase variation value obtained for the sample in step (a), is positive with respect to a reference phase variation value pre-determined for the pathologic disorder, or with respect to a phase variation value determined for at least one control sample, thereby detecting said pathological disorder in the subject.

The diagnostic step of the disclosed therapeutic method is followed by step (II) that involves administering to a subject classified as affected by, and/or suffering from the disorder a therapeutically effective amount of at least one therapeutic compound.

A further aspect of the present disclosure relates to a screening method for identifying and/or evaluating at least one therapeutic compound for the treatment of a pathologic disorder. In some embodiments, the method comprising the following steps. In step (a), determining phase variation in at least one locus of at least one microorganism in at least one sample contacted with a candidate compound, to obtain at least one phase variation value of the sample for at least one of the loci in the at least one microorganism (e.g., bacteria). In some embodiments, the sample is of a subject suffering from the pathologic disorder. In the next step (b), determining that the candidate compound is a therapeutic compound for the disorder if the at least one phase variation value obtained for the sample in step (a), is negative with respect to a reference phase variation value pre-determined for the pathologic disorder, or with respect to a phase variation value determined for at least one control sample not contacted with the candidate compound.

A further aspect of the present disclosure relates to a kit comprising: (a) at least one detecting molecule for identifying at least one phase variation in at least one locus of at least one microorganism in at least one sample of a subject or media and/or habitat, to obtain at least one phase variation value of the sample. In some embodiments, the kit optionally further comprises at least one of: (b) pre-determined calibration curve/s or predetermined reference phase variation value/s pre-determined for at least one physiological or environmental condition or state in a subject or a media and/or habitat; and/or (c), at least one control sample.

These and other aspects of the present disclosure will become apparent by the hand of the following description.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A-1E. Bacteroides species exhibit phase variation in health and disease

FIG. 1A. Selected significantly different invertible phase variable regions (Wilcoxon rank sum test, FDR p<0.05) in at least one comparison between Healthy, CD (Crohn's Disease) and UC (Ulcerative Colitis). The gray scale indicates the F orientation and the R orientation.

FIG. 1B. Prevalence of functional genes in proximity to invertible DNA regions significantly different between healthy individuals and IBD patients.

FIG. 1C. Significantly different invertible phase variable regions (Wilcoxon rank sum test, FDR p<0.05), in Bacteroides fragilis NCTC 9343. PSA: Polysaccharide A (SEQ ID NO: 9), PSH: Polysaccharide H (SEQ ID NO: 10). IR119 (SusC) (SEQ ID NO: 11) and IR124 (FecR) (SEQ ID NO: 12) are 2 additional types of capsular polysaccharides.

FIG. 1D. Significantly different invertible phase variable regions (Wilcoxon rank sum test, FDR p<0.05), in Bacteroides thetaiotaomicron VPI-5482. CPS: Capsular Polysaccharides CPS1, CPS3, CPS5, CPS6 (SEQ ID NOs: 13, 14, 15 AND 16, respectively). *p<0.05; **p<0.01.

FIG. 1E. Significantly different invertible phase variable regions (Wilcoxon rank sum test, FDR p<0.05), in Phocaeicola dorei isolate MGYG-HGUT-02478. *p<0.05; **p<0.01. Specifically, loci IR299 (TonB, SEQ ID NO: 17), IR300 (TonB, SEQ ID NO: 18), IR301 (UpxY, SEQ ID NO: 19), IR302 (AraC, SEQ ID NO: 20).

FIG. 2A-2J. B. fragilis Polysaccharide A's promoter genomic orientation is affected by shifts in the inflamed gut microbiome

FIG. 2A. Illustration of murine model of inflammation (DSS-induced colitis). DSS: Dextran sodium sulfate.

FIG. 2B. Ratio of B. fragilis PSA's promoter ‘ON’ orientation measured by qPCR in different days of the experiment. Data represent the median (line in box), IQR (box), and minimum/maximum (whiskers). (Wilcoxon rank sum test, *p<0.05; **p<0.01). White: Control group, Dotted pattern: DSS treated mice.

FIG. 2C. Calprotectin level (ng/ml) measured in different days of the experiment. Lines represent the standard deviations. Open circles black line: Control group, Squares broken line: DSS treated mice.

FIG. 2D. The body weight change of mice measured in different days of the experiment. Lines represent the standard deviations. Open circles black line: Control group, Squares broken line: DSS treated mice.

FIG. 2E. Ratio of B. fragilis PSA's promoter reverse orientation measured by the Phasefinder tool in different days of the experiment. Data represent the median (line in box), IQR (box), and minimum/maximum (whiskers). (Wilcoxon rank sum test, *p<0.05; **p<0.01). White: Control group, Dotted pattern: DSS treated mice.

FIG. 2F. Ratio of B. thetaiotaomicron CPS3's promoter reverse orientation measured by the Phasefinder tool in different days of the experiment. Data represent the median (line in box), IQR (box), and minimum/maximum (whiskers). (Wilcoxon rank sum test, *p<0.05; **p<0.01). White: Control group, Dotted pattern: DSS treated mice.

FIG. 2G. Volcano plot differential bacterial abundance between DSS treated mice in timepoint 0 and timepoint 6 detected by the DeSeq2 algorithm (Wald test, p<0.01). White dots indicate differentially abundant bacteria that were determined by adjusted P value<0.01 and log 2 fold change>1.5 and <1.5, respectively.

FIG. 2H. Alpha diversity (Shannon index) between groups and timepoints. Data represent the median (line in box), IQR (box), and minimum/maximum (whiskers). (Wilcoxon rank sum test, *p<0.05; **p<0.01). White: Control group, Dotted pattern: DSS treated mice.

FIG. 2I. PCoA on Bray-Curtis dissimilarity distances between groups and timepoints. Each point represents a single sample, colored according to group and timepoints: Open circles thin line: Control at timepoint 0, Open circles thick line: control at timepoint 6, Squares thin broken line: DSS treated at timepoint 0, Squares thick broken line: DSS treated at timepoint 6.

FIG. 2J. Ratio of B. fragilis PSA's promoter ‘ON’ orientation measured by qPCR in different days in monocolonized mice. Data represent the median (line in box), IQR (box), and minimum/maximum (whiskers). (Wilcoxon rank sum test, *p<0.05; **p<0.01). White: Control group, Dotted pattern: DSS treated mice.

FIG. 3A-3C. Patient's fecal filtrates modulate phase variation

FIG. 3A. Experimental scheme showing exposure of B. fragilis to fecal filtrates of IBD patients.

FIG. 3B. Ratio of the ‘ON’ orientation of the PSA promoter of B. fragilis, measured by qPCR, after ex-vivo exposure to fecal filtrates of CD patients before and after biological treatments. Data represent the median (line in box), IQR (box), and minimum/maximum (whiskers). (One-sided Wilcoxon rank sum test, *p<0.05). White frame: B. fragilis with no exposure, Sparsely dotted pattern: B. fragilis exposed to fecal filtrates of patients before treatment. Densely dotted pattern: B. fragilis exposed to fecal filtrates of patients after biological treatment of anti-TNF, either with Infliximab (HR) or with Humira (HuR). Dots represent individual experiments; lines connect experiments from the same patient; Shapes are determined by the patients' treatments, circle: HR, triangle: HuR.

FIG. 3C. Calprotectin levels (μg/ml) measured in patients feces. Data represent the median (line in box), IQR (box), and minimum/maximum (whiskers). (One-sided Wilcoxon rank sum test, *p<0.05). Dots represent samples; lines connect samples from the same patient; Shapes are determined by the patients' treatments, circle: HR, triangle: HuR.

FIG. 4A-4C. Phase variation loci in melanoma patients

FIG. 4A. Comparison of orientation of phase variable region in B. stercorirosoris choosing between helix-turn-helix transcriptional regulator or DUF6198 family protein (SEQ ID NO: 22), across cohorts between feces samples of responders and non-responders. Each point represents the ON/OFF ratio in a single sample, obtained from the patient before the immunotherapeutic treatment.

FIG. 4B. Comparison of orientation of phase variable region UPxY in B. ovatus (SEQ ID NO: 21, specifically, the IR in nucleotides 561-922) across cohorts between feces samples of responders and non-responders. Each point represents the ON/OFF ratio in a single sample. The samples from the MD Anderson cohort (round shapes) show significant difference between responders and non-responders.

FIG. 4C. Phase variation identification results for B. ovatus comparing responders to non-responders. Rows are different phase variation locations identified by number. The columns represent the samples (Gray indicate the bacteria this phase reign belongs to is not present in the sample and the area is not present. Black represents the area was not identified in the sequencing of the sample, but the bacteria is likely to be in the sample (other areas in the same bacteria were present). Blue and red represent the areas alignment, where red is reversed to the reference and blue is as reference. Light blue indicates the area was identified in the sample but no other areas from the same bacteria were identified.

### DETAILED DESCRIPTION OF THE INVENTION

Phase variations, prevalent in host-associated species, especially in the abundant gut Bacteroidales order, contribute to bacterial fitness in changing ecosystems, such as the human gut. Reversible DNA inversions lead to phase variable synthesis of numerous molecules (e.g. surface, regulatory, and other molecules), and as such, confer functional plasticity. The study disclosed herein, reveals genomic phase variation as a bacterial mechanism altered during gut inflammation, with potential implications on host physiology. By analyzing public databases of IBD patients (CD and UC), the inventors identify multiple phase-varied genomic regions in 28 different Bacteroides strains. Notably, the inventors find that not only intergenic regions (e.g. promoters) undergo phase variation in gut inflammatory conditions, but also intragenic regions (e.g. hydroxysteroid dehydrogenases (hsds)). Phase variation in intergenic and intragenic regions might alter gene expression (e.g. by re-orientation of genomic shufflons or by inverting promoter regions ON/OFF), while phase variation in intragenic regions can also lead to expression of different recombinant proteins. The most prevalent genes that phase-varied were polysaccharide utilization loci (PULs), specifically, susC/susD homologs, and polysaccharides (PS) promoters-both with immune-modulatory potential. Among the phase-variable PULs, SusC was prominent in most species. SusC, which is part of the starch-utilization system in Bacteroidetes, was recently shown to elicit T cell responses in IBD patients and healthy controls [5], suggesting that PULs that include SusC homologs, might interact with the immune system. Among the phase-variable PSs, the anti-inflammatory polysaccharide A (PSA) promoter of B. fragilis showed a higher percentage of reverse oriented reads in IBD patients compared to healthy controls, indicating that the ‘OFF’ orientation was more prevalent in patients, potentially limiting the protective effects of PSA. These results align with a previous study that focused on the PSA promoter of B. fragilis in IBD patients using PCR digestion on biopsies. To assess the dynamics of bacterial phase-variation under inflammation, longitudinally bacterial phase variation was analyzed in a DSS-induced colitis ‘humanized’ mouse model (i.e. GF mice colonized with a human microbiota). This analysis revealed that the bacterial genomic phase variation is a dynamic process which responds to gut inflammation in a reversible manner. The PSA promoter of B. fragilis phase-varied to its ‘OFF’ orientation upon disease induction, (in coherence with Calprotectin levels and weight loss) and reverted back to its ‘ON’ orientation upon alleviation of the inflammation. The inventors further find similarities in phase-variation of additional bacteria and genomic regions, such as the CPS3 promoter of B. thetaiotaomicron (turning ‘OFF’ under the inflammatory conditions). Intriguingly, the PSA promoter phase variation did not repeat in monocolonized mice treated with DSS, suggesting that environmental factors, existing in humanized mice and patients, are necessary to induce bacterial phase-variation. This led the inventors to hypothesize that factors in the gut ecosystem may trigger bacterial phase variation. To this end, the inventors applied fecal filtrates of inflamed IBD patients, before and after anti-TNF treatment, and demonstrated that filtrates of IBD patients can modulate the orientation ratios of the PSA promoter. Interestingly, filtrates of inflamed patients triggered an OFF-switch, while filtrates of treated patients triggered an ON-switch of the PSA promoter, in accordance with the patients' stool inflammation levels, measured by calprotectin. As the ‘ON’ orientation ratios shifted in both directions compared to the WT B. fragilis, those results exemplify that the bacterial phase variation is driven by the host inflammatory state. Another Bacteroides species, Bacteroides thetaiotaomicron, also possesses a phase variation mechanism for four out of its eight polysaccharides (CPS1-CPS8) that were recently shown to modify the bacteria susceptibility to bacteriophages. PSA is protective in mice models DSS (and other inflammations)—but the inventors show that under inflammatory conditions, B. fragilis in colonized mice might not be protective—because its OFF.->might be a novel intervention target. The inventors observed that the PSA phase-variations were coherent with the measured Calprotectin levels in the stool and with mice weight loss, suggesting that the phase variation of PSA is a dynamic and reversible process that is induced by inflammation.

The inflamed gut is characterized by unique conditions that can potentially drive phase variation. Physical alterations such as abnormal pH concentrations, osmotic and oxidative stress, and altered immunological mechanisms as well as changes in the microbiome and metabolome profiles.

Thus, a first aspect elates to a method for determining and/or diagnosing a physiological and/or environmental condition or state of a subject or a media and/or habitat (and/or environmental habitat). More specifically, in some embodiments, the method comprising the following steps: In one step (a), determining phase variation in at least one locus of at least one microorganism in at least one sample of the diagnosed subject or media and/or habitat, to obtain at least one phase variation value of the sample. In some embodiments, the method further involves determining if the at least one phase variation value obtained for the sample in step (a), is positive or negative with respect to a reference or standard phase variation value pre-determined for the physiological or environmental condition or state or with respect to a phase variation value determined for at least one control sample. Another step (b), involves classifying the subject and/or the media or environmental habitat as displaying the examined physiological and/or environmental condition or state, if the at least one phase variation value obtained for the sample in step (a), is positive with respect to a reference phase variation value (standard value or cutoff value) pre-determined for the physiological and/or environmental condition or state, or with respect to a phase variation value determined for at least one control sample, thereby determining the physiological or environmental state for a subject or a media and/or environmental habitat. The disclosed methods comprise the step of determining phase variation in a sample. Phase variation (or antigenic variation) as used herein, refers to a reversible switch between an “all-or-none” (on/off) expressing phase, resulting in variation in the level of expression of one or more proteins between individual cells of a clonal population. Antigenic variation mechanisms generate variations in the sequence of surface proteins resulting in the expression of different forms and structures of the antigenic proteins on the cell surface. Genetic modifications mechanism of phase variation includes for example DNA inversion, DNA recombination, transposition mechanism, slipped strand mispairings (SSM) and phase variation via differential methylation. As used herein, DNA inversion is carried out by enzymes that recognize inverted repeat regions and flip the DNA sequence in between them or which reside next to the switch. For example, if a promoter region lies within the sequence flanked by the inverted repeats this leads to shut down of gene expression.

The disclosed methods involve the use of a predetermined reference phase variation value, for use in the classification step (b). A reference phase variation value or a cutoff value or a standard for a specific state/disorder is determined by calculating or measuring phase variation values from two or more groups of subjects (such as healthy vs. disease, disease at different stages, different ethnic groups, different ages, genders, etc), or media, to determine at least one average value characterizing each group, and analyzing the difference between the average phase variation values obtained, and determining a phase variation range for a specific state/disorder. Alternatively, the reference phase variation value may be determined by collecting samples from two or more groups, calculating or determining the phase variation values for each group and classifying the next samples based on these values. Thus, the phase variation values from the tested sample are with respect to the collected samples which serve as reference samples. Alternatively, or in addition, the phase variation value may be compared to the phase variation value of a control sample. In certain embodiments, such control sample may be obtained from at least one of a healthy subject, a subject susceptible to develop a disorder, a subject suffering from a disorder at a specific stage, a subject suffering from a disorder at a different specific stage, a subject that responds to treatment, a non-responder subject, a subject in remission and a subject in relapse. In some embodiments, “positive” as indicated herein reflects a value that is within the range of a specific reference value or standard value, or a cutoff value predetermined for the diagnosed specific physiological and/or environmental condition or state of a subject or a media and/or habitat (determined for a control sample), and therefore characterizing the condition. Still further, “negative” as indicated herein reflects a value that does not fall within the range of a specific reference or standard value, or a cutoff value predetermined for the specific disorder and/or condition. It should be noted that the terms “positive” and “negative” as used herein, are also in some embodiments applicable for conditions in a subject that are pathological disorders, as specifically disclosed in connection with other aspects of the present disclosure. Still further, the present disclosure provides methods for determining a physiological and/or environmental condition or state of a subject or a media or environmental habitat. “Media” refers herein to either solid and/or liquid media such as earth, soil, water, waist, sewage, industrial samples, samples obtained from food and dairy processing (Yogurt), Petroleum, etc. Still further, “habitat” as used herein, refers to an assemblage of various microorganisms and organisms of any biological kingdom, together with their biotic and/or abiotic environment state. In some embodiments, the methods disclosed herein may further comprise the step of identifying at least one locus displaying phase variation, that characterizes at least one physiological and/or environmental condition or state of a subject or a media and/or habitat. In some embodiments, the loci identification is performed by: Step (i), involves analyzing at least one phase variable region in the genome of at least one microorganism in at least one sample of at least one subject or media and/or habitat displaying the at least one physiological and/or environmental condition or state, and in at one sample of at least one subject or media and/or habitat that do no display the at least one physiological and/or environmental condition or state. The next step (ii), involves mapping structural difference/s detected between the at least one phase variable region/s in the genome of at least one microorganism in at least one sample of at least one subject or media/habitat displaying the at least one physiological and/or environmental condition or state, and the genome of at least one microorganism in the at least one sample of at least one subject or media and/or habitat that do not display the at least one physiological and/or environmental condition or state. Thereby identifying at least one locus displaying phase variation that characterize at least one physiological and/or environmental condition or state of a subject or a media and/or habitat. It should be understood that the comparative analysis performed in samples that originated from subjects or media and/or habitat that display the diagnosed feature, may be performed throughout the entire genome of microorganisms in the sample, or alternatively in genomic regions that display structural features that are more susceptible, vulnerable to, and/or permitting, phase variations. Theses regions re referred to herein as phase variation region or phase variable region. Phase variation region refers herein to a part of the DNA that is prone to genetic or epigenetic modifications that results in phase variation. These regions may comprise various structural properties for example repeats, inversions, insertions, deletions, amplifications, methylation, etc. In some particular and non-limiting embodiments, the phase variable regions may comprise repeats. Thus, in some embodiments, the step of analyzing phase variable regions in the genome comprises sequencing genomic regions comprising repeats. As mentioned above, step (ii) of identifying at least one locus displaying phase variation, involves mapping structural difference/s. “Mapping structural difference(s)” refers herein to the activity or process of comparing, defining and/or detecting structural features or elements in phase variable regions that distinguish between two or more physiological or environmental conditions or states. Mapping may optionally further involve creating two or three-dimensional presentation (a list or a picture or a diagram) that represent the differences between the regions in the genome of microorganism that display phage variation. Still further, in some embodiments, structural difference/s detected between the at least one genome of at least one microorganism in at least one sample of a subject or media and/or habitat displaying the at least one physiological and/or environmental condition or state, and the genome of at least one microorganism in at least one sample of a subject or media/habitat that do no display the at least one physiological and/or environmental condition or state, may comprise difference in at least one of inversions, insertions, deletions, amplifications, methylation and any of the structural features. In some embodiments of the disclosed methods, the additional step of identifying at least one locus displaying phase variation that characterizes at least one physiological and/or environmental condition or state of a subject or a media/habitat, may be performed prior to determining a physiological and/or environmental condition or state of a subject or a media and/or habitat by the disclosed methods. Accordingly, the disclosed methods may comprise the steps of, first, in step (I), identifying at least one phase variation locus as described herein above for the disclosed methods. Briefly, in step (i), analyzing at least one phase variable region in the genome of at least one microorganism in at least one sample; and in step (ii), mapping structural difference/s detected between the at least one phase variable region/s in the genome of at least one microorganism, as discussed above. In next step (II), determining a physiological and/or environmental condition or state of a subject or a media and/or habitat by the steps (a) and (b), as discussed above. Briefly, (a), determining phase variation in at least one locus identified in step I (ii), and (b), classifying the subject and/or media and/or habitat as displaying the physiological and/or environmental condition or state, as detailed above.

In some embodiments, the physiological state and/or condition of a subject diagnosed by the methods of the present disclosure comprise pathological condition/s and/or health condition/s in a subject. In yet some further embodiments, the physiological state and/or conditions at least one of: an immunological state and/or condition, metabolic state, diet, behavioral/mental state and/or condition. More specifically, a “pathological condition”, refers to a condition, in which there is a disturbance of normal functioning, any abnormal condition of the body or mind that causes discomfort, dysfunction, or distress to the person affected or those in contact with that person. Pathologic condition as used herein is any condition or pathology caused by or associated with a pathogenic agent (including biotic or abiotic agents), a physical or metabolic chronic or acute stress, tissue and/or organ injury, disfunction or hyperfunction, and any of the conditions disclosed herein. It should be understood that in some embodiments, the disclosed methods may be applicable for a subject in any health condition. A “health condition” of a subject as used herein, refers to the state that subject is in, especially the physical state of the subject, that is not related to any pathology. Parameters such as physical and/or mental functioning, body structure and weight, personal factors (such as age, sex), medical test results, tissue and organ integrity and function, activity and environmental aspects are the composite health or physical state of a subject. This term encompasses any healthy, or non-diseased-homeostatic condition, for example, puberty, pregnancy, menstruation, menopause, aging, obesity, metabolic syndrome and the like.

In some embodiments, the physiological state and/or condition of a subject diagnosed by the methods of the present disclosure refers to diet. The term “diet” as used herein is the sum of food/nutrients consumed by the subject. It should be further appreciated that in conditions, which are not defined as disease states, the states determined by the methods of the present disclosure can reflect risk factors for further pathologic conditions. For example, body wight changes as in obesity and/or metabolic syndrome. In some embodiments, an immunological-state, as used herein, reflects the state of the immune system of a subject. The immune system acts to protect the host from pathogenic agents, biotic and non-biotic stimuli, in the environment (bacteria, viruses, fungi, parasites, toxins, and chemical entities). It serves to distinguish “nonself” from “self.”

In some embodiments the immunological state of the subject reflects an immune-related disorder. An “Immune-related disorder” or “Immune-mediated disorder”, as used herein encompasses any condition that is associated with the immune system of a subject, more specifically through uncontrolled modulation, inhibition or enhancement of the immune system, or that can be treated, prevented or ameliorated by reducing degradation of a certain component of the immune response in a subject, such as the adaptive or innate immune response. In some embodiments, the methods of the present disclosure may be applicable for any immune-related disorder. In more specific embodiments, immune-related disorders may comprise at least one of inflammatory disorder, an infectious disease, a proliferative disorder, an autoimmune disorder, an immune-deficiency condition, a neurodegenerative and/or cognitive and/or mental disorder, a metabolic disorder, and a condition involving at least one wound in at least one tissue and/or organ of the subject. In some embodiments, the immune-related disorder applicable in the methods of the present disclosure may be at least one infectious disease. An infectious disease as used herein encompasses any infectious disease caused by a pathogenic agent, specifically, a pathogen. More specifically, such infectious disease may be any pathological disorder caused by a pathogen. As used herein, the term “pathogen” refers to an infectious agent that causes a disease in a subject host. Pathogenic agents include prokaryotic microorganisms (bacteria, archea), lower eukaryotic microorganisms, complex eukaryotic organisms, viruses, fungi, mycoplasma, prions, parasites, for example, a parasitic protozoan, yeasts or a nematode, as well as toxins and venoms.

In some embodiments, the immune-related disorder applicable in the methods of the present disclosure may be an inflammatory disease. The terms “inflammatory disease” or “inflammatory-associated condition” refers to any disease or pathologically condition which can benefit from the reduction of at least one inflammatory parameter, for example, induction of an inflammatory cytokine such as IFN-γ, IL-2 and reduction in anti-inflammatory cytokines (IL-6, IL-10) levels. The condition may be caused (primarily) from inflammation, or inflammation may be one of the manifestations of the diseases caused by another physiological cause. In some embodiments, an inflammatory disease that may be applicable for the methods of the present disclosure may be inflammatory bowel disease (IBD). An autoimmune disorder is state in which the immune system gets directed against self cells or tissues. Autoimmune disorders include for example, but not limited to inflammatory bowel disease (IBD), ulcerative colitis (UC), Crohn's disease (CD), Systemic Lupus Erythematosus (SLE), Rheumatoid Arthritis (RA), fatty liver disease, Lymphocytic colitis, Ischaemic colitis, Diversion colitis, Behçet's syndrome, Indeterminate colitis, Graft versus Host Disease (GvHD), Eaton-Lambert syndrome, Goodpasture's syndrome, Greave's disease, Guillain-Barr syndrome, autoimmune hemolytic anemia (AIHA), hepatitis, insulin-dependent diabetes mellitus (IDDM) and NIDDM, multiple sclerosis (MS), myasthenia gravis, plexus disorders e.g. acute brachial neuritis, polyglandular deficiency syndrome, primary biliary cirrhosis, scleroderma, thrombocytopenia, thyroiditis e.g. Hashimoto's disease, Sjogren's syndrome, allergic purpura, psoriasis, mixed connective tissue disease, polymyositis, dermatomyositis, vasculitis, polyarteritis nodosa, arthritis, alopecia areata, polymyalgia rheumatica, Wegener's granulomatosis, Reiter's syndrome, ankylosing spondylitis, pemphigus, bullous pemphigoid, dermatitis herpetiformis, psoriatic arthritis, reactive arthritis, and ankylosing spondylitis, inflammatory arthritis, including juvenile idiopathic arthritis, gout and pseudo gout, as well as arthritis associated with colitis or psoriasis, Pernicious anemia, some types of myopathy and Lyme disease (Late).

In some embodiments, the immune-related disorder applicable in the methods of the present disclosure may be an immunodeficiency. Immunodeficiency (or immune deficiency) is a state in which the immune system's ability is compromised or entirely absent. Most cases of immunodeficiency are acquired (“secondary”) due to extrinsic factors that affect the patient's immune system. Examples of these extrinsic factors include viral infection, specifically, HIV, extremes of age, and environmental factors, such as nutrition. In the clinical setting, the immunosuppression by some drugs, such as steroids, can be either an adverse effect or the intended purpose of the treatment. Examples of such use are in organ transplant surgery as an anti-rejection measure and in patients suffering from an overactive immune system, as in autoimmune diseases. Immunodeficiency also decreases cancer immuno-surveillance, in which the immune system scans the cells and kills neoplastic ones. Still further, Primary immunodeficiencies (PID), also termed innate immunodeficiencies, are disorders in which part of the organism immune system is missing or does not function normally. To be considered a primary immunodeficiency, the cause of the immune deficiency must not be related to other disease, drug treatment, or environmental exposure to toxins. Most of the PIDs are genetic disorders. Secondary immunodeficiencies occur when the immune system is compromised due to environmental factors. Such factors include but are not limited to chemotherapy, radiotherapy, biological therapy, bone marrow transplantation, gene therapy, adoptive cell transfer or any combinations thereof. In some embodiments, the pathologic disorder applicable in the methods of the present disclosure may be at least one neurodegenerative disorder. Neurodegeneration is the umbrella term for the progressive loss of structure or function of neurons, including synaptic dysfunction and death of neurons. Many neurodegenerative diseases including Parkinson's and Alzheimer's are associated with neurodegenerative processes. Other examples of neurodegeneration that may be also applicable herein may include Friedreich's ataxia, Lewy body disease, spinal muscular atrophy, multiple sclerosis, frontotemporal dementia, corticobasal degeneration, progressive supranuclear palsy, multiple system atrophy, hereditary spastic paraparesis, amyloidosis, Amyotrophic lateral sclerosis (ALS), and Charcot Marie Tooth. It should not be overlooked that normal aging processes include progressive neurodegeneration, specifically, age-related cognitive decline (ACD) and mild cognitive impairment (MCI) are also applicable in the present disclosure. In some embodiments, the physiological state and/or condition of a subject is a behavioral/mental state and/or condition. “Behavioral/mental state” as used herein, reflects the state of mind of a subject or refers to reactions made by a subject in response to stimuli from the immediate environment, and/or past experiences, and/or internal factors. Still further, in some embodiments, the behavioral/mental state and/or condition may be any cognitive and/or behavioral/mental disorder. A cognitive and/or behavioral/mental disorder is characterized by a clinically significant disturbance in an individual's cognition, emotional regulation, or behavior. It is usually associated with distress or impairment in important areas of functioning. Mental disorders may also be referred to as mental health conditions. The latter is a broader term covering mental disorders, psychosocial disabilities and (other) mental states associated with significant distress, impairment in functioning, or risk of self-harm. There are many different types of mental disorders including for example anxiety disorders, depression, bipolar disorder, post-traumatic stress disorder (PTSD), schizophrenia, eating disorders, such as anorexia nervosa and bulimia nervosa, disruptive behavior and dissocial disorders and neurodevelopmental disorders such as autism spectrum disorder (ASD), and attention deficit hyperactivity disorder (ADHD) amongst others. In some further embodiments, the physiological state and/or condition of a subject is a metabolic state. A “metabolic state”, as used herein, refers to the set of chemical reactions that occur in living organisms. Still further, in some embodiments, the metabolic state may be at least one metabolic disorder. Metabolic disorders may include atherosclerosis and peripheral vascular diseases, as well as cardiovascular diseases such as coronary artery diseases (CAD). Of particular interest in connection with metabolic disorders are conditions associated with obesity, hypertension, elevated cholesterol (combined hyperlipidemia), such conditions often termed metabolic syndrome (it is also known as Syndrome X, Reavan's syndrome, or CHAOS). It should be noted that the disclosed conditions may be congenital or acquired conditions.

In some embodiments, the immune-related disorder used by the present disclosure refers to a condition involving at least one wound in at least one tissue and/or organ of a subject. A wound is any disruption of or damage to living tissue, such as skin, mucous membranes, or organs. Wounds can either be the sudden result of direct trauma (mechanical, thermal, chemical), or can develop slowly over time due to underlying disease processes such as diabetes mellitus, venous/arterial insufficiency, or immunologic disease. In certain specific embodiments, the disclosed method may be applicable for inflammatory conditions such as inflammatory bowel disease (IBD). In some specific embodiments, an inflammatory bowel disease (IBD) diagnosed by the disclosed methods in a subject may be Crohn's disease (CD). In yet some further embodiments, an inflammatory bowel disease (IBD) diagnosed by the disclosed methods in a subject may be ulcerative colitis (UC). Inflammatory bowel disease (IBD) is characterized by repetitive episodes of inflammation of the gastrointestinal tract caused by an abnormal immune response to gut microflora. Inflammatory bowel disease encompasses two types of idiopathic intestinal disease that are differentiated by their location and depth of involvement in the bowel wall. Ulcerative colitis (UC) involves diffuse inflammation of the colonic mucosa. Most often UC affects the rectum (proctitis), but it may extend into the sigmoid (proctosigmoiditis), beyond the sigmoid (distal ulcerative colitis), or include the entire colon up to the cecum (pancolitis). Crohn disease (CD) results in transmural ulceration of any portion of the gastrointestinal tract (GI) most often affecting the terminal ileum and colon. Both diseases are classified by extent (mild, moderate, or severe) and location. CD also is classified by phenotype-inflammatory, stricturing, or penetrating. Besides the GI tract, both Crohn disease and ulcerative colitis have many extraintestinal manifestations, all are applicable for the methods of the present disclosure. In yet some further embodiments, the methods of the preset disclosure may be applicable for at least one proliferative disorder. In some specific embodiments, such proliferative disorder may be at least one malignant neoplastic disorder. As used herein to describe the present disclosure, “proliferative disorder”, “malignant neoplastic disorder”, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. Malignancy, as contemplated in the present disclosure may be any one of melanomas, carcinomas, lymphomas, leukemia, myeloma and sarcomas. In yet some further embodiments, the methods of the present disclosure may be applicable for diagnosing a malignant neoplastic disorder such as melanoma. Melanoma as used herein, is a malignant tumor of melanocytes. Melanocytes are cells that produce the dark pigment, melanin, which is responsible for the color of skin. They predominantly occur in skin but are also found in other parts of the body, including the bowel and the eye. Melanoma can occur in any part of the body that contains melanocytes. In some embodiments, the methods of the present disclosure may be applicable for any solid tumor. In more specific embodiments, the methods disclosed herein may be applicable for any malignancy that may affect any organ or tissue in any body cavity, for example, the peritoneal cavity (e.g., liposarcoma), the pleural cavity (e.g., mesothelioma, invading lung), any tumor in distinct organs, for example, the urinary bladder, ovary carcinomas, and tumors of the brain meninges. It should be understood that the methods of the present disclosure are applicable for any type and/or stage and/or grade of any of the malignant disorders discussed herein or any metastasis thereof. Still further, it must be appreciated that the methods of the present disclosure may be applicable for invasive as well as non-invasive cancers. When referring to “non-invasive” cancer it should be noted as a cancer that do not grow into or invade normal tissues within or beyond the primary location. When referring to “invasive cancers” it should be noted as cancer that invades and grows in normal, healthy adjacent tissues.

Still further, in some embodiments, the methods, and kits of the present disclosure are applicable for any type and/or stage and/or grade of any metastasis, metastatic cancer or status of any of the cancerous conditions disclosed herein. More specifically, further malignancies that may find utility in the present disclosure can comprise but are not limited to hematological malignancies (including lymphoma, leukemia, myeloproliferative disorders, Acute lymphoblastic leukemia; Acute myeloid leukemia), hypoplastic and aplastic anemia (both virally induced and idiopathic), myelodysplastic syndromes, all types of paraneoplastic syndromes (both immune mediated and idiopathic) and solid tumors (including GI tract, colon, lung, liver, breast, prostate, pancreas and Kaposi's sarcoma. The present disclosure may be applicable as well for the treatment or inhibition of solid tumors such as tumors in lip and oral cavity, pharynx, larynx, paranasal sinuses, major salivary glands, thyroid gland, esophagus, stomach, small intestine, colon, colorectum, anal canal, liver, gallbladder, extraliepatic bile ducts, ampulla of vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma, Adrenocortical carcinoma; AIDS-related cancers; AIDS-related lymphoma; Anal cancer; Appendix cancer; Astrocytoma, childhood cerebellar or cerebral; Basal cell carcinoma; Bile duct cancer, extrahepatic; Bladder cancer; Bone cancer, Osteosarcoma/Malignant fibrous histiocytoma; Brainstem glioma; Brain tumor; Brain tumor, cerebellar astrocytoma; Brain tumor, cerebral astrocytoma/malignant glioma; Brain tumor, ependymoma; Brain tumor, medulloblastoma; Brain tumor, supratentorial primitive neuroectodermal tumors; Brain tumor, visual pathway and hypothalamic glioma; Breast cancer; Bronchial adenomas/carcinoids; Burkitt lymphoma; Carcinoid tumor, childhood; Carcinoid tumor, gastrointestinal; Carcinoma of unknown primary; Central nervous system lymphoma, primary; Cerebellar astrocytoma, childhood; Cerebral astrocytoma/Malignant glioma, childhood; Cervical cancer; Childhood cancers; Chronic lymphocytic leukemia; Chronic myelogenous leukemia; Chronic myeloproliferative disorders; Colon Cancer; Cutaneous T-cell lymphoma; Desmoplastic small round cell tumor; Endometrial cancer; Ependymoma; Esophageal cancer; Ewing's sarcoma in the Ewing family of tumors; Extracranial germ cell tumor, Childhood; Extragonadal Germ cell tumor; Extrahepatic bile duct cancer; Eye Cancer, Intraocular melanoma; Eye Cancer, Retinoblastoma; Gallbladder cancer; Gastric (Stomach) cancer; Gastrointestinal Carcinoid Tumor; Gastrointestinal stromal tumor (GIST); Germ cell tumor: extracranial, extragonadal, or ovarian; Gestational trophoblastic tumor; Glioma of the brain stem; Glioma, Childhood Cerebral Astrocytoma; Glioma, Childhood Visual Pathway and Hypothalamic; Gastric carcinoid; Hairy cell leukemia; Head and neck cancer; Heart cancer; Hepatocellular (liver) cancer; Hodgkin lymphoma; Hypopharyngeal cancer; Hypothalamic and visual pathway glioma, childhood; Intraocular Melanoma; Islet Cell Carcinoma (Endocrine Pancreas); Kaposi sarcoma; Kidney cancer (renal cell cancer); Laryngeal Cancer; Leukemias; Leukemia, acute lymphoblastic (also called acute lymphocytic leukemia); Leukemia, acute myeloid (also called acute myelogenous leukemia); Leukemia, chronic lymphocytic (also called chronic lymphocytic leukemia); Leukemia, chronic myelogenous (also called chronic myeloid leukemia); Leukemia, hairy cell; Lip and Oral Cavity Cancer; Liver Cancer (Primary); Lung Cancer, Non-Small Cell; Lung Cancer, Small Cell; Lymphomas; Lymphoma, AIDS-related; Lymphoma, Burkitt; Lymphoma, cutaneous T-Cell; Lymphoma, Hodgkin; Lymphomas, Non-Hodgkin (an old classification of all lymphomas except Hodgkin's); Lymphoma, Primary Central Nervous System; Marcus Whittle, Deadly Disease; Macroglobulinemia, Waldenstrom; Malignant Fibrous Histiocytoma of Bone/Osteosarcoma; Medulloblastoma, Childhood; Melanoma; Melanoma, Intraocular (Eye); Merkel Cell Carcinoma; Mesothelioma, Adult Malignant; Mesothelioma, Childhood; Metastatic Squamous Neck Cancer with Occult Primary; Mouth Cancer; Multiple Endocrine Neoplasia Syndrome, Childhood; Multiple Myeloma/Plasma Cell Neoplasm; Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple (Cancer of the Bone-Marrow); Myeloproliferative Disorders, Chronic; Nasal cavity and paranasal sinus cancer; Nasopharyngeal carcinoma; Neuroblastoma; Non-Hodgkin lymphoma; Non-small cell lung cancer; Oral Cancer; Oropharyngeal cancer; Osteosarcoma/malignant fibrous histiocytoma of bone; Ovarian cancer; Ovarian epithelial cancer (Surface epithelial-stromal tumor); Ovarian germ cell tumor; Ovarian low malignant potential tumor; Pancreatic cancer; Pancreatic cancer, islet cell; Paranasal sinus and nasal cavity cancer; Parathyroid cancer; Penile cancer; Pharyngeal cancer; Pheochromocytoma; Pineal astrocytoma; Pineal germinoma; Pineoblastoma and supratentorial primitive neuroectodermal tumors, childhood; Pituitary adenoma; Plasma cell neoplasia/Multiple myeloma; Pleuropulmonary blastoma; Primary central nervous system lymphoma; Prostate cancer; Rectal cancer; Renal cell carcinoma (kidney cancer); Renal pelvis and ureter, transitional cell cancer; Retinoblastoma; Rhabdomyosarcoma, childhood; Salivary gland cancer; Sarcoma, Ewing family of tumors; Sarcoma, Kaposi; Sarcoma, soft tissue; Sarcoma, uterine; Sezary syndrome; Skin cancer (nonmelanoma); Skin cancer (melanoma); Skin carcinoma, Merkel cell; Small cell lung cancer; Small intestine cancer; Soft tissue sarcoma; Squamous cell carcinoma-see Skin cancer (nonmelanoma); Squamous neck cancer with occult primary, metastatic; Stomach cancer; Supratentorial primitive neuroectodermal tumor, childhood; T-Cell lymphoma, cutaneous (Mycosis Fungoides and Sezary syndrome); Testicular cancer; Throat cancer; Thymoma, childhood; Thymoma and Thymic carcinoma; Thyroid cancer; Thyroid cancer, childhood; Transitional cell cancer of the renal pelvis and ureter; Trophoblastic tumor, gestational; Unknown primary site, carcinoma of, adult; Unknown primary site, cancer of, childhood; Ureter and renal pelvis, transitional cell cancer; Urethral cancer; Uterine cancer, endometrial; Uterine sarcoma; Vaginal cancer; Visual pathway and hypothalamic glioma, childhood; Vulvar cancer; Waldenstrom macroglobulinemia and Wilms tumor (kidney cancer).

Still further, the disclosed methods involve analyzing phase variations in specific loci of at least one microorganism in a sample obtained from a subject, or an environmental sample. In some embodiments, such microorganism may be at least one microorganism residing within at least one microbiome community of the subject and/or the media or environmental habitat. A microorganism, or microbe, is an organism of a microscopic size, which may exist in its single-celled form or as a colony of cells. Microorganisms also make up the microbiota found in and on all multicellular organisms. Microorganisms herein refer to bacteria, archaea, fungi, algae, protists, viruses, and bacteriophages. Still further, Microbiome community is a community of commensal, symbiotic, and pathogenic microorganisms (such as bacteria, archea, fungi, viruses, algae, protists and bacteriophages) that can usually be found living together in a particular environment. Human microbiome includes for example gut, skin, genital, and oral microbiome communities. Plant microbiome (also known as the phytomicrobiome) includes for example the rhizosphere (the 1-10 mm zone of soil immediately surrounding the roots that is under the influence of the plant through its deposition of root exudates, mucilage and dead plant cells), the phyllosphere (aerial surface of a plant such as stem, leaf, flower and fruit) and the endosphere microbiome (which penetrate and occupy the plant internal tissues). Environmental microbiome communities include air, soil and aquatic microbial ecology in different habitats. Still further, in some embodiments, the microbiome may be the gut microbiome. Thus, the microorganisms analyzed for phase variation by the disclosed methods may be any microorganism residing in the gut microbiome. “Gut microbiota”, gut microbiome, or gut flora, are the microorganisms, including bacteria, archaea, fungi, and viruses that live in the digestive tracts of animals. It should be understood however that microorganisms of any habitat and/or microbiome may be used in the methods of the present disclosure, for example, the skin microbiome, the genital microbiome, the oral microbiome etc. In some embodiments, the gut microbiome may comprise at least one of: bacteria, archaea, fungi, algae, protists, viruses, and bacteriophages. Thus, at least one microorganism analyzed for phase variations by the methods of the present disclosure may be any bacteria, archaea, fungi, algae, protists, viruses, bacteriophages and/or any combinations thereof. “Bacteria”, as used herein, include Gram positive, Gram negative and Gram variable bacteria and intracellular bacteria. The term “archaea”, as used herein, refers to one of the three domains of living organisms: Archaea, Bacteria and Eukaryota. Archaea include metabolic oddities, methanogens and sulphur-dependent extreme thermophiles. The term “fungi” (or a “fungus”), as used herein, refers to a division of eukaryotic unicellular organisms including for example, fungi that cause diseases such as ringworm, histoplasmosis, blastomycosis, aspergillosis, cryptococcosis, sporotrichosis, coccidioidomycosis, paracoccidio-idoinycosis, and candidiasis. The term “algae”, as used herein, refers to a group of plants living in the water, including all seaweeds. “Protists” are any eukaryotic organism that is not an animal, plant, or fungus. Protists do not form a natural group, or clade, but an artificial grouping of several independent clades that evolved from the last eukaryotic common ancestor. “Viruses” are infectious agents that replicates only inside the living cells of an organism. As used herein this term encompasses enveloped or naked, DNA or RNA, single strand or double strand viruses of any family or genera, for example, poxviruses, herpesviruses, picornaviruses, parvoviruses, hepadnaviruses, picornaviruses, flaviviruses, retroviruses, hepadnaviruses, coronaviruses, arenaviruses, bunyaviruses, and the like. A “bacteriophage”, also known informally as a phage, is a duplodnaviria virus that infects and replicates within bacteria and archaea. Still further, in some embodiments, the methods of the preset disclosure involve analyzing phase variation in specific loci in bacteria. In more specific embodiments, bacteria, as disclosed herein comprise at least one bacterium of at least one phylum selected from Bacteroidota, Verrucomicrobiota, proteobacteria, actinobacteria, firmicutes and Tenericutes. The phylum “Bacteroidota” (synonym Bacteroidetes) is composed of three large classes of Gram-negative, nonsporeforming, anaerobic or aerobic, and rod-shaped bacteria that are widely distributed in the environment, including in soil, sediments, and sea water, as well as in the guts and on the skin of animals. In some embodiments Bacteroidota may comprise Bacteroides fragilis or any species or isolate thereof. “Verrucomicrobiota” is a phylum of Gram-negative bacteria that contains only a few described species, which have been isolated from fresh water, marine and soil environments and human feces. This phylum is considered to have two sister phyla: Chlamydiota (formerly Chlamydiae) and Lentisphaerota (formerly Lentisphaerae) within the PVC superphylum, all are encompassed by the present disclosure. In some embodiments, Verrucomicrobiota may comprise Akkermansia muciniphila, or any species or isolate thereof. Still further, “Proteobacteria”, also called Pseudomonadota is a major phylum of Gram-negative bacteria, which includes a wide variety of pathogenic genera, such as Escherichia, Salmonella, Vibrio, Yersinia, Legionella, and many others. “Actinobacteria” also called Actinomycetota are a diverse phylum of Gram-positive bacteria with high G+C content found in soil. “Firmicutes” also called Bacillota are a phylum of bacteria, most of which have gram-positive cell wall structure, and they are all defined as the core group of related forms called the low-G+C group, in contrast to the Actinomycetota. “Tenericutes” or Mycoplasmatota is a phylum of gram-negative bacteria consisting of cells bounded by a plasma membrane, and they are devoid of cell walls. This phylum contains the class Mollicutes. Notable genera that may be applicable in the present disclosure, include Mycoplasma, Spiroplasma, Ureaplasma, and Candidatus Phytoplasma. In more specific embodiments, Bacteroidota bacterium appliable in the disclosed methods may be any bacteria of the genus Bacteroides, Bacteroidia, Bacteroidales, Bacteroidaceae and Phocaeicola, and any combinations thereof. “Bacteroides” is a genus of Gram-negative, obligate anaerobic bacteria. Bacteroides species are non endospore-forming bacilli, and their membranes contain sphingolipids, and also meso-diaminopimelic acid in their peptidoglycan layer. Bacteroides species form the most substantial portion of the mammalian gastrointestinal microbiota. In yet some further embodiments, bacteria analyzed for phase variations in the methods of the preset disclosure may comprises at least one of Bacteroides fragilis, Bacteroides thetaiotaomicron, Phocaeicola dorei, Bacteroides cellulosilyticus, Bacteroides ovatus, Bacteroides stercorirosoris, or any isolate or species thereof.

In some embodiments, bacterial isolates or species applicable in the disclosed method may comprise Phocaeicola dorei isolate MGYG-HGUT-02478, Bacteroides stercorirosoris strain DSM 26884 and Bacteroides cellulosilyticus strain WH2, and the like. Still further, in some embodiments, any Bacteroidota bacteria may be useful in the disclosed methods, such Bacteroidota bacteria may comprise in some embodiments, at least one of Bacteroides fragilis, Bacteroides uniformis, Bacteroides ovatus, Bacteroides stercoris, Bacteroides cellulosilyticus, Bacteroides caccae, Bacteroides eggerthii, Bacteroides thetaiotaomicron, Bacteroides intestinalis, Bacteroides clarus, Bacteroides fragilis_A, Bacteroides finegoldii, Bacteroides faecis, Bacteroides massiliensis, Bacteroides togonis, Bacteroides nordii, Bacteroides salyersiae, Bacteroides intestinalis_A, Bacteroides ndongoniae, Bacteroides sp003545565, Bacteroides sp905207245, Bacteroides bouchesdurhonensis, Bacteroides fluxus, Bacteroides gallinarum, Bacteroides stercorirosoris, Bacteroides graminisolvens, Bacteroides pyogenes, Bacteroides oleiciplenus, Bacteroides sp002491635, Bacteroides cutis, Bacteroides sp900547205, Bacteroides acidifaciens, Bacteroides sp905197435, Bacteroides neonati, Bacteroides sp014385165 and/or Parabacteroides distasonis, and any species and isolates thereof. In yet some embodiments, Verrucomicrobiota bacteria may be analyzed for phase variations by the disclosed methods. In some embodiments, Verrucomicrobiota may comprise Akkermansia muciniphila, or any species or isolate thereof. Still further, in some embodiments, Bacteroides fragilis, or any species or isolates thereof, may be useful in the disclosed methods. Bacteroides fragilis is an anaerobic, Gram-negative, pleomorphic to rod-shaped bacterium. It belongs to the Bacteroides genus, and Bacteroidota phylum. Bacteroides fragilis resides in the human gastrointestinal tract and is essential to healthy gastrointestinal function such as mucosal immunity and host nutrition. B. fragilis utilizes a complex series of surface proteins, lipopolysaccharide chains, and outer membrane vesicles. B. fragilis can also modulate its surface by expressing different polysaccharides, particularly, B. fragilis polysaccharide A (PSA) can modulate the host immune system. More specifically, PSA was shown to confer protection against experimental colitis, and thus is regarded as an anti-inflammatory polysaccharide.

Still further, in some embodiments, Bacteroides thetaiotaomicron, Phocaeicola dorei, and Bacteroides cellulosilyticus or any species or isolates thereof, may be useful in the disclosed methods. Specifically, “Bacteroides thetaiotaomicron” is a gram-negative, rod shaped obligate anaerobic bacterium that is a prominent member of the normal gut microbiome in the distal intestines. “Phocaeicola dorei” is a gram negative, rod-shaped bacteria that found and contributes to normal intestinal functionality. Phocaeicola dorei is a non-spore-forming, non-motile, and anaerobic bacterium with a G+C DNA content of 43%. “Bacteroides cellulosilyticus” (identified as strain CRE21) are a species of bacteria within the Bacteroides genus, degrading cellulose (cellulolytic) within the human microbiota. Its genome is characterized with G-C content of 43.05%. Still further, in the disclosed method any isolate or species of Bacteroides fragilis, Bacteroides thetaiotaomicron, Phocaeicola dorei, Bacteroides cellulosilyticus, Bacteroides ovatus, Bacteroides stercorirosoris, may be applicable. The term “isolate” or a “genetic isolate” refers to a population of organisms with little genetic mixing with other organisms within the same species due to geographic isolation or other factors that prevent reproduction. The term “species” (pl. species) is the basic unit of classification and a taxonomic rank of an organism, defined by their karyotype, DNA sequence, morphology, behavior, or ecological niche. As indicated above, the disclosed methods involve determining phase variation in at least one locus of at least one microorganism (e.g., bacteria) in at least one sample. In some embodiments, phase variation/s determined by the disclosed methods may comprise any phase variations in at least one intergenic region/s and/or intragenic region/s.

A locus (plural loci) as used herein is a specific, fixed position on a chromosome where a particular nucleic acid sequence or a gene is located. In some embodiments, the phase variation may be determined in intergenic regions. Specifically, intergenic regions are a stretch of DNA sequences located between genes, and include promoters, enhancers, and other regulatory elements, origins of replication, transposons and viruses. Non-functional DNA elements include for example pseudogenes and repetitive DNA, both of which are types of junk DNA. In yet some further embodiments, the phase variation may be determined in intergenic regions that are a stretch of DNA sequences located within genes. In some embodiments, the methods of the present disclosure involve the step of determining phase variation in at least one locus in bacteria. In some embodiments, the at least one locus may comprise nucleic acid sequence/s encoding and/or regulating at least one outersurface and/or internal molecule and/or at least one molecule that modify or regulate the at least one outersurface and/or internal molecule/s. “Outersurface” molecules refer herein to molecules which reside within the cell surface of an organism and/or at the outside part of the cell surface of an organism. “Internal” molecules refer herein to molecules which reside inside the cell, in the internal area of an organism's cell. The locus determined for phase variations may comprise, in some embodiments, a sequence that modify or regulate the outer or inner membrane, specifically, a sequence that changes directly or indirectly the activity, stability, post translational modifications, of the outer or inner molecule, thereby, defining the term “modify” as used herein.

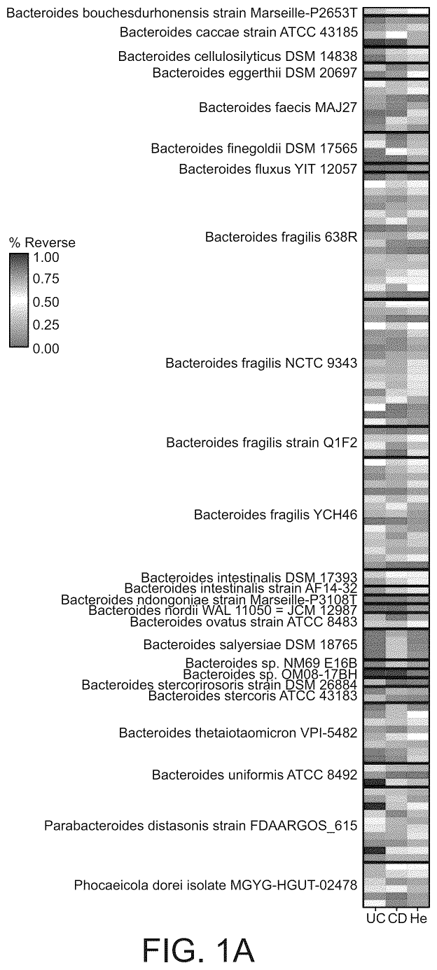

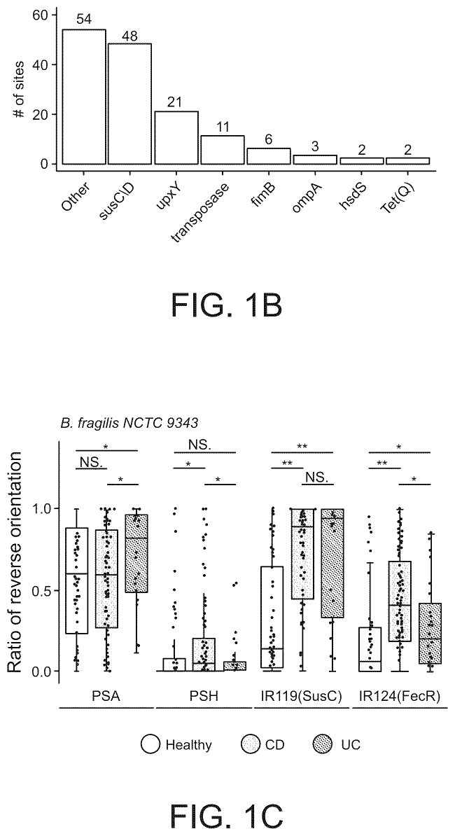

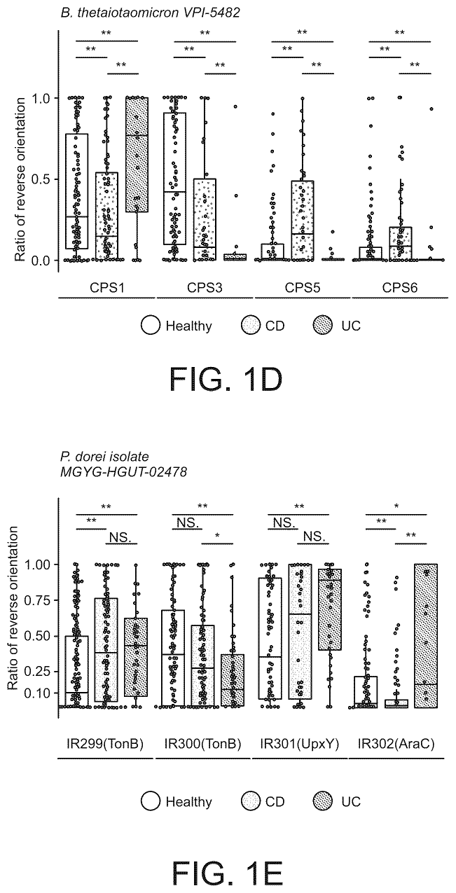

In some embodiments, the phase variations of at least one microorganism (e.g., bacteria) in at least one sample of the diagnosed subject or media and/or habitat, are analyzed and determined in at least one locus. In some embodiments, such at least one locus may comprise at least one of polysaccharide utilization loci (PUL), specifically, SusC/D, capsular polysaccharide (CPS), ribosomal RNA (rRNA) 23S, rRNA 16S, fimbria, transposase, HsdS, fimB, outer membrane protein A (OmpA), Tetracycline resistance protein (Tet(Q) and Transfer RNA (tRNA), helix-turn-helix transcriptional regulator or DUF6198 family protein, HAD family phosphatase or MATE family efflux transporter, ATP-binding protein, UpxY family transcription antiterminator and/or TonB-dependent receptor, and any combinations thereof. More specifically, “Ribosomal ribonucleic acid (rRNA)” is non-coding RNA forming the primary component of ribosomes. rRNA is a ribozyme involved in protein synthesis in ribosomes. Ribosomal RNA comprises two major ribosomal subunit: the large subunit (LSU) and the small subunit (SSU), forms together a functioning ribosome. In prokaryotes, the LSU and SSU are called the 50S and 30S subunits, respectively. There are three types of rRNA found in prokaryotic ribosomes: “23S rRNA” and 5S rRNA in the LSU and “16S rRNA” in the SSU. “Fimbriae”, also referred to as pili, mediate adhesion of the bacterial cell to host tissue through interaction with receptors located on the host cell. A “transposase”, as used herein, is any of a class of enzymes capable of binding to the end of a transposon and catalyzing its movement to another part of a genome, typically by a cut-and-paste mechanism or a replicative mechanism, in a process known as transposition. Still further, “Hydroxysteroid dehydrogenases (HSDs)”, are a group of alcohol oxidoreductases that catalyze the dehydrogenation of hydroxysteroids. These enzymes also catalyze the reverse reaction, acting as ketosteroid reductases (KSRs). There are several types of HSDs, including 3α-HSD, 3β-HSD, 11β-HSD, 17β-HSD and 20β-HSD, applicable in the present disclosure. “Outer membrane protein A (OmpA)” is a multi-functional major outer membrane protein of Escherichia coli and other Enterobacteriaceae family, that also serves as a receptor for several bacteriocin and bacteriophages. Still further, “Tet(Q)” is a tetracycline resistance ribosomal protection protein. Its gene is associated with a conjugative transposon and has been found in both Gram-positive and Gram-negative bacteria. “Transfer RNA” (abbreviated “tRNA”) is an adaptor molecule composed of RNA, that serves as the physical link between the mRNA and the amino acid sequence of proteins. Still further, the locations of the inverted repeats (IR) sequences and genes flanking the phase variable regions are summarized in FIG. 1A. More specifically, in some embodiment, IRs that that were shown by the present disclosure in the following various loci in the various bacteria genomes indicated herein, may be used by the present disclosure for determining phase variations: Bacteroides bouchesdurhonensis Marseille-P2653T:NZ_LT707006.1:1647805_1648389, NZ_LT707006.1:1649135_1649475, NZ_LT707006.1:3646095_3646363, NZ_LT707006.1:3906426_3906529, NZ_LT707006.1:4046762_4047102, NZ_LT707006.1:4085306_4086045, NZ_LT707006.1:4088570_4089283, NZ_LT707006.1:4605941_4606027, NZ_LT707006.1:4606820_4607160, NZ_LT707006.1:4968747_4969087, NZ_LT707006.1:4973158_4973251, NZ_LT707006.1:5047636_5047976, NZ_LT707006.1:5048399_5048492. Bacteroides caccae ATCC 43185: NZ_CP022412.2:41071_41422, NZ_CP022412.2:513789_514146, NZ_CP022412.2:1584595_1585156, NZ_CP022412.2:1922216_1922661, NZ_CP022412.2:1936310_1936714, NZ_CP022412.2:3172369_3172667, NZ_CP022412.2:3654461_3654753, NZ_CP022412.2:3899824_3900257, NZ_CP022412.2:4162323_4162633, NZ_CP022412.2:4399881_4400047, NZ_CP022412.2:4475452_4475804, NZ_CP022412.2:4553478_4553692.

Bacteroides cellulosilyticus DSM 14838:
NZ_EQ973492.1:91545_91672, NZ_EQ973490.1:106903_107231, NZ_EQ973488.1:256707_256928, NZ_EQ973491.1:264957_265029, NZ_EQ973491.1:270611_270817, NZ_EQ973490.1:315816_315906, NZ_EQ973490.1:1317757_1318244, NZ_EQ973491.1:1347761_1348084, NZ_EQ973490.1:2126168_2126280.
   Bacteroides clarus YIT 12056:

NZ_FQWK01000004.1:51678_51761, NZ_FQWK01000002.1:63300_63427, NZ_FQWK01000006.1:228672_228893, NZ_FQWK01000004.1:306713_307056, NZ_FQWK01000003.1:315979_316644, NZ_FQWK01000004.1:364215_364904. Bacteroides cutis Marseille-P4118: NZ_OEST01000001.1:318543_319218. Bacteroides eggerthii DSM 20697: NZ_DS995511.1:57933_58159, NZ_DS995509.1:200342_200440, NZ_DS995511.1:511092_511168, NZ_DS995511.1:612486_612699, NZ_DS995510.1:743833_744056, NZ_DS995509.1:1046254_1046482, NZ_DS995510.1:1085619_1085853. Bacteroides faecis MAJ27: NZ_FNNN01000010.1:8174_8788, NZ_FNNN01000050.1:14813_14948, NZ_FNNN01000014.1:24280_24567, NZ_FNNN01000019.1:24289_24587, NZ_FNNN01000002.1:53187_53900, NZ_FNNN01000029.1:59477_59820, NZ_FNNN01000021.1:59683_59972, NZ_FNNN01000028.1:60669_61103, NZ_FNNN01000004.1:71435_71711, NZ_FNNN01000012.1:80581_80680, NZ_FN NN01000015.1:120613_120931_, NZ_FNNN01000002.1:168531_168614, NZ_FNNN01000003.1:182061_182144, NZ_FNNN01000002.1:210574_210788, NZ_FNNN01000005.1:212430_212720, NZ_FNNN01000003.1:221774_221857, NZ_FNNN01000001.1:291601_291945. Bacteroides finegoldii DSM 17565: NZ_GG688341.1:15256_15357, NZ_GG688343.1:27175_27651, NZ_GG688340.1:30892_31193, NZ_GG688335.1:37098_37841, NZ_GG688336.1:52110_52193, NZ_GG688332.1:54294_54391, NZ_GG688323.1:68844_68990, NZ_GG688328.1:107477_107558, NZ_GG688319.1:120964_121593, NZ_GG688326.1:186634_186931, NZ_GG688319.1:187 444_187518, NZ_GG688319.1:192810_193005, NZ_GG688319.1:331326_331506, NZ_GG688318.1:349475_349612, NZ_GG688317.1:442893_443261. Bacteroides fluxus YIT 12057: NZ_GL882693.1:87395_87613_87395_87421. Bacteroides fragilis 638R: NC_016776.1:79872_80032, NC_016776.1:658830_659165, NC_016776.1:945239_945452, NC_016776.1:1167437_1167835, NC_016776.1:1418088_1418825, NC_016776.1:1421350_1422059, NC_016776.1:1501757_1501989NC_016776.1:1695261_1695491, NC_016776.1:1804132_1804368, NC_016776.1:2203057_2203275, NC_016776.1:2275298_2275590, NC_016776.1:3152073_3152178, NC_016776.1:3239047_3239143, NC_016776.1:3277102_3277474, NC_016776.1:3438538_3438634, NC_016776.1:3452455_3452928, NC_016776.1:3760262_3760362, NC_016776.1:3887616_3888005, NC_016776.1:4251851_4252252, NC_016776.1:4441496_4441732, NC_016776.1:4645324_4645516, NC_016776.1:4880240_4880747, NC_016776.1:4952863_4953023, NC_016776.1:4998058_4998148, NC_016776.1:5110268_5110690, NC_016776.1:5116641_5117074, NC_016776.1:5132693_5133150, NC_016776.1:5151896_5151991_5151896, NC_016776.1:5261037_5261536, NC_016776.1:5287302_5287619. Bacteroides fragilis NCTC 9343: NC_003228.3:603196_603531, NC_003228.3:894511_894724, NC_003228.3:1115334_1115734, NC_003228.3:1442460_1442692, NC_003228.3:1634575_1634805, NC_003228.3:1806789_1807025, NC_003228.3:2211236_2211454, NC_003228.3:2282840_2283132, NC_003228.3:3032390_3032595, NC_003228.3:3116123_3116218, NC_003228.3:3239704_3240076, NC_003228.3:3406827_3406923, NC_003228.3:3420744_3421217, NC_003228.3:3466134_3466251, NC_003228.3:3790067_3790365, NC_003228.3:3839227_3839616, NC_003228.3:3850690_3850836, NC_003228.3:4091660_4091889, NC_003228.3:4218300_4218701, NC_003228.3:4361354_4361586, NC_003228.3:4596176_4596368, NC_003228.3:4653929_4654539, NC_003228.3:4866094_4866254, NC_003228.3:4868436_4868596, NC_003228.3:4911286_4911376, NC_003228.3:5023725_5024147, NC_003228.3:5030098_5030531, NC_003228.3:5046156_5046613, NC_003228.3:5065357_5065452, NC_003228.3:5097961_5098469, NC_003228.3:5124227_5124544. Bacteroides fragilis Q1F2: NZ_CP018937.1:851694_851932, NZ_CP018937.1:1063463_1064017, NZ_CP018937.1:1479169_1479387, NZ_CP018937.1:1617203_1617320, NZ_CP018937.1:2056490_2057023, NZ_CP018937.1:2080479_2080580, NZ_CP018937.1:2157498_2157703, NZ_CP018937.1:2600478_2600938, NZ_CP018937.1:2968801_2968893, NZ_CP018937.1:3102148_3102447, NZ_CP018937.1:3151245_3151635, NZ_CP018937.1:3547498_3547908, NZ_CP018937.1:3741699_3741931, NZ_CP018937.1:3864534_3864827, NZ_CP018937.1:4008455_4008778, NZ_CP018937.1:4186637_4186742, NZ_CP018937.1:4190135_4190225, NZ_CP 018937.1:4304197_4304639, NZ_CP018937.1:4422137_4422297, NZ_CP018937.1:4568851_45 69021. Bacteroides fragilis YCH46: NC_006347.1:655366_655701, NC_006347.1:955685_955898, NC_006347.1:1176106_1176506, NC_006347.1:1452628_1452860, NC_006347.1:1673256_1673486, NC_006347.1:1776171_1776407, NC_006347.1:2153550_2153768, NC_006347.1:2226606_2226898, NC_006347.1:2712186_2712931, NC_006347.1:2970571_2970776, NC_006347.1:3053294_3053389, NC_006347.1:3140272_3140368, NC_006347.1:3178334_3178738, NC_006347.1:3401147_3401239, NC_006347.1:3515162_3515635, NC_006347.1:3560541_3560658, NC_006347.1:3883245_3883543, NC_006347.1:3932405_3932794, NC_006347.1:4180984_4181213, NC_006347.1:4312364_4312765, NC_006347.1:4474459_4474695, NC_006347.1:4672225_4672417, NC_006347.1:4731410_4732020, NC_006347.1:4932624_4932784, NC_006347.1:4934966_4935126, NC_006347.1:4977826_4977916, NC_006347.1:5090820_5091242, NC_006347.1:5097194_5097627, NC_006347.1:5132453_5132548. Bacteroides gallinarum DSM 18171: NZ_KB894116.1:36347_36572. Bacteroides intestinalis DSM NZ_ABJL02000001.1:13955_14691, 17393: NZ_ABJL02000008.1:324985_325206, NZ_ABJL02000008.1:518099_518307, NZ_ABJL02000008.1:720695_720905, NZ_ABJL02000007.1:1220543_1220738, NZ_ABJL02000008.1:1456629_1456709, NZ_ABJL02000008.1:1507466_1507684, NZ_ABJL02000008.1:1910159_1910482, NZ_ABJL02000008.1:3331865_3332202. Bacteroides intestinalis AF14-32: NZ_QRZF01000013.1:93436_93735, NZ_QRZF01000003.1:369775_369873, NZ_QRZF01000003.1:371352_371675. Bacteroides ndongoniae Marseille-P3108T: NZ_FNVV01000013.1:865141_865252, NZ_FNVV01000014.1:1900348_1901095.Bacteroides nordii JCM 12987: BAJA01000064.1:15429_15769, BAJA01000016.1:22342_22679, BAJA01000001.1:249337_249528. Bacteroides ovatus ATCC 8483: NZ_CP012938.1:812339_812515, NZ_CP012938.1:851299_851478, NZ_CP012938.1:1113560_1113866, NZ_CP012938.1:1410309_1410594, NZ_CP012938.1:2519554_2520125, NZ_CP012938.1:4698369_4698467, NZ_CP012938.1:5376155_5376349, NZ_CP012938.1:5614125_5614230. Bacteroides salyersiae DSM 18765: NZ_KB905467.1:631500_631763. Bacteroides sp. NM69_E16B: NZ_SRYZ01000023.1:6448_6519, NZ_SRYZ01000037.1:7392_7464, NZ_SRYZ 01000071.1:9752_9946, NZ_SRYZ01000042.1:19397_20015. Bacteroides sp. OM08-17BH: NZ_QSTC01000002.1:609_889, NZ_QSTC01000004.1:5757_5887, NZ_QSTC01000007.1:117832_118067. Bacteroides stercorirosoris DSM 26884: NZ_FQZN01000011.1:15702_15914, NZ_FQZN01000021.1:19831_20163, NZ_FQZN01000009.1:119226_119909. Bacteroides stercoris ATCC 43183: NZ_DS499673.1:1246_1868, NZ_DS499664.1:15985_16664, NZ_DS499673.1:128735_128813, NZ_DS499662.1:145693_145879, NZ_DS499676.1:207551_207884, NZ_DS499672.1:270695_270917, NZ_DS499674.1:374817_374908, NZ_DS499674.1:436451_436660, NZ_DS499673.1:481995_482281, NZ_DS499673.1:539698_540057, NZ_DS499674.1:660790_661363. Bacteroides thetaiotaomicron VPI-5482: NC_004663.1:463090_463392, NC_004663.1:735864_736151, NC_004663.1:1068507_1068787, NC_004663.1:1854697_1855005, NC_004663.1:2048014_2048292, NC_004663.1:2124254_2124533, NC_004663.1:2382227_2382352, NC_004663.1:2544857_2545011, NC_004663.1:3062773_3063071, NC_004663.1:5233175_5233370, NC_004663.1:5255325_5255667, NC_004663.1:5600155_5600589, NC_004663.1:5667641_5667929. Bacteroides togonis Marseille-P3166T: NZ_LT670820.1:239351_240016, NZ_LT670820.1:242083_242643, NZ_LT670820.1:350525_350738, NZ_LT670820.1:1128811_1129407, NZ_LT670820.1:1144957_1145131, NZ_LT670820.1:1620777_1620880, NZ_LT670820.1:2676236_2676984, NZ_LT670820.1:3564837_3565542. Bacteroides uniformis ATCC 8492: NZ_DS362219.1:1950_2164, NZ_DS362237.1:63080_63204, NZ_DS362249.1:97921_98129, NZ_DS362233.1:64988_65094, NZ_DS362249.1:142622_142836, NZ_DS362249.1:168505_168588, NZ_DS362246.1:177374_177660, NZ_DS362244.1:190809_191043, NZ_DS362245.1:207338_207673. Mediterranea massiliensis Marseille-P2645T: NZ_LT635832.1:95473_96010, NZ_LT635836.1:193310_193459, NZ_LT635839.1:602433_603068. Parabacteroides distasonis FDAARGOS_615: NZ_CP050956.1:7378_8126, NZ_CP050956.1:30911_31356, NZ_CP050956.1:113729_114080, NZ_CP050956.1:1152694_1153064, NZ_CP050956.1:1274702_1274917, NZ_CP050956.1:1298240_1298455, NZ_CP050956.1:1357820_1358012, NZ_CP050956.1:1941720_1941893, NZ_CP050956.1:1958507_1958701, NZ_CP050956.1:2237762_2238029, NZ_CP050956.1:2283422_2283637, NZ_CP050956.1:2499113_2499320, NZ_CP050956.1:2595300_2595568, NZ_CP050956.1:2648501_2648854, NZ_CP050956.1:2869388_2869756, NZ_CP050956.1:2987355_2987447, NZ_CP050956.1:3230775_3231044, NZ_CP050956.1:3317663_3317909, NZ_CP050956.1:3396041_3396396, NZ_CP050956.1:3526373_3526585, NZ_CP050956.1:3582210_3582353, NZ_CP050956.1:3736964_3737157, NZ_CP050956.1:3780196_3780289, NZ_CP050956.1:4253778_4253948, NZ_CP050956.1:5025072_5025169, NZ_CP050956.1:5102944_5103386. Phocaeicola dorei MGYG-HGUT-02478: NZ_LR699004.1:514152_514340, NZ_LR699004.1:641746_642019, NZ_LR699004.1:862176_862387, NZ_LR699004.1:1538524_1538727, NZ_LR699004.1:1797742_1797947,
NZ_LR699004.1:2794857_2795270, NZ_LR699004.1:3222398_3222607, NZ_LR699004.1:3510721_3510923, NZ_LR699004.1:3796128_3796341, NZ_LR699004.1:3802651_3802963, NZ_LR 699004.1:4446456_4446647. uncultured Bacteroides sp. ERR414351-bin.17: CAJJYX010000135.1:3219_3437, CAJJYX010000109.1:9901_10333.

It should be understood that phase variations in each of the disclosed loci may occur in any region, either coding or non-coding regions of each locus. In some embodiments, phase variations determined by the disclosed methods may occur in non-coding regions of genes, e.g., in promoter regions. According to such embodiments, phase variations in promoter regions may lead to ON\OFF control of transcription from at least one gene, or to election and expression of the transcribed gene, in case of reversed orientation of the promoter occurred by the phase variation, lead to transcription of another gene located in the reversed orientation. In yet some further embodiments, the phase variations determined by the disclosed methods may occur within a coding region and thus may result in truncated gene product (e.g. protein) or in a recombinant, rearranged and/or fused protein product. Thereby affecting the levels, activity, stability of the gene product.

In some embodiments, the phase variations of at least one microorganism (e.g., bacteria) in at least one sample of the diagnosed subject or media and/or habitat, are analyzed and determined in at least one of the polysaccharide utilization loci (PUL), and/or capsular polysaccharide (CPS) loci. Polysaccharide utilization loci (PULs), a unique feature of Bacteriodetes genomes, are clusters of colocalized, coregulated genes, the products of which orchestrate the detection, sequestration, enzymatic digestion, and transport of complex carbohydrates. PULs encode a complement of cell surface glycan-binding proteins (SGBPs), TonB-dependent transporters (TBDTs), carbohydrate-active enzymes (CAZymes) (most frequently glycoside hydrolases (GHs) polysaccharide lyases (PLs) and carbohydrate esterases (CEs) where substrate appropriate), and carbohydrate sensors/transcriptional regulators. PULs also include ancillary enzymes such as proteases, sulfatases, and phosphatases. The most well-studied PUL-encoded glycan-up-take system is the starch utilization system (Sus), which binds, degrades, and imports starch into the cell. This gene cluster of Sus is composed of susRABCDEFG. SusR is an inner membrane-spanning sensor/regulator protein that recognizes maltose, in the periplasm and triggers the rapid upregulation of the sus genes. The outer membrane lipoproteins SusDEF facilitate the binding of starch to the cell surface, and bound starch is then hydrolyzed by the α-amylase SusG. The resulting maltooligosaccharides are shuttled into the periplasm via SusC, a TonB-dependent transporter, and further depolymerized by the neopullulanase SusA and α-glucosidase SusB. FIG. 1B indicates that susC/susD is a major phase varied locus. Still further, bacterial capsular polysaccharides (CPSs) are a diverse class of high molecular weight polysaccharides, that confer protective effects to their bearers against a wide range of environmental pressures, most notably against the immune system during infection of their animal hosts, by hiding cell-surface components that might otherwise elicit host immune response. In Gram-negative bacteria, capsular polysaccharides are often attached to the outer membrane at their reducing end through covalently-linked lipids that are inserted into the lipid bilayer of the membrane, that provides a protective surface layer of water-saturated, high molecular weight polysaccharides. In some embodiments, phase variations determined by the methods of the preset disclosure may be in any locus comprising sequences encoding bacterial outersurface molecules. In some specific embodiments, such loci may comprise CPS. In yet some further embodiments, the CPS loci may comprise polysaccharides (PS) locus. Still further, in some embodiments, the PS loci may comprise polysaccharide A (PSA).

Polysaccharides (PS) are long-chain polymeric carbohydrates composed of monosaccharide units bound together by glycosidic linkages. Microbial polysaccharides are produced by microorganisms such as bacteria, fungi, yeast, and algae. Thus, in some embodiments, the methods of the present disclosure may involve the step of determining phase variation in at least one of the PSA locus, the CPS locus and any regulatory sequences thereof. Polysaccharide A (PSA) is a capsular carbohydrate from the commensal gut bacteria Bacteroides fragilis. PSA exhibits unique immunomodulatory effects that seem to come from the zwitterionic nature of the sugar. PSA is one of the two main polysaccharides (the other is PSB) that compose a capsular polysaccharide complex, which Bacteroides fragilis produces and encases itself with. Still further, in some embodiments, determination of phase variations in accordance with the disclosed methods may comprise at least one inversion in at least one promoter region of at least one gene residing within PSA and/or CPS loci. In yet some further specific and no-limiting embodiments, the methods of the present disclosure may involve the step of determining phase variations in at least one promoter region of at least one gene residing in at least one of the PSA and the CPS loci, such that the phase variation converts the ON/OFF orientation of the at least one promoter region/s. In some embodiments, an inverted orientation of the at least one promoter region results in an OFF status of the promoter, thereby reduces the expression levels of at least one protein product regulated by the promoter. Non-limiting embodiments for such products include the UpaY, and other PSA proteins. Still further, in some other embodiments, the disclosed methods may involve determination of phase variations, specifically, phase variations that comprise at least one inversion in at least one promoter region of at least one gene residing in at least one of the helix-turn-helix transcriptional regulator or DUF6198 family protein, HAD family phosphatase or MATE family efflux transporter, ATP-binding protein, UpxY family transcription antiterminator, TonB-dependent receptor loci, thereby converting the ON/OFF orientation of the at least one promoter region/s. In some alternative embodiments, phase variation in any of these loci may lead to inversion causing reversed orientation of regulatory regions in the specific loci, thereby leading to alternative election of transcribed sequences, as also exemplified by Example 4. More specifically, “Helix-turn-helix” is a DNA-binding protein (DBP). The helix-turn-helix (HTH) is a major structural motif capable of binding DNA. Each monomer incorporates two α helices, joined by a short strand of amino acids, that bind to the major groove of DNA. The HTH motif occurs in many proteins that regulate gene expression.

The “DUF6198 family protein” family, as used herein, represents a putative integral membrane protein that is likely to be the membrane component of an ABC transport (ATP-binding cassette transport) system. The ABC system utilize the energy of adenosine triphosphate (ATP) binding and hydrolysis to provide the energy needed for the translocation of substrates across membranes, either for uptake or for export of a substrate. “Phosphatases of the haloacid dehalogenase (HAD)” superfamily of hydrolases are a very large class of enzymes that have evolved to dephosphorylate substrates with often exquisite specificities. The “MATE” (“Multidrug And Toxic Compound Extrusion”) efflux transport family, as used herein, is one among five multidrug efflux transporter families, known to play an important role in intrinsic and acquired resistance in many bacteria. MATE is a family of proteins which function as drug/sodium or proton antiporters. Still further, “ATP-binding proteins” are proteins which possess an ATP-binding site and are capable of binding ATP. ATP binding sites, are present in many proteins including active membrane transporters, microtubule subunits, flagellum proteins, and various hydrolytic and proteolytic enzymes, all are encompassed by the present disclosure. “UpxY family transcription antiterminator” are involved in the biosynthesis of Bacteroides fragilis polysaccharides. A single strain of Bacteroides fragilis synthesizes eight distinct capsular polysaccharides, designated PSA to PSH, and encoded by eight separate polysaccharide biosynthesis loci. “TonB-dependent receptors”, as used herein, also known as outer membrane receptors, are a family of beta barrel proteins named for their localization in the outer membrane of gram-negative bacteria. TonB complexes sense signals from the outside of bacterial cells and transmit them into the cytoplasm, leading to transcriptional activation of target genes.

In some embodiments, phase variations are determined in the promoter region of PSA of Bacteroides fragilis NCTC 9343, that comprise IRs located at coordinates NC_003228.3:1634575_1634593_1634787_1634805, of the promoter as denoted by SEQ ID NO: 9 (nucleotides 38-56 and 249-268). In some embodiments, phase variations are determined in the promoter region of PSH of Bacteroides fragilis NCTC 9343, that comprise IRs located at coordinates NC_003228.3:894511_894530_894705_894724, of the promoter as denoted by SEQ ID NO: 10 (nucleotides 26-44 and 237-255). In some embodiments, phase variations are determined in the promoter region of IR119 of Bacteroides fragilis NCTC 9343, that comprise IRs located at coordinates NC_003228.3:2282840_2282859_2283113_2283132, of the promoter as denoted by SEQ ID NO: 11 (nucleotides 37-57 and 311-330). In some embodiments, phase variations are determined in the promoter region of IR124 of Bacteroides fragilis NCTC 9343, that comprise IRs located at coordinates NC_003228.3:3420744_3420759_3421202_3421217, of the promoter as denoted by SEQ ID NO: 12 (nucleotides 17-32 and 475-490). In some embodiments, phase variations are determined in the promoter region of CPS1 of Bacteroides thetaiotaomicron VPI-5482, that comprise IRs located at coordinates NC_004663.1:463090_463119_463363_463392, of the promoter as denoted by SEQ ID NO: 13 (nucleotides 26-55 and 298-328). In some embodiments, phase variations are determined in the promoter region of CPS3 of Bacteroides thetaiotaomicron VPI-5482, that comprise IRs located at coordinates NC_004663.1:735864_735883_736132_736151, of the promoter as denoted by SEQ ID NO: 14 (nucleotides 20-39 and 287-307). In some embodiments, phase variations are determined in the promoter region of CPS5 of Bacteroides thetaiotaomicron VPI-5482, that comprise IRs located at coordinates NC_004663.1:2048014_2048032_2048274_2048292, of the promoter as denoted by SEQ ID NO: 15 (nucleotides 38-56 and 298-316). In some embodiments, phase variations are determined in the promoter region of CPS6 of Bacteroides thetaiotaomicron VPI-5482, that comprise IRs located at coordinates NC_004663.1:2124254_2124272_2124515_2124533, of the promoter as denoted by SEQ ID NO: 16 (nucleotides 28-46 and 289-307). In some embodiments, phase variations are determined in the promoter region of IR299 of Phocaeicola dorei MGYG-HGUT-02478, that comprise IRs located at coordinates NZ_LR699004.1:514152_514170_514322_514340, of the promoter as denoted by SEQ ID NO: 17 (nucleotides 45-63 and 215-233). In some embodiments, phase variations are determined in the promoter region of IR300 of Phocaeicola dorei MGYG-HGUT-02478, that comprise IRs located at coordinates NZ_LR699004.1:641746_641762_642003_642019, of the promoter as denoted by SEQ ID NO: 18 (nucleotides 33-49 and 289-306). In some embodiments, phase variations are determined in the promoter region of IR301 of Phocaeicola dorei MGYG-HGUT-02478, that comprise IRs located at coordinates NZ_LR699004.1:862176_862194_862369_862387, of the promoter as denoted by SEQ ID NO: 18 (nucleotides 33-49 and 289-306). In some embodiments, phase variations are determined in the promoter region of IR302 of Phocaeicola dorei MGYG-HGUT-02478, that comprise IRs located at coordinates NZ_LR699004.1:1538524_1538542_1538709_1538727, of the promoter as denoted by SEQ ID NO: 18 (nucleotides 33-49 and 289-306).

In some embodiments, phase variations or any product of phase variation (either direct nucleic acid-based product, e.g., mRNA, or protein product, or indirect product produced by the direct product) may be determined by the disclosed methods directly at the genomic loci, specifically at the genomic level. Optionally, using nucleic acid-based detecting molecules, or using methods that are based on nucleic acid molecule. In yet some further embodiments, phase variations in specific loci may be determined indirectly, by analyzing the direct or indirect encoded products of the genomic regions involved in the phase variations. Such direct or indirect encoded products may be determined and quantified using nucleic-acid-based detecting molecules (e.g., probes, primers, nucleic acid aptamers etc.), or alternatively or additionally, using amino-acid based detecting molecules (e.g., antibodies, enzymes, peptide aptamers or any other affinity molecule). Indirect gene products as used herein encompass any product produced directly or indirectly by a proteineous gene product encoded by the specific genomic regions. For example, if a genomic region undergoing phase variations encodes or regulates sequences encoding a protein product that participate in biosynthesis of carbohydrates, lipids, and the like. In such case, the phase variations may be determined using protein-based detecting molecules, for example, antibodies, or using enzymatic or chromatographic assays. Still further, in some embodiments, determination of phase variations and/or phase variation products by the methods of the preset disclosure may be performed at the genomic level and/or at nucleic acid products (e.g., mRNA) of the analyzed locus. In some embodiments, such determination may involve at least one of sequencing, amplification, hybridization, and molecular-weight based assays. More specifically, “Sequencing”, is the process of determining the nucleotide order of a given DNA or RNA fragment. There are various sequencing methods applicable in the present disclosure, to name but few, Sanger sequencing; capillary electrophoresis (CE) and fragment analysis; next generation sequencing (NGS) that can be grouped into two major categories: sequencing by hybridization (SBH) and sequencing by synthesis (SBS); whole-genome sequencing (WGS) and whole-exome sequencing (WES); whole-transcriptome sequencing; and targeted sequencing, which covers a relatively small set of genes or targeted regions of interest and may utilize Molecular Inversion Probes (MIPs). Additional sequencing methodologies applicable in the present disclosure, enable sequencing of long DNA (and RNA) molecules include, but are not limited to Pacific Biosciences (PacBio) sequencing, also referred to as SMRT (Singe Molecule Real Time) sequencing that enables very long fragments to be sequenced, up to 30-50 kb, or longer; and Nanopore sequencing. MinION, benchtop GridION, and a high throughput PromethION are examples of sequencers that may be utilized in the present methods. Still further, in some embodiments, the phase variation may be determined by the methods of the present disclosure using amplification methods. “Amplification” is a process by which a nucleic acid molecule is enzymatically copied to generate a progeny population with the same sequence as the parental one. The most widely used amplification method is Polymerase Chain Reaction (PCR). The result of a PCR amplification of a segment of DNA is called an “amplicon.” Nucleic acids can also be amplified in an isothermal reaction involving a reverse transcriptase, which copies RNA to DNA, and a DNA-dependent RNA polymerase, which transcribes DNA to RNA. Ligase-based methods, including the so-called Ligase Chain Reaction (LCR), can be also used for specific DNA or RNA amplification. Another general method for nucleic acid amplification involves cloning the selected DNA molecule into bacterial or eukaryotic cells, allowing them to reproduce, and collecting the amplified DNA. Other standard amplification methods are also applicable in the disclosed methods.

Still further, “Hybridization” or hybridization assay, may be applicable in the disclosed methods. More specifically, this is a type of Ligand Binding Assay (LBA) used to quantify nucleic acids in biological matrices. Hybridization assays can be in solution or on a solid support such as 96-well plates or labelled beads. Hybridization assays involve labelled nucleic acid probes to identify related DNA or RNA molecules (i.e. with significantly high degree of sequence similarity) within a complex mixture of unlabeled nucleic acid molecules. Another approach may be a “molecular weight based assay”, that refers to techniques used to separate compounds into their composite parts by their weight and/or structure properties. Molecular weight and/or structure properties can be measured using many different methods such as SDS-PAGE, blotting (Southern blot, Northern blot, Western blot), gel permeation chromatography (GPC), light scattering, ultracentrifugation and mass spectrometry. In yet some alternative and/or additional embodiments, the determination of phase variation or phase variation products may be performed at gene products of the analyzed locus. In some specific embodiments, the analysis may be performed using amino-acid based detecting molecules and assays, for example, at least one of affinity assay/s (immunological assay), molecular weight-based assay/s, scattering and mass spectrometry assay/s (MS).

Still further, “Immunological assays” rely on antigen-antibody interactions. Non limiting examples include Enzyme immunoassays (or EIAs), sometimes called enzyme-linked immunosorbent assays (or ELISA), immunoblot (e.g., Western blot), immunofluorescence assays, immunocytochemical staining, radioimmunoassay (RIA), optical immunoassay (OIA), lateral flow immunoassay, also called the immunochromatographic assay and time-resolved fluoroimmunoassay (TR-FIA). In some embodiments, scattering techniques may be applicable in the disclosed methods, and are a family of non-destructive analytical techniques which reveal information about the crystal structure, chemical composition, and physical properties of materials and thin films. Light scattering assay and X-ray are non-limiting examples of scattering techniques. “Mass spectrometry (MS)” that may be also applicable, is an analytical technique used to measure the mass-to-charge ratio of ions. It should be noted that the methods of the present disclosure may be applicable for any sample, specifically, any sample obtained from any subject or any sample of any analyzed media and/or habitat. In some embodiments, the sample may be any biological and/or environmental sample.

In some specific embodiments, the methods of the present disclosure may use any biological sample, for example, a biological sample as disclosed herein may comprise at least one of a body fluid and/or secretions and/or tissue sample. Still further, in some embodiments the samples may be obtained from the specific diagnosed subject or media and/or habitat or from any environment in the vicinity of the subject or media and/or habitat. For example, in case of a subject of the biological kingdom Plantae, a sample taken from the vicinity of the subject may be a sample obtained from the root area or the soil surrounding the roots.

The terms “sample”, “test sample” and “specimen” are used interchangeably in the present specification and claims and are used in its broadest sense. They are meant to include both biological and environmental samples and may include an exemplar of synthetic origin. In some embodiments herein, the biological sample is a fluid sample. Fluid sample include, but are not limited to, feces, saliva, mucosa, serum, urine, blood, plasma, cerebral spinal fluid (CSF), bronchoalveolar lavage (BAL) fluid, lymph, seminal plasma, pancreatic juice, breast milk, uterine, peritoneal cavity, lung lavage, or fluids collected from any organ or tissue cavity. For samples comprising tissues or organs, in some embodiments, the tissue may be a whole tissue, or selected parts of a tissue. Tissue parts can be isolated by micro-dissection of a tissue, or by biopsy, or by enrichment of sub-cellular compartments. Still further, biological samples including samples taken from various body regions (nose, throat, vagina, ear, eye, skin, sores), food products (both solids and fluids) and swabs taken from medicinal instruments, apparatus, materials), samples from various surfaces [hospitals, elderly homes, food manufacturing facilities, slaughter-houses pharmaceutical equipment (catheters etc), food preparation or packaging products), solutions and buffers], sewage etc. In some embodiments, the sample is at least one of a biological sample and an environmental sample. It should be noted that the term “sample” further refers to healthy as well as diseased or pathologically changed cells or tissues. Hence, the term further refers to a cell or a tissue associated with a disease, such a tumor, in particular cancer, and more specifically, melanoma, or alternatively inflamed colon tissue, or any lesion or wound. Alternatively, a sample of an injured organ or tissue of a subject, for example, liver tissue of a subject suffering from liver injury. In some embodiments, a sample can comprise cells that are placed in or adapted to tissue culture. A sample can additionally be a cell or tissue from any mammalian species, specifically, humans. A tissue sample can be further a fractionated or preselected sample, if desired, preselected or fractionated to contain or be enriched for particular cell types. In certain embodiments, the sample is obtained from a subject suffering from a disorder, or any biopsy of diseased tissue or organ. In some specific and non-limiting embodiments, the sample may be a feces sample. In yet some further embodiments, the sample analyzed by the disclosed methods may be a cell sample obtained from any tissue or organ of the examined subject. In some embodiments, the sample may be a skin cell sample. In yet some further embodiments, the sample may be any sample obtained by a biopsy from any tissue and/or organ. In some embodiments, the sample is a biopsy of a diseased tissue or organ. Still further in some embodiments, the sample may be a tumor biopsy.

“Biological samples” may be provided from animal, including human, fluid, solid (e.g., stool) or tissue, as well as liquid and solid food and feed products, food designed for human consumption, a sample including food designed for animal consumption, food matrices and ingredients such as dairy items, vegetables, meat and meat by-products, waste and sewage. In some embodiments, biological samples may include saliva, mucosa (nasal or oral swab samples), feces, serum, blood, urine, anterior nares (nasal swab) specimen collected by a healthcare professional or by onsite or home self-collection (using a flocked or spun polyester swab Nasopharyngeal (NP) specimens throat swab. Biological samples and specimens may be obtained from human as well as from all of the various families of domestic animals, as well as feral or wild animals, including, but not limited to, such animals as ungulates, bear, birds, fish, lagamorphs, rodents, etc.

As indicated herein before, the present disclosure may be also applicable for determining conditions in any media or habitat, and thus, a sample as used herein may further encompass any environmental sample. More specifically, “environmental samples” include environmental material such as surface matter, earth, soil, water, air and industrial samples, as well as samples obtained from food and dairy processing instruments, apparatus, equipment, utensils, disposable and non-disposable items. These examples are not to be construed as limiting the sample types applicable to the present disclosure. The sample may be any media, specifically, a liquid media that may contain the at least one microorganism that is analyzed for phase variations. The sample can be fractionated or preselected by a number of known fractionation or pre-selection techniques. A sample can also be any extract or filtrate of the above. The term also encompasses protein fractions or alternatively, nucleic acid from cells or tissue.

Still further, the methods of the preset disclosure provide diagnosis and determination of a specific physiological or environmental condition in any subject. In some embodiments, such subject may be at least one organism of the biological kingdom Animalia or of the biological kingdom Plantae. In some embodiments, the methods of the present disclosure may be applicable for any organism of the biological kingdom Animalia. In more specific embodiments, such organism may be any unicellular or multicellular invertebrate or vertebrate organism. More specifically, invertebrates, may be organisms of the Phylum Porifera—Sponges, the Phylum Cnidaria—Jellyfish, hydras, sea anemones, corals, the Phylum Ctenophora—Comb jellies, the Phylum Platyhelminthes—Flatworms, the Phylum Mollusca—Molluscs, the Phylum Arthropoda—Arthropods, the Phylum Annelida—Segmented worms like earthworm and the Phylum Echinodermata—Echinoderms. Still further, in some embodiments, the methods of the present disclosure may be applicable for any vertebrate organism. Vertebrates comprise all species of animals within the subphylum Vertebrata (chordates with backbones). The animals of the vertebrates group include Fish, Amphibians, Reptiles, Birds and Mammals (e.g., Marsupials, Primates, Rodents and Cetaceans). Vertebrates include the jawless fish and the jawed vertebrates, which include the cartilaginous fish (sharks, rays, and ratfish) and the bony fish. In more specific embodiments, the subject of the present disclosure may be a mammal, for example, any member of the mammalian nineteen orders, specifically, Order Artiodactyla (even-toed hoofed animals), Order Carnivora (meat-eaters), Order Cetacea (whales and porpoises), Order Chiroptera (bats), Order Dermoptera (colugos or flying lemurs), Order Edentata (toothless mammals), Order Hyracoidae (hyraxes, dassies), Order Insectivora (insect-eaters), Order Lagomorpha (pikas, hares, and rabbits), Order Marsupialia (pouched animals), Order Monotremata (egg-laying mammals), Order Perissodactyla (odd-toed hoofed animals), Order Pholidata, Order Pinnipedia (seals and walruses), Order Primates (primates), Order Proboscidea (elephants), Order Rodentia (gnawing mammals), Order Sirenia (dugongs and manatees), Order Tubulidentata (aardvarks). In yet some further embodiments, the present disclosure may be applicable for any organism of the order primates. In yet some further embodiments, the present disclosure may be applicable for any organism of the subfamily Homininae, that includes the hylobatidae (gibbons) and the hominidae that includes ponqunae (orangutans) and homininae [gorillini (gorilla) and hominini ((panina (chimpanzees) and hominina (humans))]. Thus, in some embodiments, a subject as disclosed herein relates to a human subject. In some embodiments, the human subject may be of any sex, ethnic group, age or physical or mental condition. In some specific embodiment, the methods of the present disclosure may be applicable for a mammal that may be at least one of a Cattle, domestic pig (swine, hog), sheep, horse, goat, alpaca, lama and Camels. More specifically, the subject the present disclosure as well as the methods disclosed herein above offer great economic advantage for any industrial or agricultural use of animals, specifically, livestock. Thus, in some specific embodiments, the present disclosure may be applicable for mammalian livestock, specifically those used for meat, milk, eggs, fur, leather, and wool industries. In yet some further embodiments, the organism applicable in the methods of the present disclosure, may be avian organisms. In yet some further specific embodiments, the present disclosure may be suitable for birds. More specifically, domesticated and undomesticated birds are also suitable organisms for the present disclosure.

Still further, in some embodiments the organism of the biological kingdom Plantae may be a dioecious plant, specifically, a plant presenting biparental reproduction. In some specific embodiments, the plant may be of the family Cannabaceae, specifically, any one of Cannabis (hemp, marijuana) and Humulus (hops). In more specific embodiments, the plant of the family Cannabaceae may be Cannabis (hemp, marijuana). In yet some further embodiments, the plant of the family Cannabaceae may be Humulus (hops).

In some embodiments, any plants are applicable in the present disclosure, for example, any model plants such as, Arabidopsis, Tobacco, Solanum licopersicum, Solanum tuberosum. In yet some further embodiments, Canola, Cereals (Corn wheat, Barley), rice, sugarcane, Beet, Cotton, Banana, Cassava, sweet potato, lentils, chickpea, peas, Soy, nuts, peanuts, Lemna, Apple, may be applicable in the present disclosure.

In some embodiments, the subject diagnosed by the disclosed methods may be any subject of the biological kingdom Animalia. In more specific embodiments, such subject may be at least one mammalian subject. Still further, in some embodiments, the methods of the present disclosure may be applicable to a human subject. In more specific embodiments, such human subject may be of any gender, any ethnic group, at any age, and any healthy or diseased condition. Still further, in some embodiments, the methods of the present disclosure may be useful for detecting any physiological or environmental state. In some embodiments, such environmental state of a subject and or a media and/or habitat may comprise in some embodiments at least one of: exposure to at least one biotic and/or at least one abiotic stimulus, various pH conditions, salinity, oxidative stress, temperature, humidity, draught and the like. In some embodiments, media and/or habitat comprises solid and/or liquid media and/or habitat (earth, soil, water, industrial samples, samples obtained from food and dairy processing (Yogurt), Petroleum. In some specific embodiments, the methods of the present disclosure may be applicable for detecting a pathological condition in a subject. According to such embodiments, the method comprising the following steps. One step (a), involves determining phase variation in at least one locus of at least one microorganism in at least one sample of the subject, to obtain at least one phase variation value of the sample for at least one of the loci in the at least one microorganism. In some embodiments, the next step may involve determining if the at least one phase variation value obtained for the sample in step (a), is positive or negative with respect to a reference phase variation value or with respect to a phase variation value determined for at least one control sample. Another step (b), concerns classifying the subject as affected by, and/or suffering from said pathologic disorder, if the at least one phase variation value obtained for the sample in step (a), is positive with respect to a reference phase variation value (standard or cutoff value) pre-determined for the pathologic disorder, or with respect to a phase variation value determined for at least one control sample, thereby detecting the pathological disorder in the subject. It should be appreciated that any of the disclosed methods of pathological disorders as specified herein, may further comprise the step of identifying at least one locus displaying phase variation that characterizes the at least one diagnosed pathological disorder or condition. Specifically, the methods as disclosed herein above. In some specific embodiments, the diagnostic methods of the present disclosure may be applicable for detecting at least one immune-related disorder in a subject. In yet some particular embodiments, such methods may comprise the following steps:

In one step (a), determining phase variation in at least one of the PSA, CPS, helix-turn-helix transcriptional regulator or DUF6198 family protein, HAD family phosphatase or MATE family efflux transporter, ATP-binding protein, UpxY family transcription antiterminator, TonB-dependent receptor loci of at least one bacterium in at least one sample of said subject, to obtain at least one phase variation value of the sample for at least one of the loci in at least one bacterium. The method may further comprise the step of determining if the at least one phase variation value obtained for the sample in step (a), is positive or negative with respect to a reference phase variation value or with respect to a phase variation value determined for at least one control sample. Another step (b), involves classifying the subject as affected by, and/or suffering from the pathologic disorder, if the at least one phase variation value obtained for said sample in step (a), is positive with respect to a reference phase variation value (or cutoff value) pre-determined for the immune-related disorder, or with respect to a phase variation value determined for at least one control sample, thereby detecting the immune-related disorder in the subject.

In more specific embodiments, the immune-related disorder may be an inflammatory disorder. In yet some further specific embodiments, such inflammatory disorder may be IBD. Thus, the disclosed methods may be applicable for the diagnosis of IBD. The disclosed method comprising the following steps. In one step (a), determining phase variation in at least one of the PSA and the CPS loci of at least one Bacteroidota bacterium in at least one sample of the subject, to obtain at least one phase variation value of the sample. In some embodiments, the method father involves determining if the at least one phase variation value obtained for the sample in step (a), is positive or negative with respect to a reference phase variation value or with respect to a phase variation value determined for at least one control sample. Another step (b), involves classifying the subject as affected by, and/or suffering from IBD, if the at least one phase variation value obtained for the sample in step (a), is positive with respect to a reference phase variation value (or cutoff value) pre-determined for IBD patients, or with respect to a phase variation value determined for at least one control sample, thereby detecting IBD in the subject.

In some embodiments, phase variations comprise at least one inversion in at least one promoter region of at least one gene residing with said PSA and/or CPS loci, thereby converting the ON/OFF orientation of the at least one promoter region/s. In some embodiments, an inverted orientation is indicated herein as the OFF orientation of the at least one promoter in any of the indicated loci. In some embodiments, an inverted orientation referred to herein as “OFF” orientation leads to shut down of the expression of at least one gene regulated by the inverted promoter region. In yet some further embodiments, “OFF” orientation leads to the expression of an alternative gene product, e.g., encoded by the opposite orientation with respect to the at least one gene regulated by the same promoter region at the “ON” orientation. In some embodiments, a phase variation value reflects the ratio between inverted and non-inverted orientation at a specific locus as determined for specific bacteria in a specific sample. In some embodiments, an increased inversion may be reflected by a phase variation value that is greater than 1, specifically, more than 50% of the bacteria in the bacterial population of the sample, display the inverted orientation, e.g., the OFF orientation. In some specific embodiments, the Bacteroides fragilis population in the sample display an inverted orientation (specifically, “OFF” orientation) of at least one promoter of the PSA locus. Thus, in some embodiments, a phase variation value that is greater than 1, determined for at least one promoter of the PSA locus in Bacteroides fragilis of a sample of a subject, indicates that the subject is suffering from IBD, specifically, UC and/or CD. In yet some further specific embodiments, a phase variation value that is smaller than 1, determined for at least one promoter of the CPS locus in Bacteroides thetaiotaomicron and/or Phocaeicola dorei, of a sample of a subject, indicates that the subject is suffering from IBD, specifically, UC and/or CD. In yet some further embodiments, the immune-related disorder may be a proliferative disorder. In more specific embodiments, such proliferative disorder may be a malignant neoplastic disorder. Thus, the methods of the present disclosure may be applicable for diagnosing cancer, specifically, melanoma. In some embodiments, the disclosed methods may comprise in one step (a), determining phase variation in at least one of the helix-turn-helix transcriptional regulator or DUF6198 family protein, HAD family phosphatase or MATE family efflux transporter, ATP-binding protein, UpxY family transcription antiterminator, TonB-dependent receptor loci of at least one Bacteroidota bacterium in at least one sample of the subject, to obtain at least one phase variation value of the sample.

Still further, in some optional embodiments the method further comprises determining if the at least one phase variation value obtained for the sample in step (a), is positive or negative with respect to a reference phase variation value or with respect to a phase variation value determined for at least one control sample. In another step (b) of the disclosed method, the subject is classified a subject as affected by, and/or suffering from melanoma, if the at least one phase variation value obtained for the sample in step (a), is positive with respect to a reference phase variation value (or cutoff value) pre-determined for melanoma patients, or with respect to a phase variation value determined for at least one control sample, thereby detecting melanoma in the subject.

A further aspect of the preset disclosure relates to a prognostic method for predicting and assessing responsiveness of a subject suffering from a pathologic disorder, to at least one therapeutic agent or a treatment regimen comprising the at least one therapeutic agent, and optionally for monitoring disease progression. In more specific embodiments, the disclosed methods comprise the steps of: in one step (a), determining phase variation in at least one locus of at least one microorganism in at least one sample of the subject, to obtain at least one phase variation value of the sample for at least one of the loci in the at least one microorganism. In some embodiments, the method may further comprise determining if the at least one phase variation value obtained for the sample in step (a), is positive or negative with respect to a reference phase variation value or with respect to a phase variation value determined for at least one control sample. Another step (b) involves classifying the subject as: (i) a responder subject to the at least one therapeutic agent or treatment regimen, if the at least one phase variation value obtained in step (a) for at least one sample obtained after the initiation of the treatment regimen and/or a sample of the subject contacted with the therapeutic agent, is negative with respect to a reference phase variation value (or cutoff value) pre-determined for the pathologic disorder, or with respect to a phase variation value determined for at least one control sample; or (ii) a non-responder subject to the at least one therapeutic agent or treatment regimen, if the at least one phase variation value obtained in step (a) for at least one sample obtained after the initiation of the treatment regimen and/or a sample of the subject contacted with the therapeutic agent, is positive with respect to a reference phase variation value (or cutoff value) pre-determined for the pathologic disorder, or with respect to a phase variation value determined for at least one control sample. Thereby, predicting and assessing responsiveness of the subject to the treatment regimen.

In some embodiments of the prognostic method of the present disclosure, the step of monitoring disease progression may comprise at least one of predicting and determining disease relapse and assessing a remission interval. In some embodiments, such methods further comprise the steps of: in step (c), repeating step (a) to determine at least one phase variation value for at least one of the loci of the at least one bacterium, in at least one more temporally-separated sample of the subject. Step (d), further involves predicting and/or determining disease relapse in the subject, if at least one temporally separated sample obtained after the initiation of the treatment regimen displays at least one phase variation value that is positive with respect to a reference phase variation value (or cutoff value) pre-determined for the pathologic disorder, or with respect to a phase variation value determined for at least one control sample.

It should be understood that in some embodiments, the prognostic methods of the preset disclosure may further comprise an additional step of identifying at least one locus displaying phase variation, that characterizes responsive or non-responsive subjects to the treatment regimen. It should be noted that “Prognostic”, is defined as a forecast of the future course of a disease or disorder, based on medical knowledge. The term “responder” or “responsiveness” to a certain treatment, refers to an improvement in at least one relevant clinical parameter as compared to an untreated subject diagnosed with the same pathology (e.g., the same type, stage, degree and/or classification of the pathology), or as compared to the clinical parameters of the same subject prior to treatment with said medicament. The term “non responder” or “non-responsiveness” or “drug resistance” to treatment with a specific medicament, refers to a patient not experiencing an improvement in at least one of the clinical parameter and is diagnosed with the same condition as an untreated subject diagnosed with the same pathology (e.g., the same type, stage, degree and/or classification of the pathology), or experiencing the clinical parameters of the same subject prior to treatment with the specific medicament. In yet some further embodiments the subject may be further sub classified with respect to the expected degree, depth or extent and/or duration of responsiveness, for example as a poor responder, a responder displaying mild response, a responder displaying a good response or even a responder displaying excellent response, and the like. Loss of responsiveness refers to a situation wherein a responder experiences a decline in at least one of the clinical parameters that showed an improvement in a past examination.

The disclosed method may further be used for predicting the disease condition, for example, predict or revel relapse of a disease. The term “relapse”, as used herein, relates to the re-occurrence of a condition, disease or disorder that affected a person in the past. Specifically, the term relates to the re-occurrence of a disease being treated a regimen.

Still further, the method may be used for assessing a remission interval. “Remission” or “remission interval” as used herein is either the reduction or disappearance of the signs and symptoms of a disease. The term may also be used to refer to the period during which this reduction occurs. A remission may be considered a partial remission or a complete remission. Each disease, type of disorder, or clinical trial can have its own definition of a partial remission. As indicated herein, the prognostic methods may use various samples obtained from different time-points, for monitoring a subject. The samples are therefore referred to herein as temporally separated samples. In some embodiments, the at least one more temporally-separated sample may be obtained after the initiation of at least one treatment regimen. It should be understood that in some particular embodiments, at least one sample may be obtained prior to initiation of the treatment. However, in some embodiments, the methods disclosed herein may be applied to subjects already treated by a treatment regimen. Such monitoring may therefore provide a powerful therapeutic tool used for improving and personalizing the treatment regimen offered to the treated subject.

In some embodiments, at least two “temporally-separated” test samples must be collected from the examined patient and compared thereafter, in order to determine if there is any change or difference in the phase variation value between the samples. Such change may reflect a change in the responsiveness of the subject. In practice, to detect a change having more accurate predictive value, at least two “temporally-separated” test samples and preferably more, must be collected from the patient. The number of samples collected and used for evaluation and classification of the subject either as a responder or alternatively, a non-responder or as a subject that may experience relapse of the disease, may change according to the frequency with which they are collected. For example, the samples may be collected at least every day, every two days, every four days, every week, every two weeks, every three weeks, every month, every two months, every three months every four months, every 5 months, every 6 months, every 7 months, every 8 months, every 9 months, every 10 months, every 11 months, every year or even more. Furthermore, to assess the disease progression according to the present disclosure, it is understood that the change in phase variation value, may be calculated as an average change over at least three samples taken in different time points, or the change may be calculated for every two samples collected at adjacent time points. It should be appreciated that the sample may be obtained from the monitored patient in the indicated time intervals for a period of several months or several years. More specifically, for a period of 1 year to a period of 15 years or more.

In some embodiments, the prognostic methods of the present disclosure may analyze any microorganism in a sample of the subject. In some embodiments, such at least one microorganism may be at least one bacterium. Still further, in some embodiments, the bacteria analyzed for phase variations by the disclosed methods may comprise at least one bacterium of at least one phylum selected from Bacteroidota, Verrucomicrobiota, proteobacteria, actinobacteria, firmicutes and Tenericutes. In yet some further embodiments, the bacteria analyzed by the prognostic methods of the present disclosure may comprise at least one of Bacteroides fragilis, Bacteroides thetaiotaomicron, Phocaeicola dorei, Bacteroides cellulosilyticus, Bacteroides ovatus, Bacteroides stercorirosoris, or any isolate or species thereof.

Still further, in some embodiments, the prognostic methos of the present disclosure determine phase variation in at least one locus of at least one bacterium in at least one sample of the prognosed subject. The phase variations determined comprise phase variation/s in at least one intergenic region/s and/or intragenic region/s of the analyzed loci. Still further, in some embodiments, the prognosed subject is a subject suffering from any pathologic disorder. In some embodiments, the pathologic disorder may be at least one immune-related disorder. According to some embodiments, in case of immune-related disorders, the analyzed loci for determining phase variation may be determined in at least one of the PSA, CPS, helix-turn-helix transcriptional regulator or DUF6198 family protein, HAD family phosphatase or MATE family efflux transporter, ATP-binding protein, UpxY family transcription antiterminator, TonB-dependent receptor loci of at least one Bacteroidota bacterium in at least one sample of the subject.

In some embodiments, the immune-related disorder is any inflammatory disorder. In more specific embodiments, the disclosed prognostic methods may be applicable for inflammatory disorders such as IBD. For IBD patients, the disclosed prognostic method may be applicable for determining responsiveness to any therapeutic agent, such therapeutic agent may comprise an agent directed against an inflammatory cytokine. An “inflammatory cytokine” or proinflammatory cytokine as used herein include interleukin-1 (IL-1), IL-6, IL-12, and IL-18, tumor necrosis factor alpha (TNF-α), interferon gamma (IFNγ), and granulocyte-macrophage colony stimulating factor (GM-CSF). In some specific and nonlimiting embodiments, the prognostic methods may be applicable for determining the responsiveness of a subject suffering from IBD, to a treatment regimen directed against TNF-α, for example, at least one anti TNF-α antibody (e.g., Infliximab, IFX or Humira)). In some embodiments, the disclosed prognostic methods comprise: in one step (a), determining phase variation in at least one of the PSA and the CPS loci of at least one Bacteroidota bacterium in at least one sample of the subject, to obtain at least one phase variation value of the sample. In some embodiments, the disclosed method comprises determining if the at least one phase variation value obtained for the sample in step (a), is positive or negative with respect to a reference phase variation value or with respect to a phase variation value determined for at least one control sample. Another step (b), involves classifying the subject. More specifically, in some embodiments, the subject is classified as (i), a responder subject to the therapeutic agent directed against an inflammatory cytokine (e.g., IFX), if the at least one phase variation value obtained in step (a) for at least one sample obtained after the initiation of the treatment regimen and/or a sample of the subject contacted with said agent, is negative with respect to a reference phase variation value (or cutoff value) pre-determined for IBD, and/or for non-responders, or with respect to a phase variation value determined for at least one control sample. The subject is classified as (ii), a non-responder subject to the at least one agent directed against an inflammatory cytokine (e.g., IFX), if the at least one phase variation value obtained in step (a) for at least one sample obtained after the initiation of the treatment regimen and/or a sample of the subject contacted with the agent, is positive with respect to a reference phase variation value (or cutoff value) pre-determined for said IBD, or with respect to a phase variation value determined for at least one control sample. In some alternative embodiments, the immune-related disorder may be a proliferative disorder, specifically, a malignant neoplasm. In some specific embodiments, such prognostic method may be applicable for patients suffering from melanoma. In yet some further embodiments, the disclosed prognostic methods may be applicable for evaluating the responsiveness of melanoma patients to a treatment regimen comprising at least one immune checkpoint inhibitor compound. “Immune checkpoint inhibitors” are immunotherapy drugs regulating (stimulating or inhibiting) the immune system, and/or an immune response. Stimulatory checkpoint molecules are members of the tumor necrosis factor (TNF) receptor superfamily, CD27, CD40, OX40, GITR and CD137, while inhibitory include CTLA-4, PD1, PDL-1, B7-H3 (CD276), B7-H4 (VTCN1), BTLA, (CD272), IDO, KIR, LAG3, NOX2, TIM-3, VISTA, (SIGLEC9). Cancer therapy involves the use of compounds that inhibit the action of the inhibitory immune-checkpoint molecules, specifically, any of those indicated above. In some embodiments, the therapeutic compound may be an inhibitor of PD-1L. For example, at least one anti-PD-1L antibody. In some embodiments, the disclosed prognostic methods comprise: In one step (a), determining phase variation in at least one of the helix-turn-helix transcriptional regulator or DUF6198 family protein, HAD family phosphatase or MATE family efflux transporter, ATP-binding protein, UpxY family transcription antiterminator, TonB-dependent receptor loci of at least one Bacteroidota bacterium in at least one sample of the subject, to obtain at least one phase variation value of the sample.

In some embodiments, the method further comprises determining if the at least one phase variation value obtained for the sample in step (a), is positive or negative with respect to a reference phase variation value or with respect to a phase variation value determined for at least one control sample. In another step (b), classifying the subject. More specifically, the subject is classified as (i), a responder subject to the at least one immune checkpoint inhibitor compound (e.g., anti-PD-1L), if the at least one phase variation value obtained in step (a) for at least one sample obtained after the initiation of the treatment regimen and/or a sample of the subject contacted with the agent, is negative with respect to a reference phase variation value (or cutoff value) pre-determined for the melanoma and/or non-responsive subjects, or with respect to a phase variation value determined for at least one control sample. The subject is classified as (ii), a non-responder subject to the at least one immune checkpoint inhibitor compound (e.g., anti-PD-1L), if the at least one phase variation value obtained in step (a) for at least one sample obtained after the initiation of the treatment regimen and/or a sample of the subject contacted with the at least one immune checkpoint inhibitor compound (e.g., anti-PD-1L), is positive with respect to a reference phase variation value (or cutoff value) pre-determined for melanoma and/or non-responders, or with respect to a phase variation value determined for at least one control sample. In some embodiments, phase variation or any phase variation direct or indirect products, may be analyzed by the disclosed prognostic methods and determined at the genomic level and/or directly or indirectly at the gene-product level. In some optional embodiments, determination of phase variations is performed using nucleic acid-based and/or amino-acid-based detecting molecules. In some embodiments, determination of phase variation or phase variation product/s at the nucleic acid level may comprise at least one of sequencing, amplification, hybridization, and molecular-weight based assays. In some embodiments, determination of phase variation or phase variation product/s at the protein level comprises at least one of affinity assay/s (immunological assay), molecular weight-based assay/s, scattering and mass spectrometry assay/s (MS).

A further aspect of the present disclosure relates to a method for determining a personalized treatment regimen for a subject suffering from a pathologic disorder. In some embodiments, the personalized therapeutic methods disclosed herein may comprise the following steps. In one step (I), assessing responsiveness of a subject suffering from a pathologic disorder to at least one therapeutic agent or a treatment regimen comprising the at least one therapeutic agent. In some embodiments, such assessment may be performed by the steps of: first (a), determining phase variation in at least one locus of at least one microorganism in at least one sample of the subject, to obtain at least one phase variation value of the sample for at least one of the loci in the at least one microorganism (e.g., bacterium). In some embodiments, the method further comprises determining if the at least one phase variation value obtained for the sample in step (a), is positive or negative with respect to a reference phase variation value (e.g., a predetermined standard, or cutoff value) or with respect to a phase variation value determined for at least one control sample. In step (b), the subject is classified as a responder or a non-responder subject.

More specifically, (i) the subject is classified as a responder to the at least one therapeutic agent or treatment regimen, if the at least one phase variation value obtained in step (a) for at least one sample obtained after the initiation of the treatment regimen and/or a sample of the subject contacted with the therapeutic agent, is negative with respect to a reference phase variation value (or cutoff value) pre-determined for the pathologic disorder, or with respect to a phase variation value determined for at least one control sample. Alternatively (ii), the subject is classified as a non-responder to the at least one therapeutic agent or treatment regimen, if the at least one phase variation value obtained in step (a) for at least one sample obtained after the initiation of the treatment regimen and/or a sample of the subject contacted with the therapeutic agent, is positive with respect to a reference phase variation value (or cutoff value) pre-determined for the pathologic disorder, or with respect to a phase variation value determined for at least one control sample. In step (II), after classification of the subject, a treatment regimen is selected based on the determined responsiveness. More specifically, in some embodiments, selecting a treatment regimen based on the responsiveness comprises one of the following options: One option (i), administering the therapeutic agent to a subject classified as a responder, and/or maintaining the treatment regimen for a subject classified as a responder.

Another option (ii), concerns ceasing a treatment regimen comprising the at least one therapeutic agent for a subject displaying disease relapse and/or loss of responsiveness, non-responsiveness, poor-responsiveness and/or drug-resistance. It should be thus appreciated that in some embodiments, the disclosed methods may further comprise the step of administration to the subject, and may thus provide personalized therapeutic methods. In some embodiments, assessing responsiveness of a subject suffering from a pathologic disorder to at least one therapeutic agent or a treatment regimen comprising the at least one therapeutic agent is performed by the method as defined by the present disclosure.

As mentioned above, disclosed herein is a method for determining a personalized treatment regimen for a subject suffering from a pathologic disorder. A “Personal treatment”, as used herein, refers to treatment which is tailored to the individual patient based on their predicted response or risk of disease. This term further encompasses any future monitoring, prediction and management of relapse and chances for response during relapse. “Treatment regimen” as used herein refers to the course of treatment type including the drugs to be used, their dosage, the frequency and duration of treatments, and other considerations defined based on medical decisions. Therefore, in some embodiments, a treatment regimen for a subject displaying a disease relapse and/or loss of responsiveness, non-responsiveness, poor-responsiveness and/or drug-resistance may be ceased (i.e. stopped) and optionally replaced by an alternative treatment regimen.

A further aspect of the present disclosure relates to a method for modulating a physiological and/or environmental state and/or condition in a subject in need thereof, and/or in a media and/or habitat. More specifically, the method comprising: In step (I), determining a physiological and/or environmental condition or state of a subject or a media and/or habitat, by the following steps. Step (a) of determining the physiological and/or environmental condition or state of a subject or a media and/or habitat, involves determining phase variation in at least one locus of at least one microorganism in at least one sample of the subject or media and/or habitat, to obtain at least one phase variation value of the sample. The method may further comprise determining if the at least one phase variation value obtained for the sample in step (a), is positive or negative with respect to a reference phase variation value (e.g., standard value and/or cutoff value) pre-determined for the physiological or environmental condition or state or with respect to a phase variation value determined for at least one control sample. It should be understood that in some embodiments, determination of the phase variation value is pre-determined for microorganisms collected from two or more groups of subjects or media/habitats characterized by the specific physiological and/or environmental condition. Step (b), involves classifying the subject and/or media and/or habitat as displaying the physiological and/or environmental condition or state, if the at least one phase variation value obtained for the sample in step (a), is positive with respect to a reference phase variation value (or cutoff value) pre-determined for the physiological and/or environmental condition or state, or with respect to a phase variation value determined for at least one control sample. The physiological or environmental state is thereby determined for a subject or a media and/or habitat. The next step (II), of the modulatory methods of the present disclosure concerns subjecting the subject and/or media and/or habitat classified as displaying the physiological and/or environmental condition or state to at least one physiological condition and/or compound that modulates the physiological condition and/or state, thereby modulating the physiological and/or environmental state and/or condition.

In some embodiments, the step of determining a physiological and/or environmental condition or state of a subject or a media and/or habitat of (I), is performed by any of the methods defined herein above in the present disclosure. The term “modulating” as used herein encompasses any change or modification in the physiological and/or environmental state and/or condition in a subject in need thereof, and/or a media and/or habitat. Such change may include an increase, enhancement or decrease in at least one of physiological functioning and/or the environmental conditions in relation to a control or a normal or a baseline level of the physiological functioning and/or the environmental conditions determined under certain condition”.

A further aspect of the present disclosure relates to a method for treating, preventing, inhibiting, reducing, eliminating, protecting or delaying the onset of at least one pathologic disorder in a subject. More specifically, in some embodiments the method comprising the following steps. Step (I) involves detecting a pathological condition in a subject by the steps of: in step (a) determining phase variation in at least one locus of at least one microorganism (e.g., bacteria) in at least one sample of the subject, to obtain at least one phase variation value of the sample. In some embodiments, detection of the pathological disorder in the subject may further involve determining if the at least one phase variation value obtained for the sample in step (a), is positive or negative with respect to a reference phase variation value or with respect to a phase variation value determined for at least one control sample. Step (b) of the detection of the pathological disorder in the subject involves classifying the subject as affected by, and/or suffering from the pathologic disorder, if the at least one phase variation value obtained for the sample in step (a), is positive with respect to a reference phase variation value (or cutoff value) pre-determined for the pathologic disorder, or with respect to a phase variation value determined for at least one control sample, thereby detecting said pathological disorder in the subject. In some embodiments, where the value determined is negative, the subject is not suffering from the disorder. It should be understood that the diagnostic step of the disclosed therapeutic method may further comprise the preliminary step of identifying the specific loci that display phase variations that characterize the specific pathologic disorder, as specified above in connection with previous aspects of the present disclosure. The diagnostic step of the disclosed therapeutic method is followed by step (II) that involves administering to a subject classified as affected by, and/or suffering from the disorder a therapeutically effective amount of at least one therapeutic compound.

A further aspect of the present disclosure relates to at least one therapeutic compound for use in a method for treating, preventing, inhibiting, reducing, eliminating, protecting or delaying the onset at least one pathological disorder in a subject, wherein the therapeutic method further comprises a diagnostic step. Specifically, detecting the pathological condition in a subject by: (a), determining phase variation in at least one locus of at least one microorganism (e.g., bacteria) in at least one sample of the subject, and (b), classifying the subject as discussed herein above. The therapeutic methods disclosed herein provide tailored and monitored treatment as discussed above, by combining a diagnostic step that allows determination of the specific state of the subject and evaluation of the effect of a particular therapeutic compound on each treated subject.

It is to be understood that the terms “treat”, “treating”, “treatment” or forms thereof, as used herein, mean preventing, ameliorating or delaying the onset of one or more clinical indications of disease activity in a subject having a pathologic disorder. Treatment refers to therapeutic treatment. Those in need of treatment are subjects suffering from a pathologic disorder. Specifically, providing a “preventive treatment” (to prevent) or a “prophylactic treatment” is acting in a protective manner, to defend against or prevent something, especially a condition or disease. The term “treatment or prevention” as used herein, refers to the complete range of therapeutically positive effects of administrating to a subject including inhibition, reduction of, alleviation of, and relief from, an immune-related condition and illness, immune-related symptoms or undesired side effects or immune-related disorders. More specifically, treatment or prevention of relapse or recurrence of the disease, includes the prevention or postponement of development of the disease, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms. It should be appreciated that the terms “inhibition”, “moderation”, “reduction”, “decrease” or “attenuation” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more. With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively. The term “amelioration” as referred to herein, relates to a decrease in the symptoms, and improvement in a subject's condition brought about by the methods according to the present disclosure, wherein said improvement may be manifested in the forms of inhibition of pathologic processes associated with the immune-related disorders described herein, a significant reduction in their magnitude, or an improvement in a diseased subject physiological state. The term “inhibit” and all variations of this term is intended to encompass the restriction or prohibition of the progress and exacerbation of pathologic symptoms or a pathologic process progress, said pathologic process symptoms or process are associated with.

The term “eliminate” relates to the substantial eradication or removal of the pathologic symptoms and possibly pathologic etiology, optionally, according to the methods of the present disclosure described herein. The terms “delay”, “delaying the onset”, “retard” and all variations thereof are intended to encompass the slowing of the progress and/or exacerbation of a disorder associated with the immune-related disorders and their symptoms slowing their progress, further exacerbation or development, so as to appear later than in the absence of the treatment according to the present disclosure. It should be noted that the terms “disease”, “disorder”, “condition” and “illness”, are equally used herein. It should be appreciated that any of the methods described by the present disclosure may be applicable for treating and/or ameliorating any of the disorders disclosed herein or any condition associated therewith. It is understood that the interchangeably used terms “associated”, “linked” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology. More specifically, as used herein, “disease”, “disorder”, “condition”, “pathology” and the like, as they relate to a subject's health, are used interchangeably and have meanings ascribed to each and all of such terms.

A further aspect of the present disclosure relates to a screening method for identifying and/or evaluating at least one therapeutic compound for the treatment of a pathologic disorder. In some embodiments, the method comprising the following steps. In step (a), determining phase variation in at least one locus of at least one microorganism in at least one sample contacted with a candidate compound, to obtain at least one phase variation value of the sample for at least one of the loci in the at least one microorganism (e.g., bacteria). In some embodiments, the sample is of a subject suffering from the pathologic disorder. In the next step (b), determining that the candidate compound is a therapeutic compound for the disorder if the at least one phase variation value obtained for the sample in step (a), is negative with respect to a reference phase variation value (or cutoff value) pre-determined for the pathologic disorder, or with respect to a phase variation value determined for at least one control sample not contacted with the candidate compound.

A “candidate compound” refers herein to a therapeutic compound which meets certain efficacy criteria for clinical use or for further drug development. The candidate compound may be any inorganic or organic molecule, any small molecule, nucleic acid-based molecule, any aptamer, any peptide (L- as well as D-aa residues), any lipid, any carbohydrate or any combinations thereof. The candidate may be any natural or synthetic molecule. The candidate may be any chimeric or fusion protein, or any small molecule-peptide conjugate, or any of the compounds disclosed by the present disclosure. A compound to be tested may be referred to as a test compound or a candidate compound. Any compound may be used as a test or a candidate compound in various embodiments. In some embodiments a library of FDA approved compounds appropriate for human may be used. Compound libraries are commercially available from any known company. Still further, libraries of natural compounds in the form of bacterial, fungal, plant and animal extracts are commercially available or can be readily prepared by methods well known in the art. Compounds isolated from natural sources, such as animals, bacteria, fungi, plant sources, and marine samples may be tested for the presence of potentially useful pharmaceutical compounds. It will be understood that the agents to be screened could also be derived or synthesized from chemical compositions or man-made compounds. In certain embodiments, the candidate compound may be any compound, for example, at least one of a small molecule, aptamer, a peptide, a nucleic acid molecule and an immunological agent, and any combinations thereof. A “small molecule” as used herein, is an organic molecule that is less than about 2 kilodaltons (kDa) in mass. “Aptamers” are short sequences of artificial DNA, RNA, XNA, or peptide that bind a specific target molecule, or family of target molecules. They exhibit a range of affinities (KD in the pM to μM range), and are sometimes classified as “chemical antibodies” or “antibody mimics”. The nucleic acid-based structure of aptamers, which are mostly oligonucleotides, is very different from the amino acid-based structure of antibodies, which are proteins. Aptamers are usually obtained by selection from a large random sequence library, using methods well known in the art, such as SELEX and/or Molinex.

The term “peptide” or “polypeptide” as used herein refers to amino acid residues, connected by peptide bonds. A peptide sequence is generally reported from the N-terminal end containing free amino group to the C-terminal end containing free carboxyl group and may include any chain or polymeric chain of amino acids. In some embodiments, a peptide has an amino acid sequence that occurs in nature. In some embodiments, a peptide has an amino acid sequence that does not occur in nature. In some embodiments, a peptide has an amino acid sequence that contains portions that occur in nature separately from one another (i.e., from two or more different organisms, for example, human and non-human portions). In some embodiments, a peptide has an amino acid sequence that is engineered in that it is designed and/or produced through action of the hand of man. The term “nucleic acid”, “nucleic acid molecule”, or “polynucleotide” refers to polymers of nucleotides, and includes but is not limited to deoxyribonucleic acid (DNA), ribonucleic acid (RNA), DNA/RNA hybrids including polynucleotide chains of regularly and/or irregularly alternating deoxyribosyl moieties and ribosyl moieties (i.e., wherein alternate nucleotide units have an —OH, then and —H, then an —OH, then an —H, and so on at the 2′ position of a sugar moiety), and modifications of these kinds of polynucleotides, wherein the attachment of various entities or moieties to the nucleotide units at any position are included.

Still further, the candidate may comprise in some embodiments, any immunologic agents. “Immunologic agents” refer herein to drugs that can modify the immune response, either by enhancing or suppressing the immune system. Non limiting examples include immunoglobulins, immunostimulants, (such as bacterial vaccines, colony stimulating factors, interferons, interleukins, other immunostimulants, therapeutic vaccines, vaccine combinations and viral vaccines), immunosuppressive agents. It should be appreciated that the various candidate compounds defined herein are also applicable as the therapeutic compounds that may be used in any of the personalized prognostic and therapeutic methods disclosed by the present disclosure.

It should be further appreciated that the screening method disclosed herein may be further applicable for identifying any compound that modulates any physiological and/or environmental condition or state of a subject or a media and/or habitat (and/or environmental habitat), provided that the sample is obtained from a subject and/or media and/or habitat displaying the specific physiological and/or environmental condition or state, and the phase variation value determined for a sample contacted with the candidate compound is compared to a predetermined a reference phase variation value determine for population of subjects displaying the specific condition or media characterized by the specific condition.

A further aspect of the present disclosure relates to a kit comprising:
(a) at least one detecting molecule for identifying at least one phase variation in at least one locus of at least one microorganism in at least one sample of a subject or media and/or habitat, to obtain at least one phase variation value of the sample. In some embodiments, the kit optionally further comprises at least one of: (b) pre-determined calibration curve/s or predetermined reference phase variation value/s pre-determined for at least one physiological or environmental condition or state in a subject or a media and/or habitat; and/or (c), at least one control sample.

In some embodiments, the disclosed kits may be adapted for performing any of the method according to the present disclosure. It should be appreciated that the components in the kit may depend on the method of detection and are not limited to any method. Some embodiments of the present disclosure concern a kit that further comprises nucleic acid detecting molecule or a reagent. This detecting molecule can be used for conducting various sequencing methods, such as next generation sequencing (NGS), Sanger, nanopore sequencing and targeted sequencing for example, nucleic acid amplification-based assay, a Real-Time PCR, micro arrays, PCR, in situ Hybridization, Comparative Genomic Hybridization, and molecular weight-based assays such as Southern and/or Northern blotting techniques.

In some embodiments, the kit of the present disclosure further comprising at least one reagent for conducting an immunological assay selected from for example from flow cytometry (FACS), ELISA, radioimmunoassay (RIA), slot blot, dot blot, western blot, immunohistochemical assay, immunofluorescent assay and a radio-imaging assay. Accordingly, such kit may comprise antibodies, labeling material, in some embodiments reagents substrates and enzymes required to perform colorimetric or electrochemical reaction, optionally, secondary antibodies, filters, beads and any required solid support. In some embodiments, the kit of the present disclosure may further comprise at least one reagent for conducting a mass spectrometry assay. Such reagents may include trypsin, buffers, filters and the like, for peptide purification. In further embodiments, the kit of disclosed herein may further comprise at least one device, means or any reagent for obtaining a biological sample, from a subject, for example any cell, tissue or body fluid sample (needles, aspirators and the like).

In yet some further aspects thereof, the present disclosure provides a computer implemented method of for determining a physiological and/or environmental condition or state of a subject or a media and/or habitat. Specifically, the computer implemented methods are applicable for identifying at least one locus displaying phase variation, that characterizes at least one physiological and/or environmental condition or state of a subject or a media and/or habitat. The various steps of the disclosed methods are detailed in Example 5. In yet some further embodiments, the disclosed method may further comprise a training a machine learning model. Still further, the present disclosure provides a computer program product comprising instructions which, when the program is executed by a computer, cause the computer to carry out a method for identifying at least one locus displaying phase variation, that characterizes at least one physiological and/or environmental condition or state of a subject or a media and/or habitat. A further aspect of the present disclosure provides a computer program product operable in a computer and comprising instructions stored on a non-transitory computer-readable medium for a method of for identifying at least one locus displaying phase variation, that characterizes at least one physiological and/or environmental condition or state of a subject or a media and/or habitat. The present disclosure further provides a computer implemented methods of training machine learning model for identifying at least one locus displaying phase variation, that characterizes at least one physiological and/or environmental condition or state of a subject or a media and/or habitat. Still further, the present disclosure provides a computer system comprising at least one computer circuitry configured to execute a method for identifying at least one locus displaying phase variation, that characterizes at least one physiological and/or environmental condition or state of a subject or a media and/or habitat.

All scientific and technical terms used herein have meanings commonly used in the art unless otherwise specified. The definitions provided herein are to facilitate understanding of certain terms used frequently herein and are not meant to limit the scope of the present disclosure.

The term “about” as used herein indicates values that may deviate up to between 1% to 10%, thus, as used herein the term “about” refers to +10%. The terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, and/or parts, but only if the additional ingredients and/or steps do not materially alter the basic and novel characteristics of the claimed composition or method. Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between. As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements. Various embodiments and aspects of the present invention as delineated hereinabove and as claimed in the claims section below find experimental support in the following examples. Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof. It must be noted that, as used in this specification and the appended claims, the singular forms “a”, “an” and “the” include plural referents unless the content clearly dictates otherwise.

### EXAMPLES

### Experimental Procedures

### Mice

All mouse work was in accordance with protocols approved by the local IACUC committee under approval numbers: IL-151-10-21 and IL-105-06-21. 4-5 weeks-old Germ-Free (GF) C57BL/6 mice (males and females) from the Technion colony were used. Mice were housed and maintained in a GF care facility and were provided with food and water ad libitum; they were exposed to a 12:12 h light-dark cycle at room temperature.

Humanized mice were generated by oral gavage of GF C57BL/6 mice with 200 μl mix of human fecal microbiota and cultured B. fragilis NCTC 9343. To prepare this mix, human feces from a single healthy human donor were suspended in sterile PBS 1:10 w/v (1 gram feces was suspended in 10 ml of sterile PBS). Next, 10⁸ CFUs of B. fragilis NCTC 9343 were resuspended in 1 ml of sterile PBS and added to 1 ml of fecal supernatant. B. fragilis NCTC 9343 were grown on Brain Heart Infusion agar plates (BHI, BD BBLTM) supplemented with 5 μg/ml hemin (Alfa Aesar) in 1 N NaOH and 2.5 g/ml vitamin K (Thermo Fisher Scientific) in 100% EtOH, at 37° C. in an anaerobic chamber, 85% N2, 10% CO2, 5% H2 (COY). The progeny of these mice were co-housed, randomized, and grouped before treatment.

Gnotobiotic mice monocolonized with B. fragilis were created by oral gavage of GF C57BL/6 mice with 200 μl of 5×10⁸ CFU\ml of B. fragilis NCTC 9343, grown in the same conditions as above. The mice were co-housed, randomized, and grouped before treatment. Both gavages, in humanized and monocolonized mice, were performed once.

For phage immunomodulation studies, GF mice were gavaged twice (on day 0 and on day 2) with B. fragilis NCTC 9343 (10⁸ CFU in 200 μl Bacteroides phage recovery medium (BPRM) media) and bacteriophage Barc2635 (10⁹ PFU in 200 μl 0.22-μm-filtered BPRM), B. fragilis only (10⁸ CFU in 200 μl BPRM media), or growth media as control. On day 10 mice were sacrificed for bacteria and immune phenotyping.

### DSS Model

For acute DSS-induced colitis, humanized mice were treated with 3% Dextran sodium sulfate in their drinking water for 9 days followed by 19 days recovery before sacrificing. The control animals were administered distilled water. Fecal samples were obtained on days 0, 6, 9, 14, 21, and 28, for further analyses.

### Inflammation Measurement

Calprotectin concentration in stool was determined as a marker of intestinal inflammation. Fecal supernatants were prepared after suspension in 1:10 sterile PBS and centrifugation for 15 minutes in 4500×g. Enzyme-linked immunosorbent assay (ELISA) was performed using Mouse S100A8/S100A9 Heterodimer kit according to the manufacturer protocol [R&D systems]. Mice weights were assessed using electronic scale at the same daytime.

qPCR and Primers

DNA was extracted from fecal samples using ZymoBIOMICS DNA Miniprep Kit [Zymo research]. The ‘ON/OFF’ status of the PSA gene in the extracted DNA samples was determined using a quantitative polymerase chain reaction (qPCR) using SYBR® Green mix. Two sets of primers were designed to target the PSA locus. One set was used as a proxy to the number of bacteria in the samples and targeted UpaY, the first gene immediately downstream to the promoter region. The second set of primers targeted the promoter region and would only produce a product when the orientation is ‘ON’. The ratio of ‘ON’/‘OFF’ PSA orientation in the samples was calculated using the ddCT method calculated against the PSA locked ‘ON’ results (100% ‘ON’ orientation). UpaY gene expression was determined by RT-qPCR as follows: RNA was extracted from fecal samples using zymoBIOMICS RNA miniprep kit [Zymo research]. Reverse transcription of RNA to cDNA was performed using the qScript cDNA Synthesis Kit [Quantabio]. upaY mRNA levels were determined by qPCR using SYBR® Green mix [Thermo Fisher Scientific] with primers against rpsL as a reference gene. The 2-ΔΔCT method was employed for the specificity fold change tests.

TABLE 1
qPCR primers

| | | SEQ |
|---|---|---|
| | | ID |
| Seq name | Sequence | NO. |
| B_Frag_upa Y_F | CGCTCGGACAAAGAAGGACC | 1 |
| B_Frag_upaY_R | ACTTCTACCCTACGACGACGA | 2 |
| B_Frag_PSA_M | GGTGTTCCAAAAGACGAACGT | 3 |
| B_Frag_PSA_F | TGTGTAAATGATAGGAGGCTAGGG | 4 |
| rpsL_F | CCGAACTCTGCAATGCGTAA | 5 |
| rpsL_R | CGCGAACCAGTACGATTGAG | 6 |

### Sequencing

DNA samples underwent quality control by Qubit fluorescence analysis to determine concentration of DNA for downstream analysis (ThermoFisher, Cat. Q32850). Libraries were prepared using the Illumina Tagmentation DNA prep streamlined library preparation protocol according to manufacturer's instructions with a minimum of 50 ng of DNA starting mass and 8 cycles of PCR enrichment, ending with a fragment size of 550 bp. IDT for Illumina DNA/RNA UD indexes and Nextera DNA CD indexes were used (Illumina IDT, Cat. 20027213; Illumina Nextera, Cat. 20018708).

All libraries were diluted to 15 μM in 96-plex pools and validated on 100-cycle paired-ends read Miseq V2 runs (Illumina, Cat. MS-102-2002), before shipping to the US at 4 nM for sequencing on the Novaseq 6000 in S4 mode at 96-plex in a 300-cycle paired-end reads run, with an estimated read depth of 30 Gbp per sample (Illumina, Cat. 20028312). Final loading concentration of 600 pM. All sequencing runs were performed with a spike-in of 1% PhiX control library V3 (Illumina, Cat. FC-110-3001).

### Taxonomic Profiling

Raw reads were mapped to the NCBI nucleotide database using Centrifuge [Dhariwal A, et al. Nucleic Acids Res. 45 (W1):W180-W188. 2017]. Taxonomic annotations for each read were obtained using ‘least common ancestor’ algorithm, and then summarized across all reads to create counts per taxon. Raw counts were normalized using total sum scaling. Raw counts were normalized to percentages for relative abundance. Taxonomic data was filtered for taxa with a minimum of 0.01% relative abundance and detection in at least 20% of the samples.

Community profiling was performed using metaphlan4 v4.0.0 [Blanco-Míguez A, et al. Nature Biotechnology 2023. Published online Feb. 23, 2023], with mpa database vJan21. For each sample, the forward reads were first aligned against the mpa database using bowtie2 v2.3.5.1 [Langmead B, Salzberg S L. Nat Methods. 2012; 9 (4): 357] (flags --sam-no-hd --sam-no-sq --no-unal --very-sensitive). Next, the resulting sam file was analyzed by metaphlan4 with default parameters. In each analysis, species at abundance<=0.1% were ignored.

### Microbiome Analysis

Statistical analysis of Sequenced data was initially performed using MicrobiomeAnalyst [Dhariwal et al., Nucleic Acids Res. 2017] followed by comprehensive analysis using R packages: Phyloseq [McMurdie & Holmes, PLOS One 2013], Vegan [Simpson, 2009] and DESeq2 [Love et al., Genome Biol. 2014]. Differences in microbial taxa and functional modules were assessed by differential abundance analyses using DESeq2 [Love et al., Genome Biol. 2014]. Alpha diversity indices (Chaol and Shannon) were compared using the Kruskal-Wallis rank sum test. Beta diversity distance matrices (Bray-Curtis) were compared using the vegan package's function ADONIS, a multivariate ANOVA based on dissimilarity tests and visualized using PCoA (taxonomy) and PCA (functional). Results were visualized using the ggplot2 R package [Wickham, 2016].

### Identification of Phase Variable Sites

Representative Bacteroides species were selected from an in-house database of human-associated microbial species. The database was constructed from 118K metagenomics assembled genomes (MAGs) recovered from human-associated metagenomics samples. Taxonomy assignment was done using gtdbtk v2.0.0 and GTDB release 207 with the classify_wf program and default parameters. Overall, 36 Bacteroides species were identified of which 35 had species-level GTDB taxonomy assignments. For each of these species, the representative genome from GTDB was used for the Phasefinder analysis. The investors have also included the genomes of two additional B. fragilis strains, Akkermansia muciniphila, Phocaeicola dorei, and Parabacteroides distasonis. Overall, 41 genomes were included.

Phasefinder [Jiang X, et al. Science 363 (6423): 181-187. 2019] (v1.0) was used to identify phase variable sites in the metagenomics samples. The default parameters of Phasefinder were used. The results were filtered by removing identified sites with <20 reads supporting either the forward or reverse orientations combined from the paired-end method, and mean Pe_ratio<1% across all samples. To compare phase variation patterns of CD and UC patients with those of healthy controls, the investors focused on phase variable sites that displayed a difference of over 10% between at least one of the groups. The Wilcoxon rank sum test was employed to conduct these comparisons, and the Benjamini-Hochberg method was utilized to correct for multiple comparisons, with a false discovery rate (FDR) set at less than 0.1. Each invertible region was manually curated to assess its coding regions, gene annotations, and their putative functions. Briefly, genomic regions were visualized online using the NCBI Graphical Sequence Viewer (version 3.47.0). Invertible regions with coding sequences (CDS) within them were annotated according to the CDS name(s). Invertible regions lacking CDS within them were searched for CDSs that start in proximity to the invertible DNA sites (<200~bp). CDSs in the region (four upstream and four downstream) were used to assess the functionality of the region. Regions containing or in proximity to rRNA and tRNA genes were filtered from the comparisons as well as invertible regions with no CDSs start in proximity to the inverted repeats.

MinION library preparation and sequencing: The specificity region of BF9343_1757-1760 was amplified by PCR from a population of bacteria grown in vitro and in vivo from fecal content as described above. The primers annealed outside the invertible region. The amplicons were purified by Wizard SV Gel and PCR Clean-Up System (Promega) and measured by nanodrop.

Primers: Type1RM_hsdS_F: GACAATCGAGATGAAGAACAAC, as denoted by SEQ ID NO: 7, and Type1RM_hsdS_R: CCATAGGCGTATGATTTCCTG, as denoted by SEQ ID NO: 8.

DNA quantity was measured again using Qubit fluorometry (Thermo Fisher Scientific, Waltham, MA, USA). Nanopore sequencing libraries were prepared from 200 fmol purified amplicons using Ligation Sequencing Kit 1D (SQK-LSK109) and PCR-free Native Barcoding Expansion Kit (EXP-NBD104) (Oxford Nanopore Technologies, Oxford, England). The barcoded libraries were loaded and sequenced on the MinION device controlled by MinKNOW software (v.19.12.5) using MinION flow cells (FLO-MIN106D R9.4.1, Oxford Nanopore Technologies, Oxford, England) after quality control runs. The raw data were base called and demultiplexed by Guppy Basecalling Software (v. 3.3.3+fa743a6).

### MinION Data Analysis

Adapters and barcodes sequences were removed from the reads using Porechop (v0.2.4, available from https://github.com/rrwick/Porechop). Reads were oriented using the ‘Preparing Reads for Stranded Mapping’ protocol (Eccles, D. A. (2019). Protocols.io, Vol. 2019, pp. Protocol). The reads were aligned to the PCR forward primer using LASTAL (v.1060), and then reverse-complemented the reverse-oriented reads. The reads were combined to an all forward oriented file and cropped to the first 1300 bases using Trimmomatic (v.0.39). The reads were then split according to their alignment to the 1757-57 or 1757-60 5″ half sequences using LASTAL. Reads were mapped to the full sequences with Minimap2 (v.2.17-r941) using the -for-only and asm20 options. Mapped read counts were extracted from the Minimap2 SAM output using SAMtools (v.1.7).

MinION sequence data has been deposited in the NCBI sequence read archive (SRA) under the BioProject accession number: PRJNA948162.

### Patients, Recruitment, Ethics, Sample Collection

Recruitment of IBD patients for this study was conducted at the Rambam Health Care Campus (RHCC). The study was approved by the local institutional review boards with study numbers 0052-17 and 0075-09 in which all patients consented to be included in it. During therapy, patients were treated either with Infliximab or Humira for at least two weeks.

Fecal samples were collected by patients at home prior to their clinic visit and collected from each at the hospital. The samples were then stored at −80° C. until they were shipped to the laboratory for analysis.

Calprotectin levels were measured in each fecal sample using LIAISON Calprotectin (catalogue No. 318960) according to the manufacturer's instructions. The levels of calprotectin in the fecal samples were used as a measure of disease activity in IBD patients.

### In Vitro Assays

Bacteroides fragilis NCTC 9343 was grown in Brain Heart Infusion medium with supplements (BHIS) to OD₆₀₀~0.6 and centrifuged for 5 minutes in 4500×g. Then, bacterial pellets were washed twice with sterile PBS to discard of remaining BHIS components and then suspended with 1 mL M9 minimal media.

Patient fecal samples were suspended in 1:5 sterile PBS, centrifuged for 15 minutes in 4500×g. Supernatants were collected and filtered using the Medical Millex-VV Syringe Filter Unit, 0.22 μm, PVDF membrane. For the in vitro assay, B. fragilis was cultured in patient's fecal filtered supernatants and M9 minimal media in 1:25:25 ratio, respectively. 200 ul of each culture was collected for DNA extraction at OD₆₀₀~0.6.

### Gut Lamina Propria Preparation

For lamina propria immunophenotyping, mice colons were removed by cutting the colon from the cecum-colon junction to the anus. Fat tissue was carefully removed from colon tissue and further proceeded for single cell suspension preparation using lamina propria dissociation kit (Miltenyi), according to the manufacturer's protocol.

### Flow Cytometry

Cell preparations for flow cytometry analysis were done in 5 ml tubes or U shape 96 wells plates. Single cells were washed with PBS and stained for live/dead staining using 1:1000 in PBS, Zombie fixable viability dye (Biolegend) for 10 minutes, at room temperature, and washed once with FACS buffer, by centrifuge at 300×g for 5 minutes. For Fc receptor (FcR) blocking, cells were incubated with 0.5 μg CD16/CD32 antibody for 10 minutes on ice and proceeded to further staining without a washing step. Extracellular markers were stained with the relevant antibody panels for 30 minutes on ice and washed twice with FACS buffer, by centrifuge at 300×g for 5 minutes. After the last wash, cells were fixed with Foxp3 Fixation/Permeabilization working solution (Thermo) for 16 hours at 4° C. in the dark. For Intracellular staining, cells were permeabilized using 1× Foxp3 permeabilization buffer (Thermo) according to the manufacturer protocol. For intracellular blocking, 2 μl of 2% rat serum (Stemcell technologies) was added to each well for 15 minutes at room temperature and proceeded to further staining without a washing step.

### Example 1

### Multiple Bacterial Phase Variation Sites are Altered in Association with IBD

To understand whether phase variation occurs in IBD patients, the inventors identified and quantified genomic phase variation sites in publicly available databases (Table 2), using the Phasefinder software. The inventors analyzed 41 genomes representing different fecal human associated bacterial species, including mostly Bacteroides species. This analysis revealed phase variations in 188 sites, in 28 of the analyzed genomes, spanning from regulatory genes to outersurface molecules, including both invertible promoters as well as intergenic phase variable regions (FIG. 1B). FIG. 1A presents bacteria with F orientation (‘ON’ state) vs. R orientation (‘Off’ state) in different invertible phase variable regions in different bacteria which is compared between Healthy, CD (Crohn's Disease) and UC (Ulcerative Colitis) patients. The invertible regions whose orientations differed most significantly between IBD patients and controls were within or in proximity to SusC/SusD-like outer membrane transport systems and capsular polysaccharides (PS) promoters (FIG. 1B). In Bacteroides thetatiotaomicron, 4 out of 5 phase variable capsular polysaccharides inverted between the groups and in B. fragilis, 3 out of 7, where the anti-inflammatory polysaccharide A (PSA) was the most significant one. The PSA promoter showed a higher percentage of reverse oriented reads (compared to its reference genome sequence) in IBD patients (FIG. 1C). FIG. 1D shows inversions in CPS-3 promoter region. In the reference genome of B. fragilis NCTC9343 the promoter of PSA is in its ‘ON’ orientation, hence, the reverse orientation, found in IBD patients, represents the PSA promoter's ‘OFF’ orientation (FIG. 1C). The inventors further identified phase variations in PULs and polysaccharides promoters of Phocaeicola dorei, a bacteria shown to be correlated with disease activity in UC (FIG. 1E).

TABLE 2
The databases used for analysis of phase variation in IBD patients

| | | | | Non- | | |
|---|---|---|---|---|---|---|
| Cohort | N* | CD | UC | IBD | Country | Notes |
| IBDMDB | 1238 | 574 | 349 | 360 | United States | Longitudinal |
| HMP | 320 | | | 320 | United States | |
| (3 phases) | | | | | | |
| MetaHit | 122 | 4 | 21 | 97 | Denmark and | |
| | | | | | Spain | |
| 1000IBD | 331 | 205 | 126 | | the Netherlands | |
| GeversD_{—} | 50 | 36 | | 14 | United States | |
| 2014 | | | | | and Canada | |
| LewisJD_{—} | 303 | 303 | | | United States | Children with |
| 2015 | | | | | and Canada | Crohn's |
| | | | | | | Disease |
| | | | | | | Longitudinal, |
| | | | | | | treated |
| SUM | 2364 | 1122 | 496 | 791 | | |

*Number of samples included in the analysis.

### Example 2

### Gut Bacterial Genomic Phase Variation is Induced by Inflammation in a Dynamic and Reversible Manner

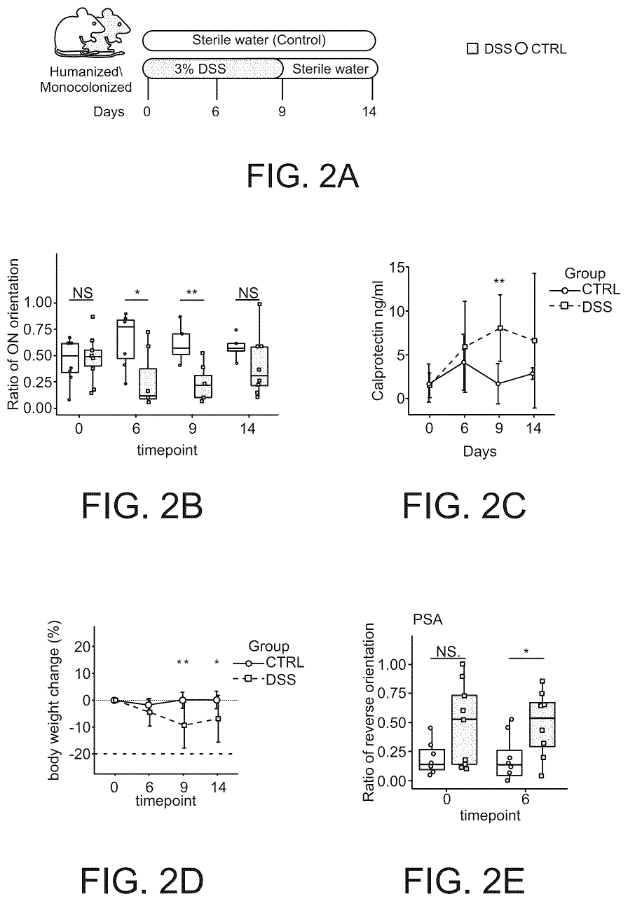

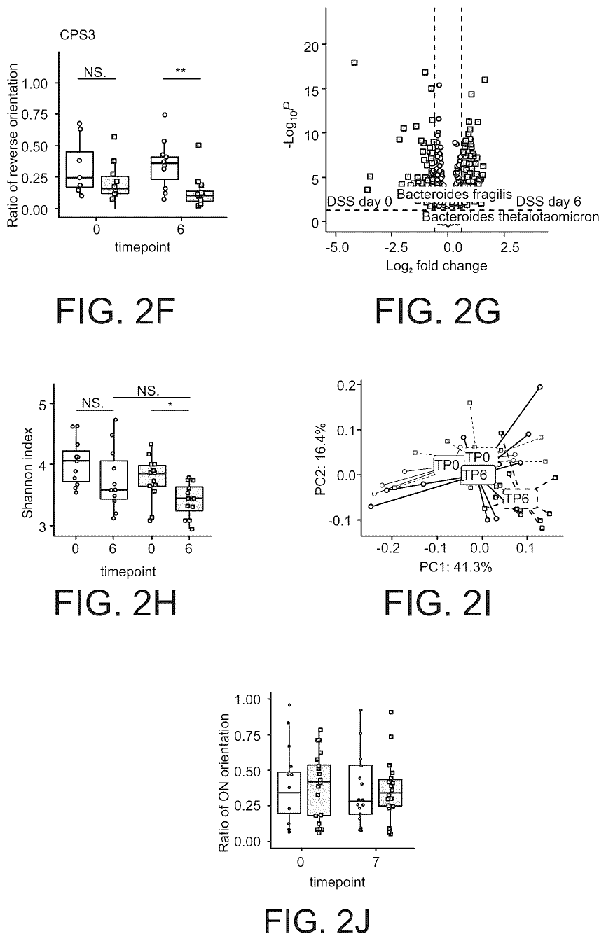

To assess the dynamics of the PSA promoter of B. fragilis under inflammatory conditions, the inventors designed a longitudinal experimental mouse model during which the PSA promoter orientation was analyzed over time-before, during and after the induced inflammation. To this end, a mouse model was designed, as illustrated by FIG. 2A: GF mice colonized with a healthy human microbiota, which included B. fragilis NCTC9343, termed “Humanized” mice. Experimental colitis was induced by adding 3% Dextran Sodium Sulfate (DSS) to the drinking water [Chassaing et al., 2014] (FIG. 2A) for 9 days after which DSS was replaced with water until the end of the experiment. During the course of the experiment the PSA promoter orientation was analyzed from stool, in multiple time-points, using quantitative PCR (qPCR), with primers targeted to the phase-variable promoter region. The PSA promoter orientation varied in a reversible manner, in the humanized mice, in concordance with the induced inflammation (FIG. 2B). At the beginning of the experiment, the promoter orientation aligned with that of the B. fragilis NCTC9343 reference genome, showing the ‘ON’ orientation in about 40% of the population (FIG. 2B). Six days after DSS was introduced, these percentages declined to 15%, implying that the promoter inverted to its ‘OFF’ orientation. The opposite state continued until day 9, after which it returned to the ‘ON’ orientation, similar to the control group, which did not receive DSS. To note, the PSA phase-variations were coherent with the measured Calprotectin levels in the stool (FIG. 2C, a commonly used biomarker for inflammation) and with mice weight loss (FIG. 2D). A metagenomics analysis of these mice was further performed to study alterations in bacterial composition and to apply the Phasefinder algorithm for extensive phase variation analysis. Phasefinder analysis on the “humanized” mice metagenomics data revealed phase variation of B. fragilis promoter of PSA, and in B. thetaiotaomicron promoter of CPS3, overlapping with the results of the human databases analysis (FIG. 2E, FIG. 2F).

On the composition level, the inventors identified alterations in the taxonomy of the bacteria appearing only in the DSS treated mice, whereas the control mice were stable throughout. Bacterial richness (alpha-diversity, Shannon Index) declined at day 6 under inflammatory conditions (FIG. 2H). Beta-diversity (Bray-Curtis distance) was altered upon DSS treatment, and each mouse reached a different bacterial composition after the inflammation was resolved (FIG. 2I). Inflamed mice showed a decrease in Aneurinibacillus, Prevotella, and Lactobacillus genera bacterial abundances, while showing an increase in species from the Enterobacteriaceae and Enterococcaceae families (FIG. 2G). The relative abundances of B. fragilis and B. thetaiotaomicron increased in inflamed mice, although to a lesser extent (FIG. 2G).

To assess the role of the host's inflammation on bacterial phase variation the experiment was repeated using Germ-Free (GF) mice, mono-colonized with B. fragilis NCTC9343. The PSA promoter orientation remained stable in the monocolonized mice over the course of the experiment, suggesting a role of the microbiota in the relative ‘OFF’ orientation of this promoter during inflammation (FIG. 2J).

### Example 3

### Investigating Viral Association to Genomic Orientation of the Polysaccharide a Promoter of B. fragilis

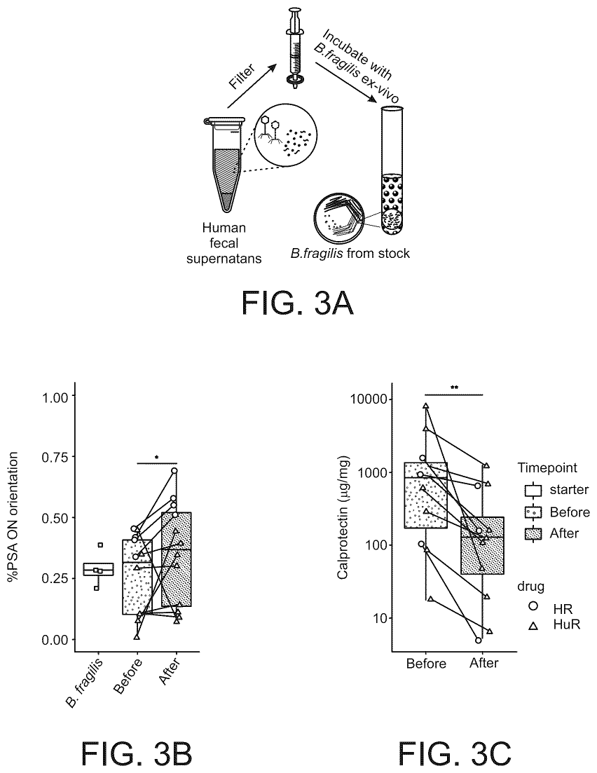

The inventors next sought to examine whether the gut inflamed environment can induce bacterial genomic phase variations of the polysaccharide A promoter of B. fragilis. To do so, B. fragilis was exposed to fecal filtrates from IBD patients. CD Patients were recruited from the Rambam Health Care Campus (RHCC) and fecal samples were collected before and after the patients were treated using infliximab (IFX). IFX is a common therapy for IBD, it is a chimeric monoclonal antibody against tumor necrosis factor-α (TNF-α), an inflammatory cytokine, which is increased in IBD patients. B. fragilis NCTC9343 was cultured in fecal filtrates until reaching mid log phase, and subsequently DNA was extracted for qPCR analysis of the PSA promoter orientation, as illustrated in the experimental scheme of FIG. 3A. This analysis revealed that B. fragilis exposed to fecal filtrates of patients before IFX treatment showed higher ratios of the PSA promoter ‘OFF’ orientation, whilst B. fragilis exposed to fecal filtrates after IFX treatment showed higher ratios of the PSA promoter ‘ON’ orientation, (FIG. 3B). This observation was in line with the fecal Calprotectin concentrations, which were reverse correlated with the PSA promoter orientation (FIG. 3C). These results demonstrated that PSA promoter genomic inversions are mediated by changes in the inflamed gut, switching towards the ‘ON’ orientation following reduction in inflammation (FIG. 3B). The fecal filtrates contain a mixture of bacterial and host metabolites, cytokines, antibodies, viruses, and bacteriophages.

### Example 4

### Phase Variation Loci in Melanoma Patients

The inventors analyzed metagenomic data of melanoma patients before starting immunotherapy comprising immune checkpoint blockade (ICB), specifically, an anti-Programmed death-ligand 1 (PDL1) antibody, from 9 different cohorts (Table 3). Previous studies [Jiang X, et al. Science 363 (6423): 181-187. 2019; Bian X, et al. Front Microbiol. 10:2259. 2019; Zhang T, et al. Appl Microbiol Biotechnol. 104 (23): 10203-10215. 2020] tested the correlation between the gut microbiome composition at baseline to immunotherapy response. However, non-conclusive results and differences between databases were observed. As a different analysis view, the inventors used the Phasefinder software for phase variation identification in reference genome of 41 commensal bacteria. This analysis uncovers the potential functionality changes in the cohorts.

The analysis revealed that responders had a higher ‘OFF’ orientation in three phase variation loci in the Bacteroides stercorirosoris strain DSM 26884, identified at high probability as the (1) promotor that chooses the expressed gene helix-turn-helix transcriptional regulator or DUF6198 family protein (NZ_FQZN01000001.1:394373.394411.394459.394497: IR1 &IR2 are between 2 genes that start from there BUC01_RS01270 & BUC01_RS01275. This section has 100% fit in Bacteroides cellulosilyticus strain WH2), (2) promotor that chooses the expressed gene HAD family phosphatase or MATE family efflux transporter (NZ_FQZN01000052.1:8795.8833.8881.8919: IR1 &IR2 are between 2 genes that start from there BUC01_RS23265 & BUC01_RS23270. In Bacteroides cellulosilyticus, it is equivalent to NZ_FQZN01000001.1:394373.394411.394459.394497), and (3) promotor of the gene ATP-binding protein (NZ_FQZN01000013.1:53467.53505.53553.53591: R1 &IR2 are between 2 genes that one start there BUC01_RS11220. this is the same sequence (the phase sequence) of Bacteroides cellulosilyticus as NZ_FQZN01000001.1:394373.394411.394459.394497). All these phase variation loci fitted loci within Bacteroides cellulosilyticus strain WH2 genome.

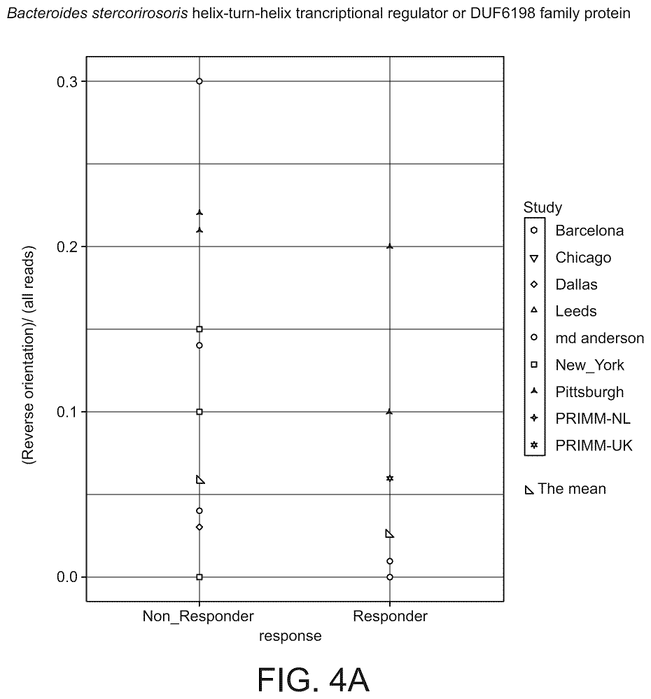

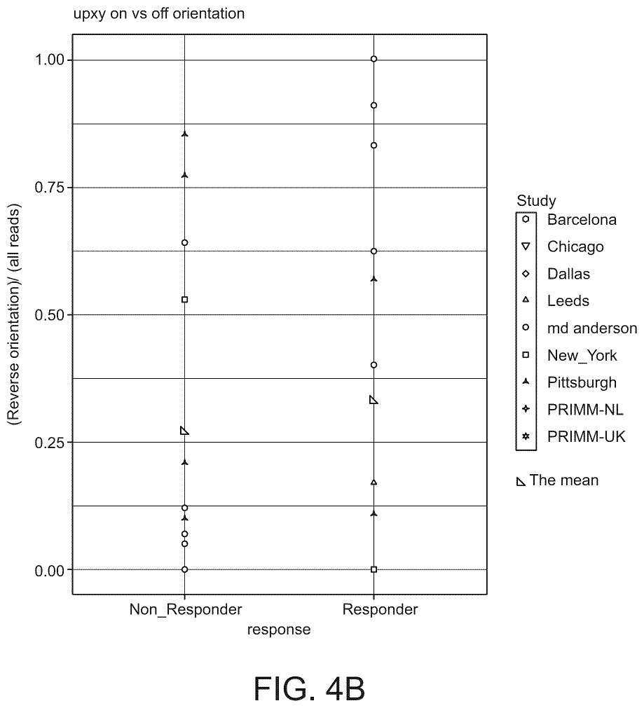

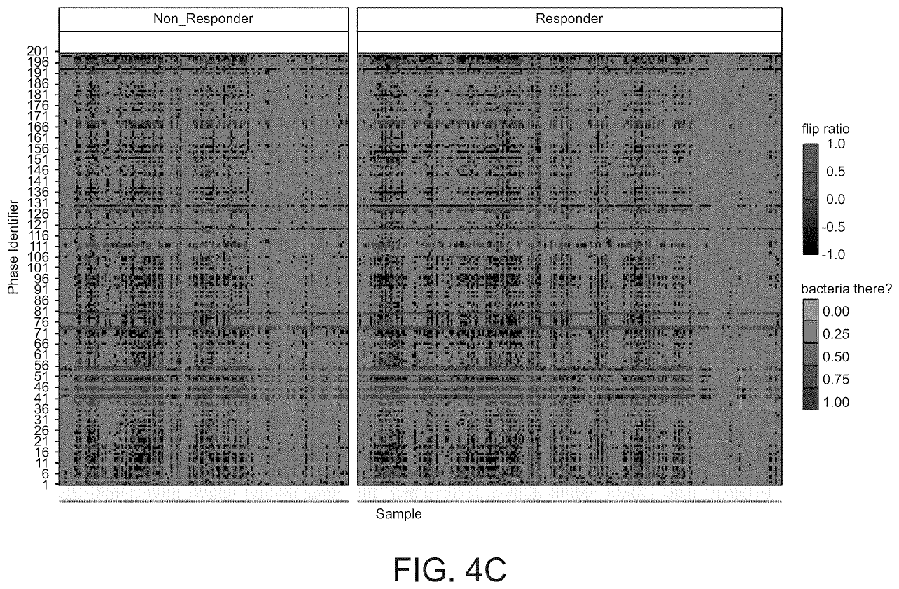

FIG. 4A demonstrate the ratio of reversed orientation in samples from responders vs. samples from non-responders in the different cohorts. There is a mild yet significant difference in the mean percentage of reversed orientation (triangle) of the tested region. This region has the same sequence as 8 different regions throughout the genome of B. cellulosilyticus indicating that these bacteria might have importance as well.

The inventors also used the data from 2021 Wargo science [Jiang X, et al. Science. 363 (6423): 181-187. 2019] to identify phase variation in the same reference genome of 41 commensal bacteria using the Phasefinder software. The 2021 Wargo science database analysis confirmed the analysis of the 9-cohort database, demonstrating that responders had a higher ‘OFF’ orientation in the same three phase variation loci within the genome of the Bacteroides stercorirosoris strain DSM 26884. In addition, a significant difference between responders and non-responders (without multiple hypothesis correction) was observed in a locus within the genome of Bacteroides ovatus strain ATCC 8483, identified with high probability as the promotor of the gene (or complex of genes) UpxY family transcription antiterminator (NZ_CP012938.1:1113559.1113590.1113835.1113866: R1 &IR2 are between 2 genes that one start there Bovatus_RS04390). In contrast, responders had no higher ‘OFF’ orientation in responders in a phase variation loci within the genome of Phocaeicola dorei isolate MGYG-HGUT-02478, identified at high probability as the gene TonB-dependent receptor (NZ_LR699004.1:641745.641762.642002.642019: chromosome 1, IR1 comes before gene FYB91_RS02215, IR2 before that closer to a gene that ends there). FIG. 4B demonstrate the ratio of ON/OFF orientation in the Bacteroides ovatus upxy promotor region. The md Anderson samples have difference between responders and non-responders.

The inventor then focused on Bacteroides ovatus. In different strains of B. ovatus, a phase variation was observed in the promoter of the UpxY regulatory gene but with slightly different phase variable regions.

FIG. 4C demonstrates the orientation ratio between ‘OFF’ and ‘ON’ in different phase variation regions identified within the genome of B. ovatus comparing responders to non-responder patients. In some of the regions, a higher level of reversed orientation (‘OFF’) was observed as compared to non-responders, for example in row 60 (FIG. 4C).

TABLE 3
Database used for the analysis of phase variation in melanoma patients

| | Sampling | | |
|---|---|---|---|
| Cohort | location | Patients | Reference |
| 1 | Houston, TX | 174 | [Jiang X, Science. 363(6423):181-187. 2019] |
| 2 | Netherlands | 55 | [Bian X, et al. Front Microbiol. 10:2259. 2019] |
| 3 | UK | 55 | [Bian X, et al. Front Microbiol. 10:2259. 2019] |
| 4 | Leeds | 18 | [Bian X, et al. Front Microbiol. 10:2259. 2019] |
| 5 | Barcelona | 12 | [Bian X, et al. Front Microbiol. 10:2259. 2019] |
| 6 | Pittsburgh | 63 | [Zhang T, al. Appl Microbiol Biotechnol. |
| | | | 104(23):10203-10215. 2020] |
| 7 | NYC | 27 | [Zhang T, al. Appl Microbiol Biotechnol. |
| | | | 104(23):10203-10215. 2020] |
| 8 | Chicago | 40 | [Zhang T, al. Appl Microbiol Biotechnol. |
| | | | 104(23):10203-10215. 2020] |
| 9 | Houston, TX | 25 | [Zhang T, al. Appl Microbiol Biotechnol. |
| | | | 104(23):10203-10215. 2020] |

### Example 5

### Developing the Identiphase Algorithm

The inventors next developed an algorithm that identifies bacterial genomic alterations in DNA, based on DNA sequencing data and quantifies the ratios of the genome varied bacteria. The algorithm is used to identify bacterial phase variable that is used as biomarkers associated with disease conditions and response to treatment.

### Algorithm

1. Prepare a bowtie2-database from the genome.
2. Create forward and reverse files with the first 50 base pairs (bp) of the reads.
3. Map the 50 bp-reads to the reference genome using bowtie2.
4. For inversions:
a. Look for PE (paired-end) reads that align to the genome in the same orientation.
b. Identify “hotspot regions”: these are windows of a certain size in which many same orientation reads fall. As initial attempt, the inventors begin with a sliding window of size insert-size*2.
c. For each hotspot region:
   i. Find its inversion location. This is done by looking for reads that are partially aligned or looking at reads that are unmapped and then we will try to blast them against the hotspot region. The inventors considered inverted but it may fail to identify some of the inverted repeats, and it does not use specific information that the inventors have.
   ii. Look for the inverted repeat in the paired hotspot regions.
   iii. Count the number of proper orientation PE-reads that are mapped to it.
   iv. If the number is >0 then this is a phase variation instance.
5. For insertions:
a. Look for PE reads that are mapped in the correct orientation but with larger than expected insert-size. A sliding window is applied with a threshold as before.
b. If the region contains no PE reads with an OK insert-size, reject the region.
c. Count the number of PE-reads with OK insert-size.
d. If the number is >0 then this is a phase variation instance.
   Identify regions in proximity to promoter sequences or located within an open reading frame (ORF).

1.-50. (canceled)

51. A method for modulating a physiological and/or environmental state and/or condition in a subject in need thereof, and/or a media and/or habitat, the method comprising:
(I) determining a physiological and/or environmental condition or state of a subject or a media and/or habitat, by the steps of:
(a) determining phase variation in at least one locus of at least one microorganism in at least one sample of said subject or media and/or habitat, to obtain at least one phase variation value of said sample; and
(b) classifying the subject and/or media and/or habitat as displaying said physiological and/or environmental condition or state, if the at least one phase variation value obtained for said sample in step (a), is positive with respect to a reference phase variation value pre-determined for said physiological and/or environmental condition or state, or with respect to a phase variation value determined for at least one control sample, thereby determining the physiological or environmental state for a subject or a media and/or habitat; and
(II) subjecting said subject and/or media and/or habitat classified as displaying said physiological and/or environmental condition or state to at least one physiological condition and/or compound that modulates said physiological condition and/or state, thereby modulating said physiological and/or environmental state and/or condition.

52. The method according to claim 51, wherein determining a physiological and/or environmental condition or state of a subject or a media and/or habitat in step (I), further comprising the step of identifying at least one locus displaying phase variation characterizing at least one physiological and/or environmental condition or state of a subject or a media and/or habitat, by the steps of:
(i) analyzing at least one phase variable region in the genome of at least one microorganism in at least one sample of at least one subject or media and/or habitat displaying said at least one physiological and/or environmental condition or state, and in at one sample of at least one subject or media and/or habitat that do no display said at least one physiological and/or environmental condition or state; and
(ii) mapping structural difference/s detected between the at least one phase variable region/s in the genome of at least one microorganism in the at least one sample of at least one subject or media and/or habitat displaying said at least one physiological and/or environmental condition or state, and the genome of at least one microorganism in the at least one sample of at least one subject or media and/or habitat that do not display said at least one physiological and/or environmental condition or state; thereby identifying at least one locus displaying phase variation characterizing at least one physiological and/or environmental condition or state of a subject or a media and/or habitat, optionally, wherein the step of identifying at least one locus displaying phase variation characterizing at least one physiological and/or environmental condition or state of a subject or a media and/or habitat, is performed prior to determining a physiological and/or environmental condition or state of a subject or a media and/or habitat.

53. The method according to claim 51, wherein said physiological state and/or condition of a subject comprises pathological condition/s and/or health condition/s in said subject, said physiological state and/or condition at least one of: an immunological state and/or condition, a metabolic state, diet, behavioral/mental state and/or condition.

54. The method according to claim 53, wherein said immunological state comprises and/or reflects at least one immune-related disorder in said subject, optionally, wherein at least one of:
(a) wherein said immune-related disorder comprises at least one of inflammatory disorder, an infectious disease, a proliferative disorder, an autoimmune disorder, an immune-deficiency condition, a neurodegenerative and/or cognitive and/or mental disorder, a metabolic disorder, and a condition involving at least one wound in at least one tissue and/or organ of said subject;
(b) wherein said inflammatory condition is inflammatory bowel disease (IBD); and
(c) wherein said proliferative disorder is at least one malignant neoplastic disorder.

55. The method according to claim 51, wherein said microorganism is at least one microorganism residing within at least one microbiome community of said subject and/or media and/or habitat, optionally at least one of:
(a) wherein said microbiome is a gut microbiome;
(b) wherein said gut microbiome comprises at least one of: bacteria, archaea, fungi, algae, protists, viruses, and bacteriophages;
(c) wherein said bacteria comprise at least one bacterium of at least one phylum selected from Bacteroidota, Verrucomicrobiota, proteobacteria, actinobacteria, firmicutes and Tenericutes;
(d) wherein said Bacteroidota bacterium is of the genus Bacteroides, Bacteroidia, Bacteroidales, Bacteroidaceae and Phocaeicola; and
(e) wherein said bacteria comprises at least one of Bacteroides fragilis, Bacteroides thetaiotaomicron, Phocaeicola dorei, Bacteroides cellulosilyticus, Bacteroides ovatus, Bacteroides stercorirosoris, or any isolate or species thereof.

56. The method according to claim 51, wherein at least one of:
(a) said phase variation in at least one locus comprise phase variation/s in at least one intergenic region/s and/or intragenic region/s;
(b) wherein said at least one locus comprises nucleic acid sequence/s encoding and/or regulating at least one outersurface and/or internal molecule and/or at least one molecule that modify or regulate said at least one outersurface and/or internal molecule/s; optionally, said at least one locus comprises at least one of polysaccharide utilization loci (PUL), SusC/D, capsular polysaccharide (CPS), ribosomal RNA (rRNA) 23S, rRNA 16S, fimbria, transposase, HsdS, outer membrane protein A (OmpA), Tetracycline resistance protein (Tet(Q) and Transfer RNA (tRNA), helix-turn-helix transcriptional regulator or DUF6198 family protein, HAD family phosphatase or MATE family efflux transporter, ATP-binding protein, UpxY family transcription antiterminator, TonB-dependent receptor; and
(c) wherein said bacterial outersurface molecules comprise CPS, and said CPS comprise polysaccharides (PS), and wherein said PS comprise polysaccharide A (PSA).

57. The method according to claim 51, wherein at least one of:
(a) phase variation is determined in at least one of the PSA locus, the CPS locus and any regulatory sequences thereof; and
(b) wherein said phase variations comprise at least one inversion in at least one promoter region of at least one gene residing in said PSA and/or CPS loci, thereby converting the ON/OFF orientation of said at least one promoter region/s.

58. The method according to claim 51, wherein said phase variations comprise at least one inversion in at least one promoter region of at least one gene residing in said helix-turn-helix transcriptional regulator or DUF6198 family protein, HAD family phosphatase or MATE family efflux transporter, ATP-binding protein, UpxY family transcription antiterminator, TonB-dependent receptor loci, thereby converting the ON/OFF orientation of said at least one promoter region/s.

59. The method according to claim 51, wherein phase variation or phase variation products are determined using nucleic acid-based level and/or amino-acid-based detecting molecules; optional, at least one of:
(a) wherein determination of phase variation or phase variation products comprises at least one of sequencing, amplification, hybridization, and molecular-weight based assays; and
(b) wherein determination of phase variation or phase variation products comprises at least one of affinity assay/s, molecular weight-based assay/s, scattering and mass spectrometry assay/s (MS).

60. The method according to claim 51, wherein said sample is a biological and/or environmental sample, optionally, wherein said biological sample comprises at least one of a body fluid and/or secretions and/or tissue sample.

61. The method according to claim 51, wherein said subject is at least one organism of the biological kingdom Animalia or of the biological kingdom Plantae, optionally, wherein said subject of the biological kingdom Animalia is a mammalian subject.

62. The method according to claim 51, wherein the step of determining a physiological and/or environmental condition or state of a subject or a media and/or or habitat in (I), is for detecting a pathological condition in a subject, the method comprising:
(a) determining phase variation in at least one locus of at least one microorganism in at least one sample of said subject, to obtain at least one phase variation value of said sample for at least one of said loci in said at least one microorganism; and
(b) classifying the subject as affected by, and/or suffering from said pathologic disorder, if the at least one phase variation value obtained for said sample in step (a), is positive with respect to a reference phase variation value pre-determined for said pathologic disorder, or with respect to a phase variation value determined for at least one control sample, thereby detecting said pathological disorder in said subject.

63. A method for treating, preventing, inhibiting, reducing, eliminating, protecting or delaying the onset of at least one pathologic disorder in a subject, the method comprising the steps of:
(I) detecting a pathological condition in a subject by the steps of:
(a) determining phase variation in at least one locus of at least one microorganism in at least one sample of said subject, to obtain at least one phase variation value of said sample for at least one of said loci in said at least one microorganism; and
(b) classifying the subject as affected by, and/or suffering from said pathologic disorder, if the at least one phase variation value obtained for said sample in step (a), is positive with respect to a reference phase variation value pre-determined for said pathologic disorder, or with respect to a phase variation value determined for at least one control sample, thereby detecting said pathological disorder in said subject; and
(II) administering to a subject classified as affected by, and/or suffering from said disorder a therapeutically effective amount of at least one therapeutic compound.

64. The method according to claim 63, wherein step (II) is for detecting at least one immune-related disorder in a subject, the method comprising:
(a) determining phase variation in at least one of the PSA, CPS, helix-turn-helix transcriptional regulator or DUF6198 family protein, HAD family phosphatase or MATE family efflux transporter, ATP-binding protein, UpxY family transcription antiterminator, TonB-dependent receptor loci of at least one bacterium in at least one sample of said subject, to obtain at least one phase variation value of said sample for at least one of said loci in at least one bacterium;
and
(b) classifying the subject as affected by, and/or suffering from said pathologic disorder, if the at least one phase variation value obtained for said sample in step (a), is positive with respect to a reference phase variation value pre-determined for said immune-related disorder, or with respect to a phase variation value determined for at least one control sample, thereby detecting said immune-related disorder in said subject.

65. The method according to claim 63, wherein said immune-related disorder is an inflammatory disorder being IBD, the method comprising:
(a) determining phase variation in at least one of the PSA and the CPS loci of at least one Bacteroidota bacterium in at least one sample of said subject, to obtain at least one phase variation value of said sample;
and
(b) classifying the subject as affected by, and/or suffering from IBD, if the at least one phase variation value obtained for said sample in step (a), is positive with respect to a reference phase variation value pre-determined for IBD patients, or with respect to a phase variation value determined for at least one control sample, thereby detecting IBD in said subject.

66. The method according to claim 63, wherein said immune-related disorder is a proliferative disorder, being melanoma, the method comprising:
(a) determining phase variation in at least one of the helix-turn-helix transcriptional regulator or DUF6198 family protein, HAD family phosphatase or MATE family efflux transporter, ATP-binding protein, UpxY family transcription antiterminator, TonB-dependent receptor loci of at least one Bacteroidota bacterium in at least one sample of said subject, to obtain at least one phase variation value of said sample;
and
(b) classifying the subject as affected by, and/or suffering from melanoma, if the at least one phase variation value obtained for said sample in step (a), is positive with respect to a reference phase variation value pre-determined for melanoma patients, or with respect to a phase variation value determined for at least one control sample, thereby detecting melanoma in said subject.

67. A method for personalized treatment of a subject suffering from a pathologic disorder, the method comprising the steps of:
(I) assessing responsiveness of a subject suffering from a pathologic disorder to at least one therapeutic agent or a treatment regimen comprising said at least one therapeutic agent, by the steps of:
(a) determining phase variation in at least one locus of at least one microorganism in at least one sample of said subject, to obtain at least one phase variation value of said sample for at least one of said loci in said at least one microorganism; and
(b) classifying the subject as:
(i) a responder subject to said at least one therapeutic agent or treatment regimen, if the at least one phase variation value obtained in step (a) for at least one sample obtained after the initiation of said treatment regimen and/or a sample of said subject contacted with said therapeutic agent, is negative with respect to a reference phase variation value pre-determined for said pathologic disorder, or with respect to a phase variation value determined for at least one control sample: or
(ii) a non-responder subject to said at least one therapeutic agent or treatment regimen, if the at least one phase variation value obtained in step (a) for at least one sample obtained after the initiation of said treatment regimen and/or a sample of said subject contacted with said therapeutic agent, is positive with respect to a reference phase variation value pre-determined for said pathologic disorder, or with respect to a phase variation value determined for at least one control sample; and
(II) selecting a treatment regimen based on said responsiveness;
wherein selecting a treatment regimen based on said responsiveness comprises one of:
(i) administering said therapeutic agent to a subject classified as a responder, and/or maintaining said treatment regimen for a subject classified as a responder; or
(ii) ceasing a treatment regimen comprising at least one therapeutic agent for a subject displaying disease relapse and/or loss of responsiveness, non-responsiveness, poor-responsiveness and/or drug-resistance.

68. The method according to claim 67, wherein at last one of:
(a) said bacteria comprise at least one bacterium of at least one phylum selected from Bacteroidota, Verrucomicrobiota, proteobacteria, actinobacteria, firmicutes and Tenericutes, optionally, said bacteria comprises at least one of Bacteroides fragilis, Bacteroides thetaiotaomicron, Phocaeicola dorei, Bacteroides cellulosilyticus, Bacteroides ovatus, Bacteroides stercorirosoris, or any isolate or species thereof; and
(b) wherein said phase variation in at least one locus comprise phase variation/s in at least one intergenic region/s and/or intragenic region/s.

69. The method according to claim 67, wherein said pathologic disorder is at least one immune-related disorder, and wherein said phase variation is determined in at least one of the PSA, CPS, helix-turn-helix transcriptional regulator or DUF6198 family protein, HAD family phosphatase or MATE family efflux transporter, ATP-binding protein, UpxY family transcription antiterminator, TonB-dependent receptor loci of at least one Bacteroidota bacterium in at least one sample of said subject, optionally, wherein said immune-related disorder is an inflammatory disorder being IBD, and wherein said therapeutic agent comprises an agent directed against an inflammatory cytokine, the method comprising:
(a) determining phase variation in at least one of the PSA and the CPS loci of at least one Bacteroidota bacterium in at least one sample of said subject, to obtain at least one phase variation value of said sample; and
(b) classifying the subject as:
(i) a responder subject to said agent directed against an inflammatory cytokine, if the at least one phase variation value obtained in step (a) for at least one sample obtained after the initiation of said treatment regimen and/or a sample of said subject contacted with said agent, is negative with respect to a reference phase variation value (or cutoff value) pre-determined for said IBD, or with respect to a phase variation value determined for at least one control sample; or
(ii) a non-responder subject to said at least one agent directed against an inflammatory cytokine, if the at least one phase variation value obtained in step (a) for at least one sample obtained after the initiation of said treatment regimen and/or a sample of said subject contacted with said agent, is positive with respect to a reference phase variation value pre-determined for said IBD, or with respect to a phase variation value determined for at least one control sample.

70. The prognostic method according to claim 67, wherein said immune-related disorder is a proliferative disorder, being melanoma, and wherein said therapeutic agent comprises at least one immune checkpoint inhibitor compound, the method comprising:
(a) determining phase variation in at least one of the helix-turn-helix transcriptional regulator or DUF6198 family protein, HAD family phosphatase or MATE family efflux transporter, ATP-binding protein, UpxY family transcription antiterminator, TonB-dependent receptor loci of at least one Bacteroidota bacterium in at least one sample of said subject, to obtain at least one phase variation value of said sample; and
(b) classifying the subject as:
(i) a responder subject to said at least one immune checkpoint inhibitor compound, if the at least one phase variation value obtained in step (a) for at least one sample obtained after the initiation of said treatment regimen and/or a sample of said subject contacted with said agent, is negative with respect to a reference phase variation value pre-determined for said melanoma, or with respect to a phase variation value determined for at least one control sample; or
(ii) a non-responder subject to said at least one immune checkpoint inhibitor compound, if the at least one phase variation value obtained in step (a) for at least one sample obtained after the initiation of said treatment regimen and/or a sample of said subject contacted with said at least one immune checkpoint inhibitor compound, is positive with respect to a reference phase variation value (or cutoff value) pre-determined for said melanoma, or with respect to a phase variation value determined for at least one control sample, optionally, wherein phase variation or phase variation products are determined using nucleic acid-based and/or amino-acid-based detecting molecules.
